(19) United States
(12) Patent Application Publication
(10) Pub. No.: US 2014/0256740 A1
(43) Pub. Date: Sep. 11, 2014

(54) COMPOUNDS AND METHODS

(71) Applicant: Tempero Pharmaceuticals, Inc., Cambridge, MA (US)

(72) Inventors: Erkan Baloglu, Stoneham, MA (US); Shomir Ghosh, Cambridge, MA (US); Mercedes Lobera, Cambridge, MA (US); Darby R. Schmidt, Arlington, MA (US)

(73) Assignee: Tempero Pharmaceuticals, Inc., Cambridge, MA (US)

(21) Appl. No.: 14/235,489

(22) PCT Filed: Jul. 27, 2012
(86) PCT No.: PCT/US12/48588

§ 371 Date: Jan. 28, 2014

Related U.S. Application Data
(60) Provisional application No. 61/513,121, filed on Jul. 29, 2011.
(60) Provisional application No. 61/521,838, filed on Aug. 10, 2011.
(60) Provisional application No. 61/533,913, filed on Sep. 13, 2011.

(51) Int. Cl. C07D 413/12; C07D 271/06; C07D 263/32; C07D 275/02; C07D 261/08; C07D 277/30

(52) U.S. Cl. CPC C07D 413/12; C07D 261/08; C07D 277/30; C07D 263/32; C07D 275/02; C07D 271/06
USPC 514/254.04; 548/247; 514/378; 548/204; 514/365; 548/236; 514/374; 548/249; 544/367; 546/272.1; 514/340; 548/214; 514/372; 548/131; 514/364

(57) ABSTRACT

The present invention relates to novel retinoid-related orphan receptor gamma (RORγ) modulators and their use in the treatment of diseases mediated by RORγ.

The present invention relates to novel retinoid-related orphan receptor gamma (RORγ) modulators and their use in the treatment of diseases mediated by RORγ.

### BACKGROUND OF THE INVENTION

Retinoid-related orphan receptors (RORs) are transcription factors which belong to the steroid hormone nuclear receptor superfamily (Jetten & Joo (2006) Adv. Dev. Biol. 16:313-355). The ROR family consists of three members, ROR alpha (RORα), ROR beta (RORβ), and ROR gamma (RORγ), each encoded by a separate gene (RORA, RORB, and RORC, respectively). RORs contain four principal domains shared by the majority of nuclear receptors: an N-terminal A/B domain, a DNA-binding domain, a hinge domain, and a ligand binding domain. Each ROR gene generates several isoforms which differ only in their N-terminal A/B domain. Two isoforms of RORγ have been identified: RORγ1 and RORγt (also known as RORγ2). RORγ is a term used to describe both RORγ1 and/or RORγt.

While RORγ1 is expressed in a variety of tissues including thymus, muscle, kidney and liver, RORγt is exclusively expressed in the cells of the immune system. RORγt has been identified as a key regulator of Th17 cell differentiation. Th17 cells are a subset of T helper cells which produce IL-17 and other proinflammatory cytokines. Th17 cells have been shown to have key functions in several mouse autoimmune disease models including experimental autoimmune encephalomyelitis (EAE) and collagen-induced arthritis (CIA). In addition, Th17 cells or their products have been shown to be associated with the pathology of a variety of human inflammatory and autoimmune disorders including multiple sclerosis, rheumatoid arthritis, psoriasis, Crohn's disease and asthma (Jetten (2009) Nucl. Recept. Signal. 7:e003 Manel et al. (2008) Nat. Immunol. 9:641-649). The pathogenesis of chronic autoimmune diseases including multiple sclerosis and rheumatoid arthritis arises from the break in tolerance towards self-antigens and the development of auto-aggressive effector T cells infiltrating the target tissues. Studies have shown that Th17 cells are one of the important drivers of the inflammatory process in tissue-specific autoimmunity (Steinman (2008) J. Exp. Med. 205:1517-1522; Leung et al. (2010) Cell. Mol. Immunol. 7:182-189). There is evidence that Th17 cells are activated during the disease process and are responsible for recruiting other inflammatory cells types, especially neutrophils, to mediate pathology in the target tissues (Korn et al. (2009) Annu. Rev. Immunol. 27:485-517).

RORγt plays a critical role in the pathogenic responses of Th17 cells (Ivanov et al. (2006) Cell 126:1121-1133). RORγt deficient mice produce few Th17 cells. In addition, RORγt deficiency resulted in amelioration of EAE. Further support for the role of RORγt in the pathogenesis of autoimmune or inflammatory diseases can be found in the following references: Jetten & Joo (2006) Adv. Dev. Biol. 16:313-355; Meier et al. (2007) Immunity 26:643-654; Aloisi & Pujol-Borrell (2006) Nat. Rev. Immunol. 6:205-217; Jager et al. (2009) J. Immunol. 183:7169-7177; Serafini et al. (2004) Brain Pathol. 14:164-174; Magliozzi et al. (2007) Brain 130:1089-1104; Barnes (2008) Nat. Rev. Immunol. 8:183-192.

In light of the role RORγ plays in the pathogenesis of diseases, it is desirable to prepare compounds that modulate RORγ activity, which can be used in the treatment of diseases mediated by RORγ.

### SUMMARY OF THE INVENTION

The invention is directed to novel RORγ modulators and their use in the treatment of diseases mediated by RORγ. Specifically, the invention is directed to compounds according to Formula (I):

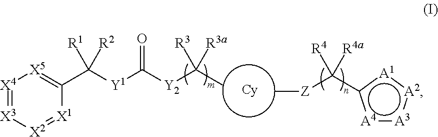

wherein:

m is 0, 1, or 2;

n is 0, 1, 2, or 3;

X¹, X², X³, X⁴, and X⁵ are each independently selected from N, N⁺—O⁻, CH, and CR⁵, wherein 0-3 of X¹, X², X³, X⁴, and X⁵ are N or N⁺—O⁻ and 1-3 of X¹, X², X³, X⁴, and X⁵ are CR⁵; provided that when zero of X¹, X², X⁴, and X⁵ are N or N⁺—O⁻ and X³ is CR⁵, 1-2 of X¹, X², X⁴, and X⁵ are CR⁵;

one of Y¹ and Y² is O or NR⁸ and the other is a bond;

or X¹ is CR⁵, Y¹ is NR⁸, Y² is a bond, and R⁵ and R⁸ taken together with the atoms to which they are attached form a five to seven membered ring, optionally containing an additional heteroatom selected from oxygen, nitrogen, and sulfur, which ring is optionally substituted by (C₁-C₄)alkyl;

Cy is (C₃-C₈)cycloalkyl, heterocycloalkyl, phenyl, or 5- or 6-membered heteroaryl, each of which is optionally substituted one, two, or three times, independently, by (C₁-C₆)alkyl, (C₁-C₆)haloalkyl, (C₃-C₆)cycloalkyl, halogen, oxo, cyano, hydroxyl, hydroxy(C₁-C₆)alkyl, (C₁-C₆)alkoxy, —((C₀-C₃)alkyl)NHCO₂R⁷, —((C₀-C₃)alkyl)N((C₁-C₄)alkyl)CO₂R⁷, —((C₀-C₃)alkyl)NHC(O)R⁷, —((C₀-C₃)alkyl)N((C₁-C₄)alkyl)C(O)R⁷, —((C₀-C₃)alkyl)CO₂R⁷, —((C₀-C₃)alkyl)CONR⁷R⁸, —((C₀-C₃)alkyl)C(O)R⁷, (C₁-C₄)alkoxy(C₁-C₆)alkyl, amino(C₁-C₆)alkyl, ((C₁-C₄)alkyl)((C₁-C₄)alkyl)amino(C₁-C₆)alkyl, (C₁-C₄)alkylamino(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, or heterocycloalkyl;

Z is O, S, SO₂, C═O, NR⁶, or a bond;

A¹, A², A³, and A⁴ are each independently selected from N, NR⁶, O, S, CH, and CR¹⁰, wherein one of A¹, A², A³, and A⁴ is NR⁶, O, or S, 0-2 of A¹, A², A³, and A⁴ are CR¹⁰, and 0-3 of A¹, A², A³, and A⁴ are CH or N;

R¹ is (C₃-C₆)alkyl, (C₃-C₆)haloalkyl, (C₃-C₈)cycloalkyl, (C₃-C₆)alkoxy, (C₁-C₆)alkoxy(C₁-C₂)alkyl, aryl, heteroaryl, aryl(C₁-C₆)alkyl, heteroaryl(C₁-C₆)alkyl, or heterocycloalkyl, each of which is optionally substituted one, two, or three times, independently, by R⁵;

R² is hydrogen, (C₁-C₆)alkyl, or (C₁-C₆)haloalkyl;

or R¹ and R² taken together with the carbon atom to which they are attached form a three to eight membered ring, optionally containing a heteroatom selected from oxygen, nitrogen, and sulfur, which ring is optionally substituted one, two, or three times, independently, by R⁵;

R³ and R^{3a} are each independently hydrogen, hydroxyl, (C₁-C₆)alkyl, (C₁-C₆)haloalkyl, halogen, (C₁-C₆)alkoxy, amino, (C₁-C₄)alkylamino, or ((C₁-C₄)alkyl)((C₁-C₄)alkyl)amino;

each R⁴ is independently selected from hydrogen, halogen, (C₁-C₆)alkyl, (C₁-C₆)haloalkyl, —CO₂R⁷, —CONR⁷R⁸, —OR⁹, and —NR⁸R⁹, wherein said (C₁-C₆)alkyl or (C₁-C₆)haloalkyl is optionally substituted by hydroxyl, —OR⁹, —CO₂R⁷, —CONR⁷R⁸, or —NR⁸R⁹;

each R^{4a} is independently selected from hydrogen, halogen, hydroxyl, amino, and (C₁-C₆)alkyl;

or R⁴ and R^{4a} taken together with the carbon atom to which they are attached form a three to eight membered ring, optionally containing a heteroatom selected from oxygen, nitrogen, and sulfur, which ring is optionally substituted by cyano, (C₁-C₄)alkyl, (C₁-C₄)haloalkyl, (C₃-C₆)cycloalkyl, —CO₂R⁷, —CONR⁷R⁸, hydroxyl, hydroxy(C₁-C₆)alkyl, (C₁-C₄)alkoxy, (C₁-C₄)alkoxy(C₁-C₆)alkyl, amino, (C₁-C₄)alkylamino, ((C₁-C₄)alkyl)((C₁-C₄)alkyl)amino, —NHCO₂R⁷, —N((C₁-C₄)alkyl)CO₂R⁷, —NHC(O)R⁷, or —N((C₁-C₄)alkyl)C(O)R⁷;

each R⁵ is independently selected from (C₁-C₆)alkyl, (C₁-C₆)haloalkyl, (C₃-C₆)cycloalkyl, halogen, cyano, hydroxyl, hydroxy(C₁-C₆)alkyl, (C₁-C₆)alkoxy, (C₁-C₄)alkoxy(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, and heterocycloalkyl;

R⁶ is hydrogen, (C₁-C₆)alkyl, (C₁-C₆)haloalkyl, (C₃-C₆)cycloalkyl, hydroxy(C₁-C₆)alkyl, (C₁-C₄)alkoxy(C₁-C₆)alkyl, —((C₀-C₃)alkyl)CO₂R⁷, —((C₀-C₃)alkyl)CONR⁷R⁸, aryl, heteroaryl, aryl(C₁-C₆)alkyl, heteroaryl(C₁-C₆)alkyl, or heterocycloalkyl;

R⁷ is hydrogen, (C₁-C₆)alkyl, (C₁-C₆)haloalkyl, (C₃-C₆)cycloalkyl, (C₁-C₄)alkoxy(C₁-C₆)alkyl, aryl, heteroaryl, aryl(C₁-C₆)alkyl, heteroaryl(C₁-C₆)alkyl, or heterocycloalkyl;

R⁸ is hydrogen, (C₁-C₆)alkyl, or (C₁-C₆)haloalkyl;

or R⁷ and R⁸ taken together with the nitrogen atom to which they are attached form a four to eight membered ring, optionally containing an additional heteroatom selected from oxygen, nitrogen, and sulfur, which ring is optionally substituted by (C₁-C₄)alkyl, (C₁-C₄)haloalkyl, (C₃-C₆)cycloalkyl, —CO₂H, —CO₂(C₁-C₄)alkyl, hydroxyl, hydroxy(C₁-C₆)alkyl, (C₁-C₄)alkoxy, (C₁-C₄)alkoxy(C₁-C₆)alkyl, amino, (C₁-C₄)alkylamino, or ((C₁-C₄)alkyl)((C₁-C₄)alkyl)amino;

R⁹ is —C(O)R⁷, —CO₂R⁷, —C(O)NR⁷R⁸, (C₁-C₆)alkyl, (C₁-C₆)haloalkyl, (C₃-C₆)cycloalkyl, aryl, heteroaryl, aryl(C₁-C₆)alkyl, heteroaryl(C₁-C₆)alkyl, or heterocycloalkyl, wherein said (C₁-C₆)alkyl, (C₁-C₆)haloalkyl, (C₃-C₆)cycloalkyl, aryl, heteroaryl, aryl(C₁-C₆)alkyl, heteroaryl(C₁-C₆)alkyl, or heterocycloalkyl is optionally substituted by —CO₂R⁷, —CONH₂, —CONH(C₁-C₄)alkyl, —CON((C₁-C₄)alkyl)((C₁-C₄)alkyl), hydroxyl, (C₁-C₄)alkoxy, amino, (C₁-C₄)alkylamino, ((C₁-C₄)alkyl)((C₁-C₄)alkyl)amino, —NHCO₂R⁷, —N((C₁-C₄)alkyl)CO₂R⁷, —NHC(O)R⁷, or —N((C₁-C₄)alkyl)C(O)R⁷;

or R⁸ and R⁹ taken together with the nitrogen atom to which they are attached form a four to eight membered ring, optionally containing an additional heteroatom selected from oxygen, nitrogen, and sulfur, which ring is optionally substituted by cyano, (C₁-C₄)alkyl, (C₁-C₄)haloalkyl, (C₃-C₆)cycloalkyl, —CO₂H, —CO₂(C₁-C₄)alkyl, —CONR⁷R⁸, hydroxyl, hydroxy(C₁-C₆)alkyl, (C₁-C₆)alkoxy, (C₁-C₄)alkoxy(C₁-C₆)alkyl, amino, (C₁-C₄)alkylamino, ((C₁-C₄)alkyl)((C₁-C₄)alkyl)amino, —NHCO₂R⁷, —N((C₁-C₄)alkyl)CO₂R⁷, —NHC(O)R⁷, or —N((C₁-C₄)alkyl)C(O)R⁷; and

R¹⁰ is (C₁-C₆)alkyl, (C₁-C₆)haloalkyl, (C₃-C₆)cycloalkyl, halogen, cyano, hydroxyl, hydroxy(C₁-C₆)alkyl, (C₁-C₆)alkoxy, (C₁-C₄)alkoxy(C₁-C₆)alkyl, —((C₀-C₃)alkyl)CO₂R⁷, —((C₀-C₃)alkyl)CONR⁷R⁸, amino(C₁-C₆)alkyl, ((C₁-C₄)alkyl)((C₁-C₄)alkyl)amino(C₁-C₆)alkyl, (C₁-C₄)alkylamino(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, or heterocycloalkyl;

or a salt thereof, particularly, a pharmaceutically acceptable salt thereof.

In one embodiment of this invention, the compound of Formula I does not include (2-(4-((3,5-dimethylisoxazol-4-yl)methoxy)phenyl)-N-((2-methoxyphenyl)(1-methyl-1H-imidazol-2-yl)methyl)acetamide).

In another aspect, this invention provides a pharmaceutical composition comprising a compound of Formula (I), or a pharmaceutically acceptable salt thereof, and a pharmaceutically acceptable excipient.

In another aspect, this invention provides for the use of a compound of Formula (I) or a pharmaceutically acceptable salt thereof for the treatment of diseases mediated by RORγ. The invention further provides for the use of a compound of Formula (I) or a pharmaceutically acceptable salt thereof as an active therapeutic substance in the treatment of a disease mediated by RORγ.

In another aspect, the invention provides a compound of Formula (I) or a pharmaceutically acceptable salt thereof for use in therapy.

In another aspect, the invention provides the use of a compound of Formula (I) or a pharmaceutically acceptable salt thereof in the manufacture of a medicament for the treatment of diseases mediated by RORγ.

Examples of such diseases for which compounds of Formula (I) may be used include autoimmune or inflammatory diseases such as multiple sclerosis, rheumatoid arthritis, psoriasis, uveitis, dry eye, glomerulonephritis, Crohn's disease and asthma, especially psoriasis

In yet another aspect, the invention is directed to methods of treating such diseases for example by administering to a patient (e.g. human) in need thereof an effective amount of a compound of Formula (I) or a pharmaceutically acceptable salt thereof.

### DETAILED DESCRIPTION OF THE INVENTION

As used herein, the term “alkyl” represents a saturated, straight, or branched hydrocarbon moiety. The term “(C₁-C₆)alkyl” refers to an alkyl moiety containing from 1 to 6 carbon atoms. Exemplary alkyls include, but are not limited to methyl, ethyl, n-propyl, isopropyl, n-butyl, isobutyl, s-butyl, t-butyl, pentyl, and hexyl. C₀alkyl means that no alkyl group is present in the moiety. Thus, —((C₀)alkyl)CONH₂ is equivalent to —CONH₂.

When the term “alkyl” is used in combination with other substituent groups, such as “haloalkyl”, “hydroxyalkyl”, “alkoxyalkyl”, “arylalkyl”, or “heteroarylalkyl”, the term “alkyl” is intended to encompass a divalent straight or branched-chain hydrocarbon radical. For example, “arylalkyl” is intended to mean the radical -alkylaryl, wherein the alkyl moiety thereof is a divalent straight or branched-chain carbon radical and the aryl moiety thereof is as defined herein, and is represented by, for example, the bonding arrangement present in a benzyl group (—CH₂-phenyl); “halo(C₁-C₄)alkyl” is intended to mean a radical having one or more halogen atoms, which may be the same or different, at one or more carbon atoms of an alkyl moiety containing from 1 to 4 carbon atoms, which is a straight or branched-chain carbon radical, and is represented by, for example, a trifluoromethyl group (—CF₃).

As used herein, the term “cycloalkyl” refers to a non-aromatic, saturated, cyclic hydrocarbon ring. The term “(C₃-C₈)cycloalkyl” refers to a non-aromatic cyclic hydrocarbon ring having from three to eight ring carbon atoms. Exemplary “(C₃-C₈)cycloalkyl” groups useful in the present invention include cyclopropyl, cyclobutyl, cyclopentyl, cyclohexyl, cycloheptyl, and cyclooctyl.

“Alkoxy” means an alkyl radical containing the specified number of carbon atoms attached through an oxygen linking atom. The term “(C₁-C₄)alkoxy” refers to a straight- or branched-chain hydrocarbon radical having at least 1 and up to 4 carbon atoms attached through an oxygen linking atom. Exemplary “(C₁-C₄)alkoxy” groups useful in the present invention include, but are not limited to, methoxy, ethoxy, n-propoxy, isopropoxy, n-butoxy, s-butoxy, and t-butoxy.

“Aryl” represents a group or moiety comprising an aromatic, monovalent monocyclic or bicyclic hydrocarbon radical containing from 6 to 10 carbon ring atoms, to which may be fused one or more cycloalkyl rings.

Generally, in the compounds of this invention, aryl is phenyl.

Heterocyclic groups may be heteroaryl or heterocycloalkyl groups.

“Heteroaryl” represents a group or moiety comprising an aromatic monovalent monocyclic or bicyclic radical, containing 5 to 10 ring atoms, including 1 to 4 heteroatoms independently selected from nitrogen, oxygen and sulfur. This term also encompasses bicyclic heterocyclic-aryl compounds containing an aryl ring moiety fused to a heterocycloalkyl ring moiety, containing 5 to 10 ring atoms, including 1 to 4 heteroatoms independently selected from nitrogen, oxygen and sulfur. Illustrative examples of heteroaryls useful in the present invention include, but are not limited to, furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, triazinyl, benzofuranyl, isobenzofuryl, 2,3-dihydrobenzofuryl, 1,3-benzodioxolyl, dihydrobenzodioxinyl, benzothienyl, indolizinyl, indolyl, isoindolyl, dihydroindolyl, benzimidazolyl, dihydrobenzimidazolyl, benzoxazolyl, dihydrobenzoxazolyl, benzthiazolyl, benzoisothiazolyl, dihydrobenzoisothiazolyl, indazolyl, imidazopyridinyl, pyrazolopyridinyl, benzotriazolyl, triazolopyridinyl, purinyl, quinolinyl, tetrahydroquinolinyl, isoquinolinyl, tetrahydroisoquinolinyl, quinoxalinyl, cinnolinyl, phthalazinyl, quinazolinyl, 1,5-naphthyridinyl, 1,6-naphthyridinyl, 1,7-naphthyridinyl, 1,8-naphthyridinyl, and pteridinyl.

Generally, the heteroaryl groups present in the compounds of this invention are 5-membered and/or 6-membered monocyclic heteroaryl groups. Selected 5-membered heteroaryl groups contain one nitrogen, oxygen, or sulfur ring heteroatom, and optionally contain 1, 2, or 3 additional nitrogen ring atoms. Selected 6-membered heteroaryl groups contain 1, 2, or 3 nitrogen ring heteroatoms. Illustrative examples of 5- or 6-membered heteroaryl groups useful in the present invention include, but are not limited to furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, and triazinyl.

“Heterocycloalkyl” represents a group or moiety comprising a non-aromatic, monovalent monocyclic or bicyclic radical, which is saturated or partially unsaturated, containing 3 to 10 ring atoms, which includes 1 to 3 heteroatoms independently selected from nitrogen, oxygen and sulfur. Illustrative examples of heterocycloalkyls useful in the present invention include, but are not limited to, azetidinyl, pyrrolidinyl, pyrazolidinyl, pyrazolinyl, imidazolidinyl, imidazolinyl, oxazolinyl, thiazolinyl, tetrahydrofuranyl, dihydrofuranyl, 1,3-dioxolanyl, piperidinyl, piperazinyl, morpholinyl, thiomorpholinyl, tetrahydropyranyl, dihydropyranyl, 1,3-dioxanyl, 1,4-dioxanyl, 1,3-oxathiolanyl, 1,3-oxathianyl, 1,3-dithianyl, hexahydro-1H-1,4-diazepinyl, azabicylo[3.2.1]octyl, azabicylo[3.3.1]nonyl, azabicylo[4.3.0]nonyl, oxabicylo[2.2.1]heptyl and 1,5,9-triazacyclododecyl.

Generally, in the compounds of this invention, heterocycloalkyl groups are 5-7 membered heterocycloalkyl groups, such as pyrrolidinyl, pyrazolidinyl, pyrazolinyl, imidazolidinyl, imidazolinyl, oxazolinyl, thiazolinyl, tetrahydrofuranyl, dihydrofuranyl, 1,3-dioxolanyl, piperidinyl, piperazinyl, morpholinyl, thiomorpholinyl, tetrahydropyranyl, dihydropyranyl, and hexahydro-1H-1,4-diazepinyl.

“Oxo” represents a double-bonded oxygen moiety; for example, if attached directly to a carbon atom forms a carbonyl moiety (C═O).

The terms “halogen” and “halo” represent chloro, fluoro, bromo, or iodo substituents. “Hydroxy” or “hydroxyl” is intended to mean the radical —OH.

“RORγ” refers to all isoforms encoded by the RORC gene which include RORγ 1 and RORγt.

“RORγ modulator” refers to a chemical compound that inhibits, either directly or indirectly, the activity of RORγ. RORγ modulators include antagonists and inverse agonists of RORγ.

“Pharmaceutically acceptable” refers to those compounds, materials, compositions, and dosage forms which are, within the scope of sound medical judgment, suitable for use in contact with the tissues of human beings and animals without excessive toxicity, irritation, or other problem or complication, commensurate with a reasonable benefit/risk ratio.

As used herein, the term “pharmaceutically acceptable salts” refers to salts that retain the desired biological activity of the subject compound and exhibit minimal undesired toxicological effects. These pharmaceutically acceptable salts may be prepared in situ during the final isolation and purification of the compound, or by separately reacting the purified compound in its free acid or free base form with a suitable base or acid, respectively.

As used herein, the term “compound(s) of the invention” means a compound of Formula (I) (as defined above) in any form, i.e., any salt or non-salt form (e.g., as a free acid or base form, or as a pharmaceutically acceptable salt thereof) and any physical form thereof (e.g., including non-solid forms (e.g., liquid or semi-solid forms), and solid forms (e.g., amorphous or crystalline forms, specific polymorphic forms, solvates, including hydrates (e.g., mono-, di- and hemi-hydrates)), and mixtures of various forms.

As used herein, the term “optionally substituted” indicates that a group, such as alkyl, cycloalkyl, alkoxy, heterocycloalkyl, aryl, or heteroaryl, may be unsubstituted, or the group may be substituted with one or more substituent(s) as defined. In the case where groups may be selected from a number of alternative groups the selected groups may be the same or different.

The term “independently” means that where more than one substituent is selected from a number of possible substituents, those substituents may be the same or different. The alternative definitions for the various groups and substituent groups of Formula (I) provided throughout the specification are intended to particularly describe each compound species disclosed herein, individually, as well as groups of one or more compound species. The scope of this invention includes any combination of these group and substituent group definitions.

Suitably, m is 0, 1, or 2. In a specific embodiment of this invention, m is 1.

Suitably, n is 0, 1, 2, or 3. In another embodiment of this invention, n is 1 or 2.

Suitably, X¹, X², X³, X⁴, and X⁵ are each independently selected from N, N⁺—O⁻ (i.e. N-oxide), CH, and CR⁵, wherein 0-3 of X¹, X², X³, X⁴, and X⁵ are N or N⁺—O⁻ and 1-3 of X¹, X², X³, X⁴, and X⁵ are CR⁵; provided that when zero of X¹, X², X⁴, and X⁵ are N or N⁺—O⁻ and X³ is CR⁵, 1-2 of X¹, X², X⁴, and X⁵ are CR⁵. In another embodiment of this invention, X¹, X², X³, X⁴, and X⁵ are each independently selected from N, N⁺—O⁻, CH, and CR⁵, wherein 0-2 of X¹, X², X³, X⁴, and X⁵ are N or N⁺—O⁻ and 1-3 of X¹, X², X³, X⁴, and X⁵ are CR⁵; provided that when zero of X¹, X², X⁴, and X⁵ are N or N⁺—O⁻ and X³ is CR⁵, 1-2 of X¹, X², X⁴, and X⁵ are CR⁵. In another embodiment of this invention, X¹ and X⁵ are each independently selected from N, N⁺—O⁻, CH, and CR⁵, and X², X³, and X⁴ are each independently selected from CH and CR⁵, wherein at least one of X¹ and X⁵ is N or N⁺—O⁻ and 0-3 of X¹, X², X³, X⁴, and X⁵ are CR⁵. In another embodiment of this invention, X¹ and X⁵ are each independently selected from N, N⁺—O⁻, and a carbon atom substituted by hydrogen, halogen, cyano, (C₁-C₄)alkyl, (C₁-C₄)haloalkyl, (C₁-C₄)alkoxy, or ((C₁-C₄)alkyl)((C₁-C₄)alkyl)amino (i.e. N, N⁺—O⁻, CH, and CR⁵, wherein R⁵ is halogen, cyano, (C₁-C₄)alkyl, (C₁-C₄)haloalkyl, (C₁-C₄)alkoxy, or ((C₁-C₄)alkyl)((C₁-C₄)alkyl)amino), and X², X³, and X⁴ are each independently a carbon atom substituted by hydrogen, halogen, cyano, (C₁-C₄)alkyl, (C₁-C₄)haloalkyl, (C₁-C₄)alkoxy, or ((C₁-C₄)alkyl)((C₁-C₄)alkyl)amino (i.e. CH or CR⁵, wherein R⁵ is halogen, cyano, (C₁-C₄)alkyl, (C₁-C₄)haloalkyl, (C₁-C₄)alkoxy, or ((C₁-C₄)alkyl)((C₁-C₄)alkyl)amino), wherein at least one of X¹ and X⁵ is N or N⁺—O⁻ and 2-4 of X¹, X², X³, X⁴, and X⁵ are a carbon atom substituted by hydrogen (i.e. CH). In another embodiment of this invention, X² is N or N⁺—O⁻, and X¹, X³, X⁴, and X⁵ are each independently a carbon atom substituted by hydrogen, halogen, cyano, (C₁-C₄)alkyl, (C₁-C₄)haloalkyl, (C₁-C₄)alkoxy, or ((C₁-C₄)alkyl)((C₁-C₄)alkyl)amino, wherein 2-4 of X¹, X³, X⁴, and X⁵ are a carbon atom substituted by hydrogen. In another embodiment of this invention, X¹, X², X³. X⁴, and X⁵ are each independently selected from CH and CR⁵, wherein 0-3 of X¹, X², X³, X⁴, and X⁵ are CR⁵. In another embodiment of this invention, X¹, X², X³, X⁴, and X⁵ are each independently a carbon atom substituted by hydrogen, halogen, cyano, (C₁-C₄)alkyl, (C₁-C₄)haloalkyl, (C₁-C₄)alkoxy, or ((C₁-C₄)alkyl)((C₁-C₄)alkyl)amino, wherein 2-5 of X¹, X², X³, X⁴, and X⁵ are a carbon atom substituted by hydrogen. In another embodiment of this invention, X¹ is a carbon atom substituted by halogen, (C₁-C₄)alkyl, (C₁-C₄)haloalkyl, cyano, (C₁-C₄)alkoxy, or ((C₁-C₄)alkyl)((C₁-C₄)alkyl)amino, and X², X³, X⁴, and X⁵ are each independently a carbon atom substituted by hydrogen, halogen, (C₁-C₄)alkyl, (C₁-C₄)haloalkyl, cyano, (C₁-C₄)alkoxy, or ((C₁-C₄)alkyl)((C₁-C₄)alkyl)amino, wherein 2-4 of X², X³, X⁴, and X⁵ are a carbon atom substituted by hydrogen.

Suitably, one of Y¹ and Y² is O or NR⁸ and the other is a bond. In another embodiment of this invention, one of Y¹ and Y² is O, NH, or N((C₁-C₄)alkyl) and the other is a bond. In a specific embodiment of this invention, Y¹ is NH or NCH₃ and Y² is a bond. In another specific embodiment of this invention, Y¹ is NH and Y² is a bond. In another specific embodiment of this invention, Y¹ is a bond and Y² is NH.

In another embodiment of this invention, X¹ is CR⁵, Y¹ is NR⁸, Y² is a bond, and R⁵ and R⁸ taken together with the atoms to which they are attached form a five to seven membered ring, optionally containing an additional heteroatom selected from oxygen, nitrogen, and sulfur, which ring is optionally substituted by (C₁-C₄)alkyl. In another embodiment of this invention, X¹ is CR⁵, Y¹ is NR⁸, Y² is a bond, and R⁵ and R⁸ taken together represent —CH₂—, —CH₂CH₂—, or —CH₂CH₂CH₂—.

Suitably, Cy is (C₃-C₈)cycloalkyl, heterocycloalkyl, phenyl, or 5- or 6-membered heteroaryl, each of which is optionally substituted one, two, or three times, independently, by (C₁-C₆)alkyl, (C₁-C₆)haloalkyl, (C₃-C₆)cycloalkyl, halogen, oxo, cyano, hydroxyl, hydroxy(C₁-C₆)alkyl, (C₁-C₆)alkoxy, —((C₀-C₃)alkyl)NHCO₂R⁷, —((C₀-C₃)alkyl)N((C₁-C₄)alkyl)CO₂R⁷, —((C₀-C₃)alkyl)NHC(O)R⁷, —((C₀-C₃)alkyl)N((C₁-C₄)alkyl)C(O)R⁷, —((C₀-C₃)alkyl)CO₂R⁷, —((C₀-C₃)alkyl)CONR⁷R⁸, —((C₀-C₃)alkyl)C(O)R⁷, (C₁-C₄)alkoxy(C₁-C₆)alkyl, amino(C₁-C₆)alkyl, ((C₁-C₄)alkyl)((C₁-C₄)alkyl)amino(C₁-C₆)alkyl, (C₁-C₄)alkylamino(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, or heterocycloalkyl. In another embodiment of this invention, Cy is heterocycloalkyl, phenyl, or 5- or 6-membered heteroaryl, each of which is optionally substituted one or two times, independently, by (C₁-C₆)alkyl, (C₁-C₆)haloalkyl, halogen, cyano, (C₁-C₄)alkoxy, ((C₁-C₄)alkyl)amino ((C₁-C₄)alkyl)((C₁-C₄)alkyl)amino, —((C₀-C₃)alkyl)CO₂R⁷, or —((C₀-C₃)alkyl)CONR⁷R⁸. In another embodiment of this invention, Cy is (C₃-C₆)cycloalkyl, azetidinyl, pyrrolidinyl, pyrazolidinyl, pyrazolinyl, imidazolidinyl, imidazolinyl, oxazolinyl, thiazolinyl, tetrahydrofuranyl, dihydrofuranyl, piperidinyl, piperazinyl, morpholinyl, thiomorpholinyl, tetrahydropyranyl, dihydropyranyl, dioxanyl, oxathianyl, phenyl, furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, or triazinyl, each of which is optionally substituted one, two, or three times, independently, by (C₁-C₆)alkyl, (C₁-C₆)haloalkyl, (C₃-C₆)cycloalkyl, halogen, oxo, cyano, hydroxyl, hydroxy(C₁-C₆)alkyl, (C₁-C₆)alkoxy, —((C₀-C₃)alkyl)NHCO₂R⁷, —((C₀-C₃)alkyl)N((C₁-C₄)alkyl)CO₂R⁷, —((C₀-C₃)alkyl)NHC(O)R⁷, —((C₀-C₃)alkyl)N((C₁-C₄)alkyl)C(O)R⁷, —((C₀-C₃)alkyl)CO₂R⁷, —((C₀-C₃)alkyl)CONR⁷R⁸, —((C₀-C₃)alkyl)C(O)R⁷, (C₁-C₄)alkoxy(C₁-C₆)alkyl, amino(C₁-C₆)alkyl, ((C₁-C₄)alkyl)((C₁-C₄)alkyl)amino(C₁-C₆)alkyl, (C₁-C₄)alkylamino(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, or heterocycloalkyl. In another embodiment of this invention, Cy is piperidinyl, piperazinyl, phenyl, pyridinyl, pyridazinyl, pyrazinyl, or pyrimidinyl, each of which is optionally substituted one, two, or three times, independently, by (C₁-C₆)alkyl, (C₁-C₆)haloalkyl, (C₃-C₆)cycloalkyl, halogen, oxo, cyano, hydroxyl, hydroxy(C₁-C₆)alkyl, (C₁-C₆)alkoxy, —((C₀-C₃)alkyl)NHCO₂R⁷, —((C₀-C₃)alkyl)N((C₁-C₄)alkyl)CO₂R⁷, —((C₀-C₃)alkyl)NHC(O)R⁷, —((C₀-C₃)alkyl)N((C₁-C₄)alkyl)C(O)R⁷, —((C₀-C₃)alkyl)CO₂R⁷, —((C₀-C₃)alkyl)CONR⁷R⁸, —((C₀-C₃)alkyl)C(O)R⁷, (C₁-C₄)alkoxy(C₁-C₆)alkyl, amino(C₁-C₆)alkyl, ((C₁-C₄)alkyl)((C₁-C₄)alkyl)amino(C₁-C₆)alkyl, (C₁-C₄)alkylamino(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, or heterocycloalkyl. In another embodiment of this invention, Cy is piperidinyl, piperazinyl, phenyl, pyridinyl, pyridazinyl, pyrazinyl, or pyrimidinyl, each of which is optionally substituted one or two times, independently, by (C₁-C₆)alkyl, (C₁-C₆)haloalkyl, halogen, cyano, (C₁-C₄)alkoxy, (C₁-C₄)alkyl)((C₁-C₄)alkyl)amino, —((C₀-C₃)alkyl)CO₂H, —((C₀-C₃)alkyl)CO₂(C₁-C₆)alkyl, or —((C₀-C₃)alkyl)CONH(C₁-C₆)alkyl. In another embodiment of this invention, Cy is phenyl, which is optionally substituted one, two, or three times, independently, by (C₁-C₆)alkyl, (C₁-C₆)haloalkyl, (C₃-C₆)cycloalkyl, halogen, oxo, cyano, hydroxyl, hydroxy(C₁-C₆)alkyl, (C₁-C₆)alkoxy, —((C₀-C₃)alkyl)NHCO₂R⁷, —((C₀-C₃)alkyl)N((C₁-C₄)alkyl)CO₂R⁷, —((C₀-C₃)alkyl)NHC(O)R⁷, —((C₀-C₃)alkyl)N((C₁-C₄)alkyl)C(O)R⁷, —((C₀-C₃)alkyl)CO₂R⁷, —((C₀-C₃)alkyl)CONR⁷R⁸, —((C₀-C₃)alkyl)C(O)R⁷, (C₁-C₄)alkoxy(C₁-C₆)alkyl, amino(C₁-C₆)alkyl, ((C₁-C₄)alkyl)((C₁-C₄)alkyl)amino(C₁-C₆)alkyl, (C₁-C₄)alkylamino(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, or heterocycloalkyl. In another embodiment of this invention, Cy is phenyl, which is optionally substituted one or two times, independently, by halogen, (C₁-C₄)alkyl, (C₁-C₄)haloalkyl, cyano, (C₁-C₄)alkoxy, —((C₀-C₃)alkyl)CO₂R⁷, or —((C₀-C₃)alkyl)CONR⁷R⁸ or ((C₁-C₄)alkyl)((C₁-C₄)alkyl)amino. In another embodiment of this invention, Cy is phenyl, which is optionally substituted one or two times, independently, by halogen, (C₁-C₄)alkyl, (C₁-C₄)haloalkyl, cyano, (C₁-C₄)alkoxy, or ((C₁-C₄)alkyl)((C₁-C₄)alkyl)amino. In a specific embodiment of this invention, Cy is phenyl.

Suitably, Z is O, S, SO₂, C═O, NR⁶, or a bond. In another embodiment of this invention, Z is O, NR⁶, or a bond. In another embodiment of this invention, Z is O, NH, —N(C₁-C₄)alkyl, —N((C₀-C₃)alkyl)CO₂R⁷, —N((C₀-C₃)alkyl)CONR⁷R⁸ or a bond. In another embodiment of this invention, Z is a bond, O, or NH. In another embodiment of this invention, Z is O or NH. In a specific embodiment of this invention, Z is O.

Suitably, A¹, A², A³, and A⁴ are each independently selected from N, NR⁶, O, S, CH, and CR¹⁰, wherein one of A¹, A², A³, and A⁴ is NR⁶, O, or S, 0-2 of A¹, A², A³, and A⁴ are CR¹⁰, and 0-3 of A¹, A², A³, and A⁴ are CH or N. In another embodiment of this invention, A¹, A², A³, and A⁴ are each independently selected from N, N((C₁-C₄)alkyl), O, S, CH, and C((C₁-C₄)alkyl), wherein one of A¹, A², A³, and A⁴ is N((C₁-C₄)alkyl), O, or S, 0-2 of A¹, A², A³, and A⁴ are C((C₁-C₄)alkyl), and 0-3 of A¹, A², A³, and A⁴ are CH or N. In another embodiment of this invention, A¹ and A⁴ are each independently selected from CH and CR¹⁰, and one of A² and A³ is NR⁶, O, or S and the other is N or CH. In another embodiment of this invention. A¹ and A⁴ are each independently selected from CH and C((C₁-C₄)alkyl), and one of A² and A³ is N((C₁-C₄)alkyl), O, or S and the other is N or CH. In another embodiment of this invention, A¹ and A⁴ are each independently selected from CH and C((C₁-C₄)alkyl), and one of A² and A³ is O or S and the other is N.

Suitably, R¹ is (C₃-C₆)alkyl, (C₃-C₆)haloalkyl, (C₃-C₈)cycloalkyl, (C₃-C₆)alkoxy, (C₁-C₆)alkoxy(C₁-C₂)alkyl, aryl, heteroaryl, aryl(C₁-C₆)alkyl, heteroaryl(C₁-C₆)alkyl, or heterocycloalkyl, each of which is optionally substituted one, two, or three times, independently, by R⁵. In another embodiment of this invention. R¹ is (C₃-C₆)alkyl, (C₃-C₈)cycloalkyl, (C₁-C₆)alkoxy(C₁-C₂)alkyl, aryl, or heteroaryl, each of which is optionally substituted one, two, or three times, independently, by R⁵. In another embodiment of this invention, R¹ is (C₃-C₆)alkyl, (C₃-C₆)cycloalkyl, (C₁-C₆)alkoxy(C₁-C₂)alkyl, phenyl, furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, or triazinyl, wherein said phenyl, furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, or triazinyl is optionally substituted one or two times, independently, by halogen, (C₁-C₄)alkyl, (C₁-C₄)haloalkyl, cyano, (C₁-C₄)alkoxy, or ((C₁-C₄)alkyl)((C₁-C₄)alkyl)amino (i.e. wherein R⁵ is halogen, (C₁-C₄)alkyl, (C₁-C₄)haloalkyl, cyano, (C₁-C₄)alkoxy, or ((C₁-C₄)alkyl)((C₁-C₄)alkyl)amino). In another embodiment of this invention, R¹ is (C₃-C₆)alkyl, (C₃-C₆)cycloalkyl, phenyl, furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, or triazinyl, wherein said phenyl, furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, or triazinyl is optionally substituted one or two times, independently, by halogen, (C₁-C₄)alkyl, (C₁-C₄)alkoxy, or ((C₁-C₄)alkyl)((C₁-C₄)alkyl)amino. In another embodiment of this invention, R¹ is (C₃-C₆)alkyl. In another embodiment of this invention, R¹ is (C₁-C₆)alkyl. In another embodiment of this invention, R¹ is phenyl or pyridinyl, each of which is optionally substituted one or two times, independently, by halogen, (C₁-C₄)alkyl, (C₁-C₄)haloalkyl, cyano, (C₁-C₄)alkoxy, or ((C₁-C₄)alkyl)((C₁-C₄)alkyl)amino. In another embodiment of this invention, R¹ is phenyl or pyridinyl, each of which is optionally substituted one or two times, independently, by halogen, (C₁-C₄)alkyl, (C₁-C₄)alkoxy, or ((C₁-C₄)alkyl)((C₁-C₄)alkyl)amino. In another embodiment of this invention, R¹ is phenyl optionally substituted one or two times, independently, by halogen, (C₁-C₄)alkyl, (C₁-C₄)haloalkyl, cyano, (C₁-C₄)alkoxy, or ((C₁-C₄)alkyl)((C₁-C₄)alkyl)amino. In a specific embodiment of this invention, R¹ is phenyl or pyridinyl. In another specific embodiment of this invention, R¹ is phenyl.

Suitably, R² is hydrogen, (C₁-C₆)alkyl, or (C₁-C₆)haloalkyl. In another embodiment of this invention, hydrogen or (C₁-C₄)alkyl. In another embodiment of this invention, R² is hydrogen or methyl. In a specific embodiment of this invention, R² is hydrogen.

In another embodiment of this invention, R¹ and R² taken together with the carbon atom to which they are attached form a three to eight membered ring, optionally containing a heteroatom selected from oxygen, nitrogen, and sulfur, which ring is optionally substituted one, two, or three times, independently, by R⁵. In another embodiment of this invention, R¹ and R² taken together represent —CH₂CH₂CH—, —CH₂CH₂CH_CH₂—, or —CH₂CH₂CH₂CH₂CH₂—.

Suitably, R³ and R^{3a} are each independently hydrogen, hydroxyl, (C₁-C₆)alkyl, (C₁-C₆)haloalkyl, halogen, (C₁-C₆)alkoxy, amino, (C₁-C₄)alkylamino, or ((C₁-C₄)alkyl)((C₁-C₄)alkyl)amino. In another embodiment of this invention, R³ and R^{3a} are each independently hydrogen or methyl. In a specific embodiment of this invention. R³ and R^{3a} are each independently hydrogen.

Suitably, each R⁴ is independently selected from hydrogen, halogen, (C₁-C₆)alkyl, (C₁-C₆)haloalkyl, —CO₂R⁷, —CONR⁷R⁸, —OR⁹, and —NR⁸R⁹, wherein said (C₁-C₆)alkyl or (C₁-C₆)haloalkyl is optionally substituted by hydroxyl, —OR⁹, —CO₂R⁷, —CONR⁷R⁸, or —NR⁸R⁹. In another embodiment of this invention, each R⁴ is independently selected from hydrogen, (C₁-C₄)alkyl, (C₁-C₄)haloalkyl, —OR⁹, and —NR⁸R⁹, wherein said (C₁-C₄)alkyl or (C₁-C₄)haloalkyl is optionally substituted by hydroxyl, —OR⁹, —CO₂R⁷, —CONR⁷R⁸, or —NR⁸R⁹. In another embodiment of this invention, each R⁴ is independently selected from hydrogen, halogen, (C₁-C₄)alkyl, (C₁-C₄)alkylamino, ((C₁-C₄)alkyl)((C₁-C₄)alkyl)amino, (C₁-C₄)alkoxy(C₁-C₄)alkylamino, —NHCO₂(C₁-C₄)alkyl, (C₁-C₄)alkoxy, hydroxy(C₂-C₄)alkoxy, (C₁-C₄)alkoxy(C₂-C₄)alkoxy, amino(C₂-C₄)alkoxy, —O((C₁-C₄)alkyl)CO₂R⁷, —O((C₁-C₄)alkyl)CONH₂. —O((C₁-C₄)alkyl)CONH(C₁-C₄)alkyl, —O((C₁-C₄)alkyl)CON((C₁-C₄)alkyl)((C₁-C₄)alkyl), and CO₂R⁷. In another embodiment of this invention, each R⁴ is independently selected from hydrogen, halogen, (C₁-C₄)alkyl, (C₁-C₄)alkylamino, ((C₁-C₄)alkyl)((C₁-C₄)alkyl)amino, (C₁-C₄)alkoxy(C₁-C₄)alkylamino, (C₁-C₄)alkoxy, hydroxy(C₂-C₄)alkoxy, (C₁-C₄)alkoxy(C₂-C₄)alkoxy, amino(C₂-C₄)alkoxy, —O((C₁-C₃)alkyl)CO₂H, —O((C₁-C₃)alkyl)CO₂(C₁-C₄)alkyl, —O((C₁-C₃)alkyl)CONH₂, —O((C₁-C₃)alkyl)CONH(C₁-C₄)alkyl, and —O((C₁-C₃)alkyl)CON((C₁-C₄)alkyl)((C₁-C₄)alkyl). In another embodiment of this invention, each R⁴ is independently selected from hydrogen, (C₁-C₄)alkyl, (C₁-C₄)alkoxy, hydroxy(C₂-C₄)alkoxy, (C₁-C₄)alkoxy(C₂-C₄)alkoxy, amino(C₂-C₄)alkoxy, —O((C₁-C₃)alkyl)CO₂H, —O((C₁-C₃)alkyl)CO₂(C₁-C₄)alkyl, —O((C₁-C₃)alkyl)CONH₂. —O((C₁-C₃)alkyl)CONH(C₁-C₄)alkyl, and —O((C₁-C₃)alkyl)CON((C₁-C₄)alkyl)((C₁-C₄)alkyl). In another embodiment of this invention, each R⁴ is independently selected from (C₁-C₄)alkoxy, hydroxy(C₂-C₄)alkoxy, (C₁-C₄)alkoxy(C₂-C₄)alkoxy, amino(C₂-C₄)alkoxy, —O((C₁-C₃)alkyl)CO₂H, —O((C₁-C₃)alkyl)CO₂(C₁-C₄)alkyl, —O((C₁-C₃)alkyl)CONH₂, —O((C₁-C₃)alkyl)CONH(C₁-C₄)alkyl, and —O((C₁-C₃)alkyl)CON((C₁-C₄)alkyl)((C₁-C₄)alkyl). In another embodiment of this invention, each R⁴ is independently selected from (C₁-C₄)alkoxy, —O((C₁-C₃)alkyl)CO₂H, —O((C₁-C₃)alkyl)CO₂(C₁-C₄)alkyl, —O((C₁-C₃)alkyl)CONH₂, —O((C₁-C₃)alkyl)CONH(C₁-C₆)alkyl, and —O((C₁-C₃)alkyl)CON((C₁-C₄)alkyl)((C₁-C₄)alkyl). In another embodiment of this invention, each R⁴ is independently selected from (C₁-C₄)alkyl and (C₁-C₄)alkoxy. In a specific embodiment of this invention, each R⁴ is hydrogen.

Suitably, each R^{4a} is independently selected from hydrogen, halogen, hydroxyl, amino, and (C₁-C₆)alkyl. In another embodiment of this invention, each R^{4a} is independently selected from hydrogen, halogen, and (C₁-C₄)alkyl. In another embodiment of this invention, each R^{4a} is independently selected from is hydrogen, fluorine, and methyl. In another embodiment of this invention, each R^{4a} is independently selected from is hydrogen and methyl. In a specific embodiment of this invention, each R^{4a} is hydrogen. In a specific embodiment of this invention, each R^{4a} is methyl.

In another embodiment of this invention, R⁴ and R^{4a} taken together with the carbon atom to which they are attached form a three to eight membered ring, optionally containing a heteroatom selected from oxygen, nitrogen, and sulfur, which ring is optionally substituted by cyano, (C₁-C₄)alkyl, (C₁-C₄)haloalkyl, (C₃-C₆)cycloalkyl, —CO₂R⁷, —CONR⁷R⁸, hydroxyl, hydroxy(C₁-C₆)alkyl, (C₁-C₄)alkoxy, (C₁-C₄)alkoxy(C₁-C₆)alkyl, amino, (C₁-C₄)alkylamino, ((C₁-C₄)alkyl)((C₁-C₄)alkyl)amino, —NHCO₂R⁷, —N((C₁-C₄)alkyl)CO₂R⁷, —NHC(O)R⁷, or —N((C₁-C₄)alkyl)C(O)R⁷. In another embodiment of this invention, R⁴ and R^{4a} taken together represent —CH₂CH₂—. —CH₂CH₂CH₂—. —CH₂CH₂CH₂CH₂—, or —CH₂CH₂CH₂CH₂CH₂—.

One particular embodiment of the invention is a compound of Formula (Ia):

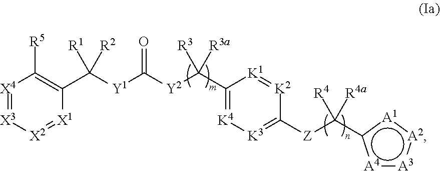

wherein:

m is 1;

n is 1 or 2;

X¹, X², X³, and X⁴ are each independently selected from N, N⁺—O⁻, CH, and CR⁵, wherein 0-2 of X¹, X², X³, and X⁴ are N or N⁺—O⁻ and 0-2 X¹, X², X³, and X⁴ are CR⁵;

Y¹ is NH or NCH₃ and Y² is a bond;

K¹, K², K³, and K⁴ are each independently selected from N, N⁺—O⁻, CH, and CR¹⁰, wherein 0-2 of K¹, K², K³, and K⁴ are N or N⁺—O⁻ and 0-2 of K¹, K², K³, and K⁴ are CR¹⁰;

Z is O, NR⁶, or a bond;

A¹, A², A³, and A⁴ are each independently selected from N, NR⁶, O, S, CH, and CR¹⁰, wherein one of A¹, A², A³, and A⁴ is NR⁶, O, or S, 0-2 of A¹, A², A³, and A⁴ are CR¹⁰, and 0-3 of A¹, A², A³, and A⁴ are CH or N;

R¹ is (C₃-C₆)alkyl, (C₃-C₆)haloalkyl, (C₃-C₈)cycloalkyl, (C₃-C₆)alkoxy, (C₁-C₆)alkoxy(C₁-C₂)alkyl, aryl, heteroaryl, aryl(C₁-C₆)alkyl, heteroaryl(C₁-C₆)alkyl, or heterocycloalkyl, each of which is optionally substituted one, two, or three times, independently, by R⁵;

R² is hydrogen, (C₁-C₆)alkyl, or (C₁-C₆)haloalkyl;

or R¹ and R² taken together with the carbon atom to which they are attached form a three to eight membered ring, optionally containing a heteroatom selected from oxygen, nitrogen, and sulfur, which ring is optionally substituted one, two, or three times, independently, by R⁵;

R³ and R^{3a} are each independently hydrogen, hydroxyl, (C₁-C₄)alkyl, (C₁-C₄)haloalkyl, halogen, (C₁-C₄)alkoxy, amino, (C₁-C₄)alkylamino, or ((C₁-C₄)alkyl)((C₁-C₄)alkyl)amino;

each R⁴ is independently selected from hydrogen, halogen, (C₁-C₄)alkyl, (C₁-C₄)haloalkyl, —OR⁹, and —NR⁸R⁹, wherein said (C₁-C₄)alkyl or (C₁-C₄)haloalkyl is optionally substituted by hydroxyl, —OR⁹, —CO₂R⁷, —CONR⁷R⁸, or —NR⁸R⁹;

each R^{4a} is independently selected from hydrogen, halogen, hydroxyl, amino, and (C₁-C₄)alkyl;

each R⁵ is independently selected from (C₁-C₆)alkyl, (C₁-C₆)haloalkyl, (C₁-C₆)cycloalkyl, halogen, cyano, hydroxyl, hydroxy(C₁-C₆)alkyl, (C₁-C₆)alkoxy, (C₁-C₄)alkoxy(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, and heterocycloalkyl;

R⁶ is hydrogen, (C₁-C₆)alkyl, (C₁-C₆)haloalkyl, (C₃-C₆)cycloalkyl, hydroxy(C₁-C₆)alkyl, (C₁-C₄)alkoxy(C₁-C₆)alkyl, —((C₀-C₃)alkyl)CO₂R⁷, —((C₀-C₃)alkyl)CONR⁷R⁸, aryl, heteroaryl, aryl(C₁-C₆)alkyl, heteroaryl(C₁-C₆)alkyl, or heterocycloalkyl;

R⁷ is hydrogen, (C₁-C₆)alkyl, (C₁-C₆)haloalkyl, (C₃-C₆)cycloalkyl, (C₁-C₄)alkoxy(C₁-C₆)alkyl, aryl, heteroaryl, aryl(C₁-C₆)alkyl, heteroaryl(C₁-C₆)alkyl, or heterocycloalkyl;

R⁸ is hydrogen, (C₁-C₆)alkyl, or (C₁-C₆)haloalkyl;

or R⁷ and R⁸ taken together with the nitrogen atom to which they are attached form a four to eight membered ring, optionally containing an additional heteroatom selected from oxygen, nitrogen, and sulfur, which ring is optionally substituted by (C₁-C₄)alkyl, (C₁-C₄)haloalkyl, (C₃-C₆)cycloalkyl, —CO₂H, —CO₂(C₁-C₄)alkyl, hydroxyl, hydroxy(C₁-C₆)alkyl, (C₁-C₄)alkoxy, (C₁-C₄)alkoxy(C₁-C₆)alkyl, amino, (C₁-C₄)alkylamino, or ((C₁-C₄)alkyl)((C₁-C₄)alkyl)amino;

R⁹ is —C(O)R⁷, —CO₂R⁷, —C(O)NR⁷R⁸, (C₁-C₆)alkyl, (C₁-C₆)haloalkyl, (C₃-C₆)cycloalkyl, aryl, heteroaryl, aryl(C₁-C₆)alkyl, heteroaryl(C₁-C₆)alkyl, or heterocycloalkyl, wherein said (C₁-C₆)alkyl, (C₁-C₆)haloalkyl, (C₃-C₆)cycloalkyl, aryl, heteroaryl, aryl(C₁-C₆)alkyl, heteroaryl(C₁-C₆)alkyl, or heterocycloalkyl is optionally substituted by —CO₂R⁷, —CONH₂, —CONH(C₁-C₄)alkyl, —CON((C₁-C₄)alkyl)((C₁-C₄)alkyl), hydroxyl, (C₁-C₄)alkoxy, amino, (C₁-C₄)alkylamino, ((C₁-C₄)alkyl)((C₁-C₄)alkyl)amino, —NHCO₂R⁷, —N((C₁-C₄)alkyl)CO₂R⁷, —NHC(O)R⁷, or —N((C₁-C₄)alkyl)C(O)R⁷;

or R⁸ and R⁹ taken together with the nitrogen atom to which they are attached form a four to eight membered ring, optionally containing an additional heteroatom selected from oxygen, nitrogen, and sulfur, which ring is optionally substituted by cyano, (C₁-C₄)alkyl, (C₁-C₄)haloalkyl, (C₃-C₆)cycloalkyl, —CO₂H, —CO₂(C₁-C₄)alkyl, —CONR⁷R⁸, hydroxyl, hydroxy(C₁-C₆)alkyl, (C₁-C₄)alkoxy, (C₁-C₄)alkoxy(C₁-C₆)alkyl, amino, (C₁-C₄)alkylamino, ((C₁-C₄)alkyl)((C₁-C₄)alkyl)amino, —NHCO₂R⁷, —N((C₁-C₄)alkyl)CO₂R⁷, —NHC(O)R⁷, or —N((C₁-C₄)alkyl)C(O)R⁷; and

R¹⁰ is (C₁-C₆)alkyl, (C₁-C₆)haloalkyl, (C₃-C₈)cycloalkyl, halogen, cyano, hydroxyl, hydroxy(C₁-C₆)alkyl, (C₁-C₆)alkoxy, (C₁-C₄)alkoxy(C₁-C₆)alkyl, —((C₀-C₃)alkyl)CO₂R⁷. —((C₀-C₃)alkyl)CONR⁷R⁸, amino(C₁-C₆)alkyl, ((C₁-C₄)alkyl)((C₁-C₄)alkyl)amino(C₁-C₆)alkyl, (C₁-C₄)alkylamino(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, or heterocycloalkyl;

or a pharmaceutically acceptable salt thereof.

Another particular embodiment of the invention is a compound of Formula (Ia) wherein:

m is 1;

n is 1 or 2;

X¹, X², X³, and X⁴ are each independently a carbon atom substituted by hydrogen, halogen, cyano, (C₁-C₄)alkyl, (C₁-C₄)haloalkyl, (C₁-C₄)alkoxy, or ((C₁-C₄)alkyl)((C₁-C₄)alkyl)amino, wherein 2-4 of X¹, X², X³, and X⁴ are a carbon atom substituted by hydrogen;

Y¹ is NH or NCH₃ and Y² is a bond;

K¹, K², K³, and K⁴ are each independently a carbon atom substituted by hydrogen, halogen, (C₁-C₄)alkyl, (C₁-C₄)alkoxy, or ((C₁-C₄)alkyl)((C₁-C₄)alkyl)amino, wherein 2-4 of K¹, K², K³, and K⁴ are a carbon atom substituted by hydrogen;

Z is O, NH, —N(C₁-C₄)alkyl, —N((C₀-C₃)alkyl)CO₂R⁷, —N((C₀-C₃)alkyl)CONR⁷R⁸, or a bond;

A¹ and A⁴ are each independently selected from CH and CR¹⁰, and one of A² and A³ is NR⁶, O, or S and the other is N or CH;

R¹ is (C₃-C₆)alkyl, (C₃-C₆)haloalkyl, (C₃-C₈)cycloalkyl, (C₃-C₆)alkoxy, (C₁-C₆)alkoxy(C₁-C₂)alkyl, aryl, heteroaryl, aryl(C₁-C₆)alkyl, heteroaryl(C₁-C₆)alkyl, or heterocycloalkyl, each of which is optionally substituted one, two, or three times, independently, by R⁵;

R² is hydrogen;

R³ and R^{3a} are each independently hydrogen or methyl;

each R⁴ is independently selected from hydrogen, (C₁-C₄)alkyl, (C₁-C₄)alkoxy, hydroxy(C₂-C₄)alkoxy, (C₁-C₄)alkylamino, ((C₁-C₄)alkyl)((C₁-C₄)alkyl)amino, (C₁-C₄)alkoxy(C₁-C₄)alkylamino, (C₁-C₄)alkoxy(C₂-C₄)alkoxy, amino(C₂-C₄)alkoxy, —O((C₁-C₃)alkyl)CO₂H, —O((C₁-C₃)alkyl)CO₂(C₁-C₄)alkyl, —O((C₁-C₃)alkyl)CONH₂, —O((C₁-C₃)alkyl)CONH(C₁-C₄)alkyl, and —O((C₁-C₃)alkyl)CON((C₁-C₄)alkyl)((C₁-C₄)alkyl);

each R^{4a} is independently selected from hydrogen, hydroxyl, amino, and (C₁-C₄)alkyl;

each R⁵ is independently selected from (C₁-C₆)alkyl, (C₁-C₆)haloalkyl, (C₃-C₆)cycloalkyl, halogen, cyano, hydroxyl, hydroxy(C₁-C₆)alkyl, (C₁-C₆)alkoxy, (C₁-C₄)alkoxy(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, and heterocycloalkyl;

R⁷ is hydrogen, (C₁-C₆)alkyl, (C₁-C₆)haloalkyl, (C₃-C₆)cycloalkyl, (C₁-C₄)alkoxy(C₁-C₆)alkyl, aryl, heteroaryl, aryl(C₁-C₆)alkyl, heteroaryl(C₁-C₆)alkyl, or heterocycloalkyl;

R⁸ is hydrogen, (C₁-C₆)alkyl, or (C₁-C₆)haloalkyl; and

R¹⁰ is (C₁-C₆)alkyl, (C₁-C₆)haloalkyl, (C₃-C₆)cycloalkyl, halogen, cyano, hydroxyl, hydroxy(C₁-C₆)alkyl, (C₁-C₆)alkoxy, (C₁-C₄)alkoxy(C₁-C₆)alkyl, —((C₀-C₃)alkyl)CO₂R⁷, —((C₀-C₃)alkyl)CONR⁷R⁸, amino(C₁-C₆)alkyl, ((C₁-C₄)alkyl)((C₁-C₄)alkyl)amino(C₁-C₆)alkyl, (C₁-C₄)alkylamino(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, or heterocycloalkyl;

or a pharmaceutically acceptable salt thereof.

Another particular embodiment of the invention is a compound of Formula (Ia) wherein:

m is 1;

n is 1 or 2;

X¹, X², X³, and X⁴ are each independently a carbon atom substituted by hydrogen, halogen, cyano, (C₁-C₄)alkyl, (C₁-C₄)haloalkyl, (C₁-C₄)alkoxy, or ((C₁-C₄)alkyl)((C₁-C₄)alkyl)amino, wherein 2-4 of X¹, X², X³, and X⁴ are a carbon atom substituted by hydrogen;

Y¹ is NH and Y² is a bond;

K¹, K², K³, and K⁴ are each independently a carbon atom substituted by hydrogen, halogen, (C₁-C₄)alkyl, (C₁-C₄)alkoxy, or ((C₁-C₄)alkyl)((C₁-C₄)alkyl)amino, wherein 2-4 of K¹, K², K³, and K⁴ are a carbon atom substituted by hydrogen;

Z is O, NH, —N(C₁-C₄)alkyl, or a bond;

A¹ and A⁴ are each independently selected from CH and C((C₁-C₄)alkyl), and one of A² and A³ is O or S and the other is N;

R¹ is phenyl optionally substituted one or two times, independently, by halogen, (C₁-C₄)alkyl, (C₁-C₄)haloalkyl, cyano, (C₁-C₄)alkoxy, or ((C₁-C₄)alkyl)((C₁-C₄)alkyl)amino;

R² is hydrogen;

R³ and R^{3a} are each independently hydrogen or methyl;

each R⁴ is independently selected from hydrogen, (C₁-C₄)alkyl, (C₁-C₄)alkylamino, ((C₁-C₄)alkyl)((C₁-C₄)alkyl)amino, and (C₁-C₄)alkoxy; and

each R^{4a} is independently selected from hydrogen, hydroxyl, amino, and (C₁-C₄)alkyl;

or a pharmaceutically acceptable salt thereof.

Specific compounds of this invention include:
N-((4-chloro-2-methylphenyl)(p-tolyl)methyl)-2-(4-((3,5-dimethylisoxazol-4-yl)methoxy)phenyl) acetamide;
N-((4-chloro-2-methylphenyl)(4-chlorophenyl)methyl)-2-(4-((3,5-dimethylisoxazol-4-yl)methoxy)phenyl)acetamide;
N-(bis(2-chlorophenyl)methyl)-2-(4-((3,5-dimethylisoxazol-4-yl)methoxy)phenyl)acetamide;
N-(di p-tolylmethyl)-2-(4-((3,5-dimethylisoxazol-4-yl)methoxy)phenyl)acetamide;
2-(4-((3,5-dimethylisoxazol-4-yl)methoxy)phenyl)-N-(1-phenyl-1-(p-tolyl)ethyl)acetamide;
N-((4-chlorophenyl)(2,4-dimethylphenyl)methyl)-2-(4-(2-(3,5-dimethylisoxazol-4-yl)ethoxy)phenyl)acetamide;
N-((4-chlorophenyl)(phenyl)methyl)-2-(4-(((3,5-dimethylisoxazol-4-yl)methyl)(methyl)amino)phenyl)acetamide;
N-((4-chlorophenyl)(phenyl)methyl)-2-(4-(thiazol-4-yl)methoxy)phenyl)acetamide;
N-((4-chlorophenyl)(2,4-dimethylphenyl)methyl)-2-(4-((3-ethyl-5-methylisoxazol-4-yl)methoxy)phenyl)acetamide;
N-((4-chlorophenyl)(phenyl)methyl)-2-(4-((3-(hydroxymethyl)-5-methylisoxazol-4-yl)methoxy)phenyl)acetamide;
N-((4-chlorophenyl)(phenyl)methyl)-2-(4-((5-methylisoxazol-3-yl)methoxy)phenyl)acetamide;
N-((4-chlorophenyl)(phenyl)methyl)-2-(4-((3,5-dimethylisoxazol-4-yl)methoxy)phenyl)-N-methylacetamide;
2-(4-((3,5-dimethylisoxazol-4-yl)methoxy)phenyl)-N-(phenyl(o-tolyl)methyl)acetamide;
N-((4-chlorophenyl)(phenyl)methyl)-2-(4-(oxazol-5-ylmethoxy)phenyl)acetamide;
2-(((3,5-dimethylisoxazol-4-yl)methyl)(4-(2-oxo-2-((phenyl(p-tolyl)methyl)amino)ethyl)phenyl)amino)acetic acid;
N-(bis(4-fluorophenyl)methyl)-2-(4-((3-(hydroxymethyl)-5-methylisoxazol-4-yl)methoxy)phenyl)-2-methylpropanamide;
N-((4-chlorophenyl)(phenyl)methyl)-2-(4-((3,5-dimethylisoxazol-4-yl)methoxy)phenyl)-2-methylpropanamide;
2-(4-((3,5-diisopropylisoxazol-4-yl)methoxy)phenyl)-N-((2,4-dimethylphenyl)(phenyl)methyl)acetamide;
N-((4-((3,5-dimethylisoxazol-4-yl)methoxy)benzyl)-2-(2,4-dimethylphenyl)-N-methyl-2-phenylacetamide;
N-((4-chlorophenyl)(phenyl)methyl)-2-(5-((3,5-dimethylisoxazol-4-yl)methoxy)pyridin-2-yl)acetamide;
N-((4-chlorophenyl)(phenyl)methyl)-2-(4-((3,5-dimethylisoxazol-4-yl)methylthio)phenyl)acetamide;
N-((4-chloro-2-methylphenyl)(phenyl)methyl)-2-(4-(2-(3,5-dimethylisoxazol-4-yl)ethyl)piperazin-1-yl)acetamide;
methyl 2-((3,5-dimethylisoxazol-4-yl)methoxy)-5-(2-(((2,4-dimethylphenyl)(phenyl)methyl)amino)-2-oxoethyl)benzoate;
2-(4-(2-(3,5-dimethylisoxazol-4-yl)-2-hydroxyethoxy)phenyl)-N-((2,4-dimethylphenyl)(phenyl)methyl)acetamide;
2-amino-2-(4-((3,5-dimethylisoxazol-4-yl)methoxy)phenyl)-N-((2,4-dimethylphenyl)(phenyl)methyl)acetamide;
N-((4-chloro-2-methylphenyl)(4-chlorophenyl)methyl)-2-(4-((3,5-dimethylisoxazol-4-yl)methoxy)phenyl)acetamide;
N-((4-chloro-2-methylphenyl)(phenyl)methyl)-2-(4-((3,5-dimethylisoxazol-4-yl)methoxy)phenyl)acetamide;
2-(4-((3,5-dimethylisoxazol-4-yl)methoxy)phenyl)-N-((4-methoxyphenyl)(phenyl)methyl)acetamide;
N-(bis(4-methoxyphenyl)methyl)-2-(4-((3,5-dimethylisoxazol-4-yl)methoxy)phenyl)acetamide;
2-(4-((3,5-dimethylisoxazol-4-yl)methoxy)phenyl)-N((2-methoxyphenyl)(phenyl)methyl)acetamide;
N-((4-(dimethylamino)phenyl) (phenyl)methyl)-2-(4-((3,5-dimethylisoxazol-4-yl) methoxy)phenyl)acetamide;
N-((2-(dimethylamino)phenyl) (phenyl)methyl)-2-(4-((3,5-dimethylisoxazol-4-yl)methoxy)phenyl)acetamide;
2-(4-((3,5-dimethylisoxazol-4-yl)methoxy)phenyl)-N-((4-fluorophenyl)(phenyl)methyl)acetamide;
N-(bis(4-fluorophenyl)methyl)-2-(4-((3,5-dimethylisoxazol-4-yl)methoxy)phenyl)acetamide;
N-((4-chloro-2-methylphenyl)(pyridin-4-yl)methyl)-2-(4-((3,5-dimethylisoxazol-4-yl)methoxy)phenyl)acetamide;
N-((2-chloro-4-methylphenyl)(4-chlorophenyl)methyl)-2-(4-((3,5-dimethylisoxazol-4-yl)methoxy)phenyl)acetamide;
N-((4-chlorophenyl)(o-tolyl)methyl)-2-(4-((3,5-dimethylisoxazol-4-yl)methoxy)phenyl)acetamide;
2-(4-((3,5-dimethylisoxazol-4-yl)methoxy)phenyl)-N-((2,4-dimethylphenyl)(phenyl)methyl)acetamide;
N-((2-chloro-4-methylphenyl)(phenyl)methyl)-2-(4-((3,5-dimethylisoxazol-4-yl)methoxy)phenyl)acetamide;
2-(4-((3,5-dimethylisoxazol-4-yl)methoxy)phenyl)-N-(phenyl(m-tolyl)methyl)acetamide;
2-(4-((3,5-dimethylisoxazol-4-yl)methoxy)phenyl)-N-((4-isopropylphenyl)(phenyl)methyl) acetamide;
N-((4-chloro-2-methylphenyl)(cyclopropyl)methyl)-2-(4-((3,5-dimethylisoxazol-4-yl)methoxy)phenyl)acetamide;
N-(cyclohexyl(2,4-dimethylphenyl)methyl)-2-(4-((3,5-dimethylisoxazol-4-yl)methoxy)phenyl)acetamide;
2-(4-((3,5-dimethylisoxazol-4-yl)methoxy)phenyl)-N-(1-(2,4-dimethylphenyl)-2-methylpropyl)acetamide;
2-(4-((3,5-dimethylisoxazol-4-yl)methoxy)phenyl)-N-(I-(p-tolyl)cyclohexyl)acetamide;
2-(4-((3,5-dimethylisoxazol-4-yl)methoxy)phenyl)-N-(1-phenylcyclohexyl)acetamide;
N-((3,4-dichlorophenyl)(phenyl)methyl)-2-(4-((3,5-dimethylisoxazol-4-yl)methoxy)phenyl)acetamide;
N-((4-chlorophenyl)(2,4-dimethylphenyl)methyl)-2-(4-((3,5-dimethylisoxazol-4-yl)methoxy)phenyl)acetamide;
2-(4-((3,5-dimethyl isoxazol-4-yl)methoxy)phenyl)-N-((2,4-dimethylphenyl)(4-fluorophenyl)methyl)acetamide; 2-(4-((3,5-dimethyloxazol-4-yl)methoxy)phenyl)-N-((2,4-dimethylphenyl)(4-methoxyphenyl)methyl)acetamide;
N-(di-o-tolylmethyl)-2-(4-(2-(3,5-dimethylisoxazol-yl)ethoxy)phenyl)acetamide;
2-4-(2-(3,5-dimethylisoxazol-4-yl)ethoxy)phenyl)-N-((2,6-dimethylphenyl)(phenyl)methyl) acetamide;
2-(4-(2-(3,5-dimethylisoxazol-4-yl)ethoxy)phenyl)-N-((2-isopropylphenyl)(o-tolyl)methyl) acetamide;
2-(4-((3,5-dimethylisoxazol-4-yl)methoxy)phenyl)-N-(I-(3,5-dimethylpyridin-2-yl)-4-methylpentyl)acetamide;
N-((4-chlorophenyl)(2,4-dimethylphenyl)methyl)-2-(4-(2-(3,5-dimethylisoxazol-4-yl)ethoxy)phenyl)acetamide;
N-((4-chlorophenyl)(o-tolyl)methyl)-2-(4-(2-(3,5-dimethylisoxazol-4-yl)ethoxy)phenyl)acetamide;
N-((4-chloro-2-methylphenyl)(phenyl)methyl)-2-(4-(2-(3,5-dimethylisoxazol-4-yl)ethoxy)phenyl)acetamide;
2-(4-(2-(3,5-dimethylisoxazol-4-yl)ethoxy)phenyl)-N-(phenyl(o-tolyl)methyl)acetamide;
N-(di-p-tolylmethyl)-2-(4-(2-(3,5-dimethylisoxazol-4-yl)ethoxy)phenyl)acetamide;
N-((2-chloro-4-methylphenyl)(4-chlorophenyl)methyl)-2-(4-(2-(3,5-dimethylisoxazol-4-yl)ethoxy)phenyl)acetamide;
N-((2-(dimethylamino)phenyl) (phenyl)methyl)-2-(4-(2-(3,5-dimethylisoxazol-4-yl)ethoxy)phenyl)acetamide;
N-((4-chlorophenyl)(phenyl)methyl)-2-(4-((3,5-dimethylisoxazol-4-yl)methoxy)-3-fluorophenyl)acetamide;
N-((4-chlorophenyl)(phenyl)methyl)-2-(4-((3,5-dimethylisoxazol-4-yl)methoxy)-2-fluorophenyl)acetamide;
N-((4-chlorophenyl)(phenyl)methyl)-2-(4-((2-methylthiazol-4-yl)methoxy)phenyl)acetamide;
N-((4-chlorophenyl)(phenyl)methyl)-2-(4-((5-cyclopropylisoxazol-3-yl)methoxy)phenyl)acetamide;
2-(4-((3,5-dimethylisothiazol-4-yl)methoxy)phenyl)-N-(phenyl(p-tolyl)methyl)acetamide;
2-(4-((5-methyl-1,2,4-oxadiazol-3-yl)methoxy)phenyl)-N-(phenyl(p-tolyl)methyl)acetamide;
2-(4-((5-ethyl-1,2,4-oxadiazol-3-yl)methoxy)phenyl)-N-(phenyl(p-tolyl)methyl)acetamide;
2-(4-((3-methyl-1,2,4-oxadiazol-5-yl)methoxy)phenyl)-N-(phenyl(p-tolyl)methyl)acetamide;
2-(4-((5-methyl-1,3,4-oxadiazol-2-yl)methoxy)phenyl)-N-(phenyl(p-tolyl)methyl)acetamide;
2-(4-((3,5-dimethylisoxazol-4-yl)methoxy)phenyl)-N-((4-methoxy-2-methylphenyl)(phenyl)methyl)acetamide;
N-((2-chloro-4-methylphenyl)(phenyl)methyl)-2-(4-((3-(hydroxymethyl)-5-methylisoxazol-4-yl)methoxy)phenyl)acetamide;
N-((2,4-dimethylphenyl)(phenyl)methyl)-2-(4-((3-(hydroxymethyl)-5-methylisoxazol-4-yl)methoxy)phenyl)acetamide;
2-(4-((3,5-dimethylisoxazol-4-yl)methoxy)phenyl)-N-(phenyl(4-(trifluoromethyl)phenyl)methyl)acetamide;
N-((4-chlorophenyl)(phenyl)methyl)-2-(4-((3,5-dimethylisoxazol-4-yl)methoxy)phenyl)propanamide;
N-((4-chlorophenyl)(phenyl)methyl)-2-(4-(2-(3,5-dimethylisoxazol-4-yl)ethoxy)phenyl)acetamide;
2-(4-((3-ethyl-5-methylisoxazol-4-yl)methoxy)phenyl)-N-(phenyl(p-tolyl)methyl)acetamide;
N-(di-p-tolylmethyl)-2-(4-(1-(3,5-dimethylisoxazol-4-yl)ethoxy)phenyl)acetamide;
N-((4-chlorophenyl)(phenyl)methyl)-2-(4-((3-ethyl-5-methylisoxazol-4-yl)methoxy)phenyl)acetamide;
N-((4-chlorophenyl)(phenyl)methyl)-2-(4-((3-methylisoxazol-4-yl)methoxy)phenyl)acetamide;
N-((4-chlorophenyl)(phenyl)methyl)-2-(4-(isoxazol-4-ylmethoxy)phenyl)acetamide;
N-((4-chlorophenyl)(phenyl)methyl)-2-(4-((5-methylthiazol-4-yl)methoxy)phenyl)acetamide;
2-(4-chlorophenyl)-N-(4-((3,5-dimethylisoxazol-4-yl)methoxy)benzyl)-2-phenylacetamide;
2-(1-(3,5-Dimethylisoxazol-4-yl)-2-(4-(2-(((2,4-dimethylphenyl)(phenyl)methyl)amino)-2-oxoethyl)phenoxy)ethoxy)-2-methylpropanoic acid;
(S)—N-((4-chloro-2-methylphenyl)(4-chlorophenyl)methyl)-2-(4-((3,5-dimethylisoxazol-4-yl)methoxy)phenyl)acetamide;
(R)—N-((4-chloro-2-methylphenyl)(4-chlorophenyl)methyl)-2-(4-((3,5-dimethylisoxazol-4-yl)methoxy)phenyl)acetamide;
4-((4-(2-(((4-chloro-2-methylphenyl)(phenyl)methyl)amino)-2-oxoethyl)phenoxy)methyl)-5-methylisoxazole-3-carboxylic acid;
2-((3,5-dimethylisoxazol-4-yl)methoxy)-5-(2-(((2,4-dimethylphenyl)(phenyl)methyl)amino)-2-oxoethyl)benzoic acid;
2-(4-((3,5-dimethylisoxazol-4-yl)methoxy)phenyl)-1-(1-phenyl-3,4-dihydroisoquinolin-2(1H)-yl)ethanone;
N-((4-chloro-2-methylphenyl)(4-chlorophenyl)methyl)-2-(4-(2-(3,5-dimethylisoxazol-4-yl)ethoxy)phenyl)acetamide;
2-(4-(2-(3,5-dimethylisoxazol-4-yl)ethoxy)phenyl)-N-((2,4-dimethylphenyl)(phenyl)methyl)acetamide;
N-(bis(2-chlorophenyl)methyl)-2-(4-((3,5-dimethylisoxazol-4-yl)methoxy)phenyl)acetamide;
2-(4-((3-ethyl-5-methylisoxazol-4-yl)methoxy)phenyl)-N-(phenyl(o-tolyl)methyl)acetamide;

and pharmaceutically acceptable salts thereof.

The meaning of any functional group or substituent thereon at any one occurrence in Formula (I), or any subformula thereof, is independent of its meaning, or any other functional group's or substituent's meaning, at any other occurrence, unless stated otherwise.

The compounds according to Formula (I) may contain one or more asymmetric centers (also referred to as a chiral center) and may, therefore, exist as individual enantiomers, diastereomers, or other stereoisomeric forms, or as mixtures thereof. Chiral centers, such as chiral carbon atoms, may also be present in a substituent such as an alkyl group. Where the stereochemistry of a chiral center present in Formula (I), or in any chemical structure illustrated herein, is not specified the structure is intended to encompass all individual stereoisomers and all mixtures thereof. Thus, compounds according to Formula (I) containing one or more chiral center may be used as racemic mixtures, enantiomerically enriched mixtures, or as enantiomerically pure individual stereoisomers.

Individual stereoisomers of a compound according to Formula (I) which contain one or more asymmetric centers may be resolved by methods known to those skilled in the art. For example, such resolution may be carried out (1) by formation of diastereoisomeric salts, complexes or other derivatives; (2) by selective reaction with a stereoisomer-specific reagent, for example by enzymatic oxidation or reduction, or (3) by gas-liquid or liquid chromatography in a chiral environment, for example, on a chiral support such as silica with a bound chiral ligand or in the presence of a chiral solvent. The skilled artisan will appreciate that where the desired stereoisomer is converted into another chemical entity by one of the separation procedures described above, a further step is required to liberate the desired form. Alternatively, specific stereoisomers may be synthesized by asymmetric synthesis using optically active reagents, substrates, catalysts or solvents, or by converting one enantiomer to the other by asymmetric transformation.

“Enantiomerically enriched” refers to products whose enantiomeric excess is greater than zero. For example, enantiomerically enriched refers to products whose enantiomeric excess is greater than 50% ee, greater than 75% ee, and greater than 90% ee.

“Enantiomeric excess” or “ee” is the excess of one enantiomer over the other expressed as a percentage. As a result, since both enantiomers are present in equal amounts in a racemic mixture, the enantiomeric excess is zero (0% ee). However, if one enantiomer was enriched such that it constitutes 95% of the product, then the enantiomeric excess would be 90% ee (the amount of the enriched enantiomer, 95%, minus the amount of the other enantiomer, 5%).

“Enantiomerically pure” means products whose enantiomeric excess is 99% ee or greater.

When a disclosed compound or its salt is named or depicted by structure, it is to be understood that the compound or salt, including solvates (particularly, hydrates) thereof, may exist in crystalline forms, non-crystalline forms or a mixture thereof. The compound or salt, or solvates (particularly, hydrates) thereof, may also exhibit polymorphism (i.e. the capacity to occur in different crystalline forms). These different crystalline forms are typically known as “polymorphs.” It is to be understood that when named or depicted by structure, the disclosed compound, or solvates (particularly, hydrates) thereof, also include all polymorphs thereof. Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. One of ordinary skill in the art will appreciate that different polymorphs may be produced, for example, by changing or adjusting the conditions used in crystallizing/recrystallizing the compound.

For solvates of the compounds of Formula (I), or salts thereof, that are in crystalline form, the skilled artisan will appreciate that pharmaceutically acceptable solvates may be formed wherein solvent molecules are incorporated into the crystalline lattice during crystallization. Solvates may involve nonaqueous solvents such as ethanol, isopropanol, DMSO, acetic acid, ethanolamine, and ethyl acetate, or they may involve water as the solvent that is incorporated into the crystalline lattice. Solvates wherein water is the solvent that is incorporated into the crystalline lattice are typically referred to as “hydrates.” Hydrates include stoichiometric hydrates as well as compositions containing variable amounts of water. The invention includes all such solvates.

Because of their potential use in medicine, the salts of the compounds of Formula (I) are preferably pharmaceutically acceptable. Suitable pharmaceutically acceptable salts include those described by Berge, Bighley and Monkhouse J. Pharm. Sci (1977) 66, pp 1-19. Salts encompassed within the term “pharmaceutically acceptable salts” refer to non-toxic salts of the compounds of Formula (I).

Salts of the compounds of Formula (I) containing a basic amine or other basic functional group may be prepared by any suitable method known in the art, including treatment of the free base with an inorganic acid, such as hydrochloric acid, hydrobromic acid, sulfuric acid, nitric acid, phosphoric acid, and the like, or with an organic acid, such as acetic acid, trifluoroacetic acid, maleic acid, succinic acid, mandelic acid, fumaric acid, malonic acid, pyruvic acid, oxalic acid, glycolic acid, salicylic acid, pyranosidyl acid, such as glucuronic acid or galacturonic acid, alpha-hydroxy acid, such as citric acid or tartaric acid, amino acid, such as aspartic acid or glutamic acid, aromatic acid, such as benzoic acid or cinnamic acid, sulfonic acid, such as p-toluenesulfonic acid, methanesulfonic acid, ethanesulfonic acid or the like. Examples of pharmaceutically acceptable salts include sulfates, pyrosulfates, bisulfates, sulfites, bisulfites, phosphates, chlorides, bromides, iodides, acetates, propionates, decanoates, caprylates, acrylates, formates, isobutyrates, caproates, heptanoates, propiolates, oxalates, malonates succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, phenylacetates, phenylpropionates, phenylbutrates, citrates, lactates. γ-hydroxybutyrates, glycolates, tartrates mandelates, and sulfonates, such as xylenesulfonates, methanesulfonates, propanesulfonates, naphthalene-1-sulfonates and naphthalene-2-sulfonates.

Salts of the compounds of Formula (I) containing a carboxylic acid or other acidic functional group can be prepared by reacting with a suitable base. Such a pharmaceutically acceptable salt may be made with a base which affords a pharmaceutically acceptable cation, which includes alkali metal salts (especially sodium and potassium), alkaline earth metal salts (especially calcium and magnesium), aluminum salts and ammonium salts, as well as salts made from physiologically acceptable organic bases such as trimethylamine, triethylamine, morpholine, pyridine, piperidine, picoline, dicyclohexylamine, N,N′-dibenzylethylenediamine, 2-hydroxyethylamine, bis-(2-hydroxyethyl)amine, tri-(2-hydroxyethyl)amine, procaine, dibenzylpiperidine, dehydroabietylamine, N,N′-bisdehydroabietylamine, glucamine, N-methylglucamine, collidine, quinine, quinoline, and basic amino acid such as lysine and arginine.

Other non-pharmaceutically acceptable salts, e.g. trifluoroacetate, may be used, for example in the isolation of compounds of the invention, and are included within the scope of this invention.

The invention includes within its scope all possible stoichiometric and non-stoichiometric forms of the salts of the compounds of Formula (I).

If a compound of Formula (I) containing a basic amine or other basic functional group is isolated as a salt, the corresponding free base form of that compound may be prepared by any suitable method known to the art, including treatment of the salt with an inorganic or organic base, suitably an inorganic or organic base having a higher pKₐ than the free base form of the compound. Similarly, if a compound of Formula (I) containing a carboxylic acid or other acidic functional group is isolated as a salt, the corresponding free acid form of that compound may be prepared by any suitable method known to the art, including treatment of the salt with an inorganic or organic acid, suitably an inorganic or organic acid having a lower pKₐ than the free acid form of the compound.

The invention also includes various deuterated forms of the compounds of Formula (I). Each available hydrogen atom attached to a carbon atom may be independently replaced with a deuterium atom. A person of ordinary skill in the art will know how to synthesize deuterated forms of the compounds of Formula (I). Commercially available deuterated starting materials may be employed in the preparation of deuterated forms of the compounds of Formula (I), or they may be synthesized using conventional techniques employing deuterated reagents (e.g. lithium aluminum deuteride or sodium borodeuteride). See for example the compound of Example 5, N-((4-chlorophenyl)(2,4-dimethylphenyl)methyl)-2-(4-((3,5-dimethylisoxazol-4-yl)methoxy)phenyl)acetamide-d₁.

### Methods of Use

Modulators of RORγ can be useful in the treatment of diseases mediated by RORγ, particularly autoimmune or inflammatory diseases and cancer. Such inflammatory or autoimmune diseases include multiple sclerosis, rheumatoid arthritis, psoriasis, Crohn's disease, inflammatory bowel disease, graft-versus-host disease (GVHD), Sjorgen's syndrome, optic neuritis, chronic obstructive pulmonary disease, asthma, type I diabetes, neuromyelitis optica, myasthenia gravis, uveitis, Behcets disease, Guillain-Barre syndrome, psoriatic arthritis, Graves' disease, allergic contact dermatitis, systemic lupus erythematosus, cutaneous lupus erythematosus, ankylosing spondylitis, Hashimoto Thyroiditis, dry eye and glomerulonephritis, myocarditis, especially psoriasis Such cancers include multiple myeloma and lytic bone disease associated with multiple myeloma, acute myelogenous leukemia (AML), head and neck squamous cell carcinoma, bladder carcinoma, gastric cancer, hepatocellular carcinoma, melanoma, medulloblastoma and colon cancer. Accordingly, in another aspect the invention is directed to methods of treating such diseases using a compound of Formula (I) or a pharmaceutically acceptable salt thereof. The methods of treatment of the invention comprise administering an effective amount of a compound according to Formula (I) or a pharmaceutically acceptable salt thereof to a patient (particularly a human) in need thereof.

In a further aspect, the invention is directed to a compound of Formula (I) or a pharmaceutically acceptable salt thereof for use in therapy. In particular, for use in the treatment of diseases mediated by RORγ, particularly autoimmune or inflammatory diseases and cancer, such as those disclosed above.

In a further aspect, the invention is directed to the use of a compound of Formula (I) or a pharmaceutically acceptable salt thereof in the manufacture of a medicament for the treatment of diseases mediated by RORγ, particularly autoimmune or inflammatory diseases and cancer, such as those disclosed above.

As used herein, “treatment” in reference to a condition means: (1) the amelioration or prevention of the condition being treated or one or more of the biological manifestations of the condition being treated, (2) the interference with (a) one or more points in the biological cascade that leads to or is responsible for the condition being treated or (b) one or more of the biological manifestations of the condition being treated, or (3) the alleviation of one or more of the symptoms or effects associated with the condition being treated.

As indicated above. “treatment” of a condition includes prevention of the condition. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof.

An “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein, “patient” refers to a human or a mammal, especially a human.

The compounds of the invention may be administered by any suitable route of administration, including both systemic administration and topical administration. Systemic administration includes oral administration, parenteral administration, transdermal administration, rectal administration, and administration by inhalation. Parenteral administration refers to routes of administration other than enteral, transdermal, or by inhalation, and is typically by injection or infusion. Parenteral administration includes intravenous, intramuscular, and subcutaneous injection or infusion. Inhalation refers to administration into the patient's lungs whether inhaled through the mouth or through the nasal passages. Topical administration includes application to the skin as well as intraocular, otic, intravaginal, and intranasal administration.

The compounds of the invention may be administered once or according to a dosing regimen wherein a number of doses are administered at varying intervals of time for a given period of time. For example, doses may be administered one, two, three, or four times per day. Doses may be administered until the desired therapeutic effect is achieved or indefinitely to maintain the desired therapeutic effect. Suitable dosing regimens for a compound of the invention depend on the pharmacokinetic properties of that compound, such as absorption, distribution, and half-life, which can be determined by the skilled artisan. In addition, suitable dosing regimens, including the amount administered and the duration such regimens are administered, for a compound of the invention depend on the condition being treated, the severity of the condition being treated, the age and physical condition of the patient being treated, the medical history of the patient to be treated, the nature of concurrent therapy, the particular route of administration chosen, the desired therapeutic effect, and like factors within the knowledge and expertise of the skilled artisan. It will be further understood by such skilled artisans that suitable dosing regimens may require adjustment given an individual patient's response to the dosing regimen or over time as individual patient needs change. Typical daily dosages range from 1 mg to 1000 mg.

It will be appreciated by those skilled in the art that certain protected derivatives of compounds of Formula (I), which may be made prior to a final deprotection stage, may not possess pharmacological activity as such, but may, in certain instances, be administered orally or parenterally and thereafter metabolized in the body to form compounds of the invention which are pharmacologically active. Such derivatives may therefore be described as “prodrugs”. Further, certain compounds of the invention may act as prodrugs of other compounds of the invention. All protected derivatives and prodrugs of compounds of the invention are included within the scope of the invention.

Examples of suitable pro-drugs for the compounds of the present invention are described in Drugs of Today, Volume 19, Number 9, 1983, pp 499-538 and in Topics in Chemistry, Chapter 31, pp 306-316 and in “Design of Prodrugs” by H. Bundgaard, Elsevier, 1985, Chapter 1 (the disclosures in which documents are incorporated herein by reference). It will further be appreciated by those skilled in the art, that certain moieties, known to those skilled in the art as “pro-moieties”, for example as described by H. Bundgaard in “Design of Prodrugs” (the disclosure in which document is incorporated herein by reference) may be placed on appropriate functionalities when such functionalities are present within compounds of the invention. Preferred “pro-moieties” for compounds of the invention include: ester, carbonate ester, hemi-ester, phosphate ester, nitro ester, sulfate ester, sulfoxide, amide, carbamate, azo-, phosphamide, glycoside, ether, acetal, and ketal derivatives of the compounds of Formula (I).

Administration of a compound of the invention as a prodrug may enable the skilled artisan to do one or more of the following: (a) modify the onset of the compound in vivo; (b) modify the duration of action of the compound in vivo; (c) modify the transportation or distribution of the compound in vivo; (d) modify the solubility of the compound in vivo; and (e) overcome or overcome a side effect or other difficulty encountered with the compound.

The invention further includes the use of compounds of the invention as an active therapeutic substance, in particular in the treatment of diseases mediated by RORγ. In another embodiment, the invention relates to the use of compounds of the invention in the preparation of a medicament for the treatment of diseases mediated by RORγ.

Examples of such diseases include autoimmune or inflammatory diseases such as multiple sclerosis, rheumatoid arthritis, psoriasis, Crohn's disease, inflammatory bowel disease, Sjorgen's syndrome, optic neuritis, chronic obstructive pulmonary disease, asthma, type I diabetes, neuromyelitis optica, Myasthenia Gravis, uveitis, Guillain-Barre syndrome, psoriatic arthritis, Graves' disease, allergic contact dermatitis, systemic lupus erythematosus, cutaneous lupus erythematosus, ankylosing spondylitis, Hashimoto Thyroiditis, Dry Eye, glomerulonephritis, myocarditis and cancer diseases including multiple myeloma and lytic bone disease associated with multiple myeloma, acute myclogenous leukemia (AML), head and neck squamous cell carcinoma, bladder carcinoma, gastric cancer, hepatocellular carcinoma, melanoma, medulloblastoma and colon cancer.

The invention includes the use of compounds of the invention for the preparation of a composition for treating or ameliorating diseases mediated by RORγ in a subject in need thereof, wherein the composition comprises a mixture of one or more of the compounds of the invention and an optional pharmaceutically acceptable excipient.

The compounds of the invention may be used alone or in combination with one or more other therapeutic agents. Accordingly the present invention provides a combination comprising a compound of Formula (I) or a pharmaceutically acceptable salt thereof and one or more other therapeutic agents. Such combinations may be presented individually (wherein each active is in separate composition) or the actives are presented in a combined composition.

This invention provides a combination of a compound of Formula (I), or a pharmaceutically acceptable salt thereof, and one or more therapeutic agents for the treatment of an inflammatory disease and/or an autoimmune disease, for example, a TNF-a inhibitor; a non-selective COX-1/COX-2 inhibitor; a selective COX-2 inhibitor, such as celecoxib; agents including methotrexate, leflunomide, sulfasalazine, azathioprine, penicillamine, bucillamine, actarit, mizoribine, lobenzarit, hydroxychloroquine, d-penicillamine, aurothiomalate, auranofin, parenteral and/or oral gold, cyclophosphamide, a BAFF/APRIL inhibitor. CTLA-4-Ig, or a mimetic of CTLA-4-Ig; 5-lipoxygenase (5-LO) inhibitor, or a 5-lipoxygenase activating protein (FLAP) antagonist; a leukotriene modifier, including a leukotriene receptor antagonist, such as montelukast, zafirlukast, pranlukast; a phosphodiesterase type IV (PDE-IV) inhibitor, such as cilomilast (ariflo) or roflumilast; an antihistamine H1 receptor antagonist; anticholinergic agents such as muscarinic antagonists (ipratropium bromide and tiotropium bromide), as well as selective muscarinic M3 antagonists; β-adrenoceptor agonists such as salmeterol, formoterol, arformoterol, terbutaline, metaproterenol, albuterol and the like; a DP receptor antagonist, such as S-5751 and laropiprant; TP receptor antagonists such as seratrodast; neurokinin antagonists (1 NK2); VLA-4 antagonists; a corticosteroid, such as triamcinolone acetonide, budesonide, beclomethasone, fluticasone and mometasone; insulin-like growth factor type I (IGF-1) mimetic; kinase inhibitors including Janus Kinase inhibitors (e.g., JAK 1 and/or JAK2 and/or JAK 3 and/or TYK2), p38 MAPK, Syk or IKK2; rituximab; selective co-stimulation modulator such as abatacept; IL-1 inhibitor anakinra, IL-6 inhibitor tocilizumab, and IL 12/IL-23 inhibitor ustekimumab; anti-IL17 antibody, anti-IL17R antibody, anti-IL21 antibody, or anti-IL22 antibody, S1P1 agonists including fingolimod; interferon beta 1; natalizumab; a mTOR inhibitor such as rapamycin, cyclosporine, tacrolimus; non-steroidal antiinflammatory agent (NSAID), including alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid, and tioxaprofen, indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin, and zomepirac, flufenamic acid, meclofenamic acid, mefenamic acid, niflumic acid, tolfenamic acid, diflunisal and flufenisal, isoxicam, piroxicam, sudoxicam, tenoxican, acetyl salicylic acid, apazone, bezpiperylon, feprazone, mofebutazone, oxyphenbutazone, phenylbutazone; fumaric acid derivative, BG-12; chemokine or chemokine receptor inhibitor, such as a CCR-1, CCR-2, CCR-3 and CCR-9 antagonist.

This invention further provides a combination of a compound of Formula (I), or a pharmaceutically acceptable salt thereof, and one or more therapeutic agents for the treatment of multiple myeloma, for example. Bortezomib-dexamethasone. Bortezomib-dexamethasone-cyclophosphamide, Bortezomib-dexamethasone-lenalidomide, Lenalidomide-dexamethasone, Melphalan-prednisone-thalidomide, Melphalan-prednisone-bortezomib, Melphalan-prednisone-lenalidomide, Lenalidomide-dexamethasone- clarithromycin and any of the above combinations plus agents used to treat bone disease in multiple myeloma including bisphosponates, RANK-L inhibitors such as Denusomab and anabolic bone building drugs such as parathyroid hormone (PTH).

This invention also provides a combination of a compound of Formula (I), or a pharmaceutically acceptable salt thereof, and one or more therapeutic agents for the treatment of colon and/or rectal cancer, for example FOLFOX® (leucovorin [folinic acid], 5-Fluoruracil, and oxaliplatin), FOLFIRI®) (leucovorin, 5-Fluoruracil, and irinotecan), CapeOX® (capecitabine and oxaliplatin), any of the above combinations plus either bevacizumab or cetuximab (but not both), 5-Fluoruracil and leucovorin, with or without bevacizumab. Capecitabine, with or without bevacizumab, FOLFOXIRI® (leucovorin, 5-Fluoruracil, oxaliplatin, and irinotecan). Irinotecan, with or without cetuximab, Cetuximab alone, and Panitumumab alone.

### Compositions

The compounds of the invention will normally, but not necessarily, be formulated into pharmaceutical compositions prior to administration to a patient. Accordingly, in another aspect the invention is directed to pharmaceutical compositions comprising a compound of the invention and one or more pharmaceutically acceptable excipient(s).

The pharmaceutical compositions of the invention may be prepared and packaged in bulk form wherein an effective amount of a compound of the invention can be extracted and then given to the patient such as with powders, syrups, and solutions for injection. Alternatively, the pharmaceutical compositions of the invention may be prepared and packaged in unit dosage form. For oral application, for example, one or more tablets or capsules may be administered. A dose of the pharmaceutical composition contains at least a therapeutically effective amount of a compound of this invention (i.e., a compound of Formula I or a salt, particularly a pharmaceutically acceptable salt, thereof). When prepared in unit dosage form, the pharmaceutical compositions may contain from 1 mg to 1000 mg of a compound of this invention.

The pharmaceutical compositions of the invention typically contain one compound of the invention. However, in certain embodiments, the pharmaceutical compositions of the invention contain more than one compound of the invention. For example, in certain embodiments the pharmaceutical compositions of the invention contain two compounds of the invention. In addition, the pharmaceutical compositions of the invention may optionally further comprise one or more additional therapeutically active compounds.

As used herein, “pharmaceutically acceptable excipient” means a pharmaceutically acceptable material, composition, or vehicle involved in giving form or consistency to the pharmaceutical composition. Each excipient must be compatible with the other ingredients of the pharmaceutical composition when commingled such that interactions which would substantially reduce the efficacy of the compound of the invention when administered to a patient and interactions which would result in pharmaceutical compositions that are not pharmaceutically acceptable are avoided. In addition, each excipient must of course be of sufficiently high purity to render it pharmaceutically acceptable.

The compounds of the invention and the pharmaceutically acceptable excipient or excipients will typically be formulated into a dosage form adapted for administration to the patient by the desired route of administration. For example, dosage forms include those adapted for (1) oral administration such as tablets, capsules, caplets, pills, troches, powders, syrups, elixers, suspensions, solutions, emulsions, sachets, and cachets; (2) parenteral administration such as sterile solutions, suspensions, and powders for reconstitution; (3) transdermal administration such as transdermal patches; (4) rectal administration such as suppositories; (5) inhalation such as dry powders, aerosols, suspensions, and solutions; and (6) topical administration such as creams, ointments, lotions, solutions, pastes, sprays, foams, and gels.

Suitable pharmaceutically acceptable excipients will vary depending upon the particular dosage form chosen. In addition, suitable pharmaceutically acceptable excipients may be chosen for a particular function that they may serve in the composition. For example, certain pharmaceutically acceptable excipients may be chosen for their ability to facilitate the production of uniform dosage forms. Certain pharmaceutically acceptable excipients may be chosen for their ability to facilitate the production of stable dosage forms. Certain pharmaceutically acceptable excipients may be chosen for their ability to facilitate the carrying or transporting of the compound or compounds of the invention once administered to the patient from one organ, or portion of the body, to another organ, or portion of the body. Certain pharmaceutically acceptable excipients may be chosen for their ability to enhance patient compliance.

Suitable pharmaceutically acceptable excipients include the following types of excipients: diluents, fillers, binders, disintegrants, lubricants, glidants, granulating agents, coating agents, wetting agents, solvents, co-solvents, suspending agents, emulsifiers, sweeteners, flavoring agents, flavor masking agents, coloring agents, anticaking agents, hemectants, chelating agents, plasticizers, viscosity increasing agents, antioxidants, preservatives, stabilizers, surfactants, and buffering agents. The skilled artisan will appreciate that certain pharmaceutically acceptable excipients may serve more than one function and may serve alternative functions depending on how much of the excipient is present in the formulation and what other ingredients are present in the formulation.

Skilled artisans possess the knowledge and skill in the art to enable them to select suitable pharmaceutically acceptable excipients in appropriate amounts for use in the invention. In addition, there are a number of resources that are available to the skilled artisan which describe pharmaceutically acceptable excipients and may be useful in selecting suitable pharmaceutically acceptable excipients. Examples include Remington's Pharmaceutical Sciences (Mack Publishing Company, The Handbook of Pharmaceutical Additives (Gower Publishing Limited), and The Handbook of Pharmaceutical Excipients (the American Pharmaceutical Association and the Pharmaceutical Press).

The pharmaceutical compositions of the invention are prepared using techniques and methods known to those skilled in the art. Some of the methods commonly used in the art are described in Remington's Pharmaceutical Sciences (Mack Publishing Company).

In one aspect, the invention is directed to a solid oral dosage form such as a tablet or capsule comprising a safe and effective amount of a compound of the invention and a diluent or filler. Suitable diluents and fillers include lactose, sucrose, dextrose, mannitol, sorbitol, starch (e.g. corn starch, potato starch, and pre-gelatinized starch), cellulose and its derivatives (e.g. microcrystalline cellulose), calcium sulfate, and dibasic calcium phosphate. The oral solid dosage form may further comprise a binder. Suitable binders include starch (e.g. corn starch, potato starch, and pre-gelatinized starch), gelatin, acacia, sodium alginate, alginic acid, tragacanth, guar gum, povidone, and cellulose and its derivatives (e.g. microcrystalline cellulose). The oral solid dosage form may further comprise a disintegrant. Suitable disintegrants include crospovidone, sodium starch glycolate, croscarmelose, alginic acid, and sodium carboxymethyl cellulose. The oral solid dosage form may further comprise a lubricant. Suitable lubricants include stearic acid, magnesium stearate, calcium stearate, and talc.

### Compound Preparation

The compounds of Formula (I) may be obtained by using synthetic procedures illustrated in the Schemes below or by drawing on the knowledge of a skilled organic chemist. The reaction sequences provided in these Schemes are applicable for producing compounds of the invention having a variety of different X¹-X⁵, R¹, R³, R^{3a}, R⁴, R^{4a}, K¹-K⁴, and A¹-A⁴ groups, as defined above, employing appropriate precursors. The skilled artisan will appreciate that if a substituent described herein is not compatible with the synthetic methods described herein, the substituent may be protected with a suitable protecting group that is stable to the reaction conditions. The protecting group may be removed at a suitable point in the reaction sequence to provide a desired intermediate or target compound. Suitable protecting groups and the methods for protecting and de-protecting different substituents using such suitable protecting groups are well known to those skilled in the art; examples of which may be found in T. Greene and P. Wuts, Protecting Groups in Chemical Synthesis (3rd ed.), John Wiley & Sons, NY (1999). In some instances, a substituent may be specifically selected to be reactive under the reaction conditions used. Under these circumstances, the reaction conditions convert the selected substituent into another substituent that is either useful as an intermediate compound or is a desired substituent in a target compound.

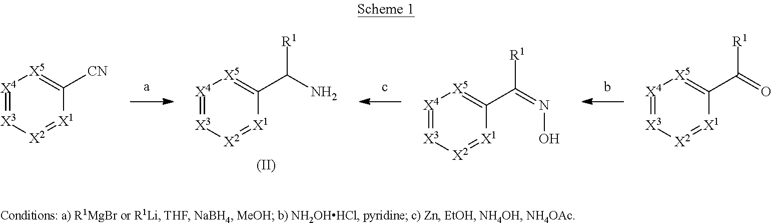

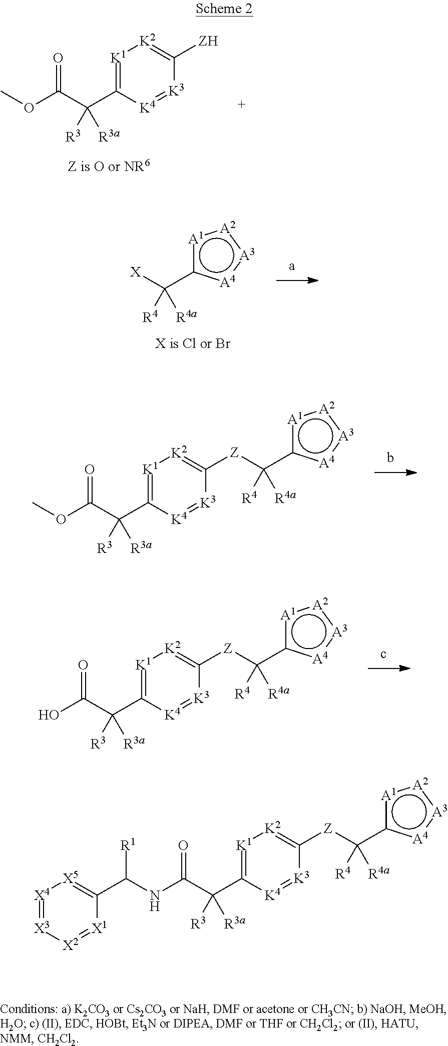

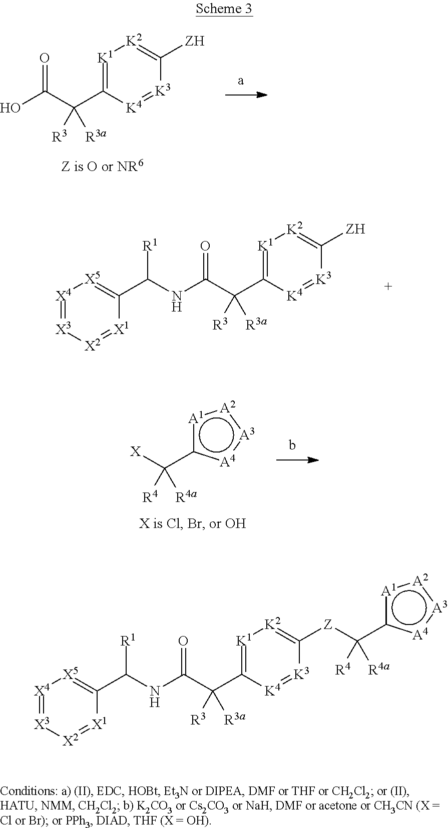

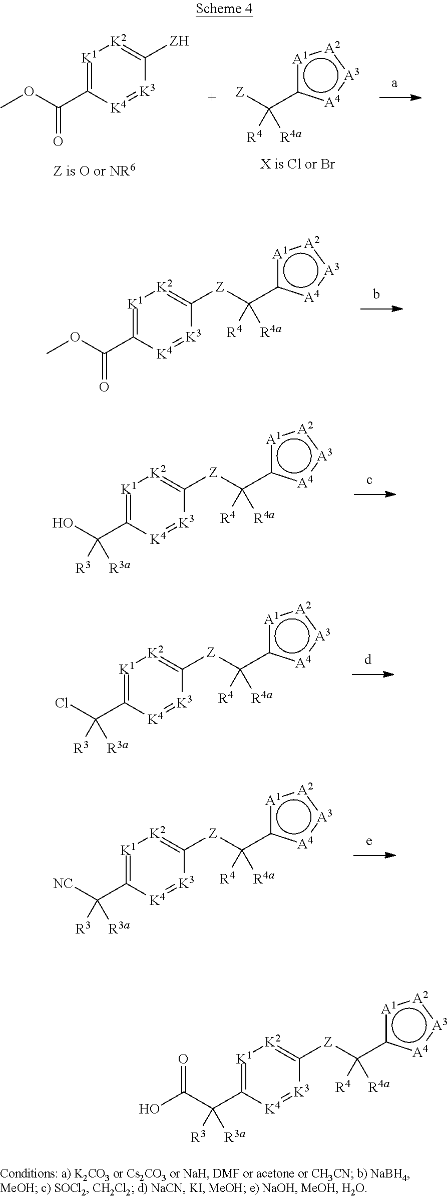

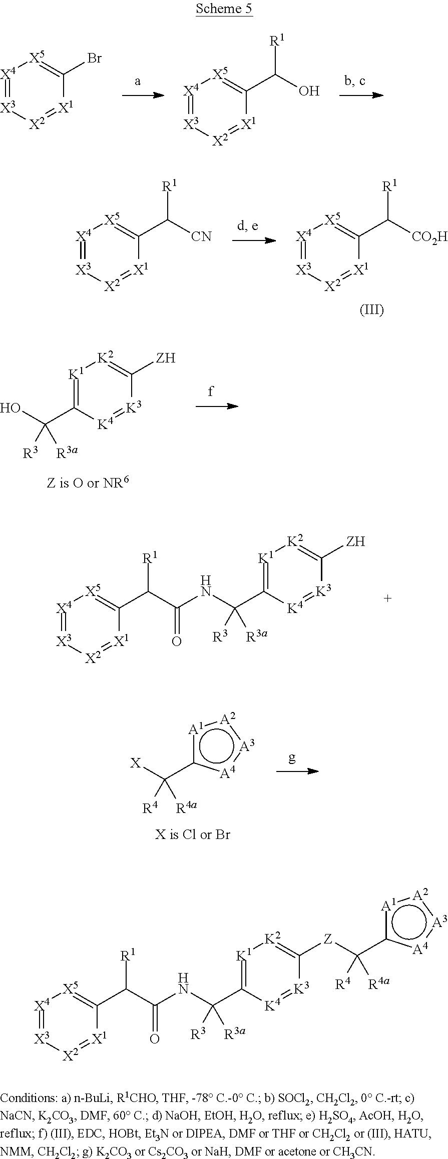

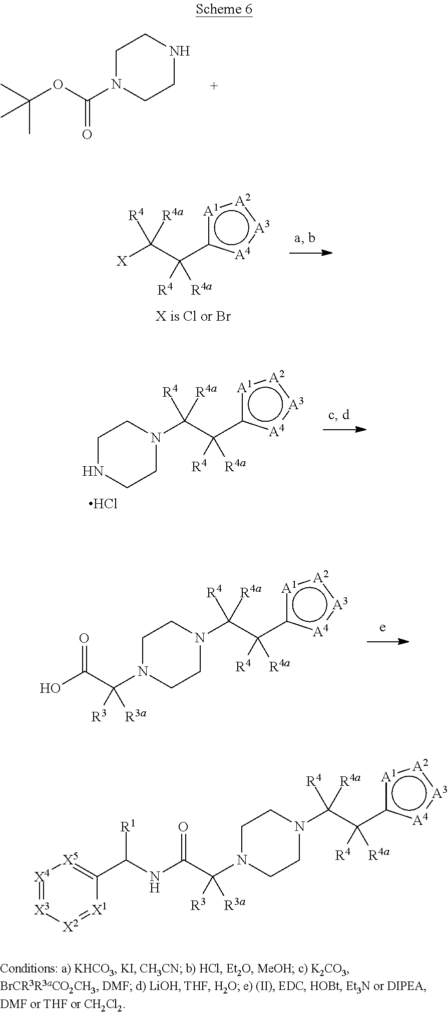

### EXAMPLES

The following examples illustrate the invention. These examples are not intended to limit the scope of the present invention, but rather to provide guidance to the skilled artisan to prepare and use the compounds, compositions, and methods of the present invention. While particular embodiments of the present invention are described, the skilled artisan will appreciate that various changes and modifications can be made without departing from the spirit and scope of the invention.

Compounds names were generated using the software program ChemBioDraw Ultra V12.0 available from CambridgeSoft Corporation, 100 CambridgePark Drive, Cambridge, Mass. 02140 USA (http://www.cambridgesoft.com).

### ABBREVIATIONS

AcOH acetic acid
AIBN azobisisobutyronitrile
AlCl₃ aluminum trichloride
aq. aqueous
Ar argon gas
Br₂ bromine
CBr₄ carbon tetrabromide
CCl₄ carbon tetrachloride
CH₂Cl₂ dichloromethane
CH₃CN acetonitrile
CH₃I methyl iodide
(CH₂O)ₙ paraformaldehyde
CH₃SO₃H methanesulfonic acid
conc. concentrated
Cs₂CO₃ cesium carbonate
CuBr copper(I) bromide
CuCN copper(I) cyanide
CuI copper(I) iodide
(COCl)₂ oxalyl chloride
DIPEA N,N-diisopropylethylamine
DMAP 4-(dimethylamino)pyridine
DME 1,2-dimethoxyethane
DMF N,N-dimethylformamide
DMSO dimethylsulfoxide
EtOAc ethyl acetate
EDC N-(3-dimethylaminopropyl)-N′-ethylcarbodiimide hydrochloride
Et₃N triethylamine
Et₂O diethyl ether
EtOH ethanol
FeSO₄ iron(II) sulfate
h hour(s)
H₂ hydrogen gas
HATU O-(7-azabenzotriazol-1-yl)-N,N,N′,N′-tetramethyluronium hexafluorophosphate
HBr hydrobromic acid
HCl hydrochloric acid
H₂O water
HNO₃ nitric acid
HOBt hydroxybenzotriazole
HPLC high-performance liquid chromatography
H₂SO₄ sulfuric acid
I₂ iodine
i-PrMgCl isopropylmagnesium chloride
K₂CO₃ potassium carbonate
K₃Fe(CN)₆ potassium ferricyanide
KOt-Bu potassium tert-butoxide
K₃PO₄ potassium phosphate tribasic
LCMS liquid chromatography mass spectrometry
LiAlH₄ lithium aluminum hydride
LiOH lithium hydroxide
m-CPBA meta-chloroperbenzoic acid
MeMgBr methyl magnesium bromide
MeOH methanol
Mg magnesium
MgCl₂ magnesium chloride
min minute(s)
MnO₂ manganese dioxide
N₂ nitrogen gas
NaBH₄ sodium borohydride
NaCN sodium cyanide
Na₂CO₃ sodium carbonate
NaH sodium hydride
NaHCO₃ sodium bicarbonate
NaHSO₃ sodium bisulfite
NaN₃ sodium azide
NaOH sodium hydroxide
Na₂SO₄ sodium sulfate
n-BuLi n-butyllithium
NH₄Cl ammonium chloride
NMM N-methylmorpholine
PCC pyridinium chlorochromate
Pd/C palladium on carbon
Pd(dppf)Cl₂ [1,1′-bis(diphenylphosphino)ferrocene]dichloropalladium(II)
Pd(PPh₃)₄ tetrakis(triphenylphosphine)palladium(0)
PhNO₂ nitrobenzene
POCl₃ phosphoryl chloride
PPh₃ triphenylphosphine
p-TsOH para-toluene sulfonic acid
R_{f} retention factor
rt room temperature
Rₜ retention time
SOCl₂ thionyl chloride
TFA trifluoroacetic acid
THF tetrahydrofuran
TLC thin layer chromatography
®T3P 2,4,6-tripropyl-1,3,5,2,4,6-trioxatriphosphorinane 2,4,6-trioxide
Zn zinc powder

### LCMS Conditions

LCMS-P1: Column: Waters Sunfire C18, 3.5 μm, 50×4.6 mm; Temperature: 50° C.; Mobile Phase: A: water (0.05% TFA) B: acetonitrile (0.05% TFA); Gradient: 5% B for 0.2 min, increase to 95% B within 1.2 min. 95% B for 1.6 min, return to 5% B within 0.01 min.; Flow Rate: 1.8 mL/min; Detection: PDA 190-400 nm

LCMS-G7: Column: XBridge C18. 3.6 μm, 50×4.6 mm; Temperature: 50° C.; Mobile Phase: A: water (0.1% formic acid) B: methanol; Gradient: 10% B for 0.1 min, increase to 95% B within 2.5 min, 95% B for 2.5 min, return to 10% B within 0.1 min, 10% B for 2 min.; Flow Rate: 1.0 mL/min; Detection: PDA 190-400 nm

LCMS-G9: Column: XBridge C18, 3.6 μm, 50×4.6 mm; Temperature: 50° C.; Mobile Phase: A: water (0.1% ammonium acetate) B: methanol; Gradient: 10% B for 0.2 min, increase to 95% B within 5 min, 95% B for 2 min, return to 10% B within 0.1 min, 10% B for 2 min.; Flow Rate: 0.8 mL/min; Detection: PDA 190-400 nm

LCMS-G12: Column: Sunfire C18. 5 μm, 50×4.6 mm; Temperature: 50° C.; Mobile Phase: A: water (0.1% formic acid) B: methanol; Gradient: 30% B for 0.1 min, increase to 90% B within 4 min, 99% B for 4 min, return to 30% B within 0.1 min. 10% B for 2 min.; Flow Rate: 0.8 mL/min; Detection: PDA 190-400 nm

LCMS-G30: Column: Eclipse XDB C18. 5 μm, 250×4.6 mm; Temperature: 50° C.; Mobile Phase: A: water (0.05% TFA) B: acetonitrile (0.05% TFA); Gradient: 30% B for 0.2 min, increase to 95% B within 15 min, 95% B for 5 min, return to 30% B within 3 min 30% B for 5 min.; Flow Rate: 0.8 mL/min; Detection: PDA 190-400 nm

LCMS-X: Column: Eclipse XDB C18, 5 μm, 150×4.6 mm; Temperature: 50° C.; Mobile Phase: A: water (0.1% formic acid) B: acetonitrile (0.1% formic acid); Gradient: 10% B for 0.1 min, increase to 90% B within 5 min, 100% within 2 min, 100% B for 4 min, return to 10% B within 0.01 min, 10% B for 1 min.; Flow Rate: 1.0 mL/min; Detection: PDA 190-400 nm

LCMS-T1: Column: Eclipse XDB C18, 5 μm, 150×4.6 mm; Temperature: 50° C.; Mobile Phase: water (0.05% TFA) B: acetonitrile (0.05% TFA); Gradient: 5% B for 0.1 min, increase to 95% B within 7 min, 100% within 2 min, return to 5% B within 0.1 min, 5% B for 3 min.; Flow Rate: 1.0 mL/min; Detection: PDA 190-400 nm

### Example 1

### N-((4-chloro-2-methy phenyl)(p-tolyl)methyl)-2-(4-((3,5-dimethylisoxazol-4-yl)methoxy)phenyl) acetamide

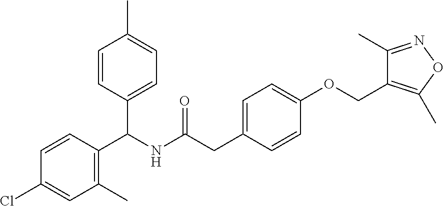 [SMILES: Cc1ccc(C(NC(=O)Cc2ccc(OCc3c(C)noc3C)cc2)c2ccc(Cl)cc2C)cc1]

### (a) methyl 2-(4-hydroxyphenyl)acetate

To a solution of 2-(4-hydroxyphenyl)acetic acid (5.0 g, 32.89 mmol) in methanol (75 mL), HCl gas was purged for 2 hours at 0° C. After completion of the reaction, the reaction mixture was cooled to rt and methanol was distilled out under reduced pressure. The crude obtained was dissolved in water and neutralized to pH=7 using sodium bi carbonate solution. The aqueous layer was extracted with ethyl acetate (2×50 mL). The combined organic layers were dried over Na₂SO₄ and distilled under reduced pressure to provide the title compound. LCMS-P1: 167.2 [M+H]⁺; Rₜ: 1.276 min.

### (b) methyl 2-(4-((3,5-dimethylisoxazol-4-yl)methoxy)phenyl)acetate

To a solution of methyl 2-(4-hydroxyphenyl)acetate (3.0 g, 18.0 mmol) in dimethylformamide (35 mL) was added potassium carbonate (3.7 g, 27.1 mmol), and the reaction mixture was stirred at rt for 30 min. 4-(chloromethyl)-3,5-dimethylisoxazole (2.62 g, 21.6 mmol) was then added and the resulting mixture was stirred at 80° C. for 6 h. After completion of the reaction, water (30 mL) was added and the reaction mixture was extracted with ethyl acetate (2×50 mL). The organic layer was dried over Na₂SO₄ and concentrated to obtain a crude product which was purified by silica gel column chromatography using (30% EtOAc/hexanes) to provide the title compound (3.3 g, 67%). ¹H NMR (400 MHz, DMSO-d₆) δ ppm 7.17-7.20 (d, 2H), 6.94-6.96 (d, 2H), 4.88 (s, 2H), 3.60 (s, 3H), 3.41 (s, 2H), 2.39 (s, 3H), 2.20 (s, 3H). MS (ESI+)=276.12 (M+H).

### (c) 2-(4-((3,5-dimethylisoxazol-4-yl)methoxy)phenyl)acetic acid

To a solution of methyl 2-(4-((3,5-dimethylisoxazol-4-yl)methoxy)phenyl)acetate (3.2 g, 11.6 mmol) in methanol (35 mL), sodium hydroxide (0.93 g, 23.2 mmol) in 50 mL of water was added dropwise and the reaction mixture was stirred at rt for 2 h. After completion of the reaction, the methanol was distilled off at reduced pressure and water (50 mL) was added to the reaction mixture. The aqueous layer was washed with ethyl acetate and discarded. Then, the aqueous layer was acidified to pH=3 using 10% Hydrochloric acid and extracted with ethyl acetate (2×50 mL). The organic layer was dried over Na₂SO₄ and concentrated to provide the title compound (2.9 g, 95.70%). ¹H NMR (400 MHz, DMSO-d₆) δ ppm 7.03-7.16 (d, 2H), 6.91-6.93 (d, 2H), 4.84 (s, 2H), 3.46 (s, 2H), 2.35 (s, 3H), 2.17 (s, 3H). MS (ESI+) 262.06 (M+H).

### (d) p-tolylmagnesium bromide

Magnesium turnings (0.224 g, 9.35 mmol) were added to a three neck RBF equipped with nitrogen flow and cold water condenser. Anhydrous THF (25 mL) was added to the reaction mixture and heated at 70-80° C. A solution of 1-bromo-4-methylbenzene (0.80 g, 4.67 mmol) in THF (10 mL) was added over 20 minutes dropwise to the reaction mixture and the resulting mixture was refluxed at 80° C. for 2 h. The resulting gray mixture was cooled to 10° C. and used in next step without further purification.

### (e) (4-chloro-2-methylphenyl)(p-tolyl)methanamine

To a solution of 4-chloro-2-methylbenzonitrile (0.50 g, 3.31 mmol) in anhydrous THF (15 mL) at 0° C. was added solution of p-tolylmagnesium bromide in THF (15 mL) slowly over 10 minutes and the resulting mixture was allowed to warm to rt. The reaction mixture was stirred at rt for 5 h and heated to 60° C. where it was further stirred for 2 h. After completion of the imine formation, the reaction mixture was cooled to 0° C. and 5 mL of methanol was added very slowly followed by sodium borohydride (0.244 g, 6.62 mmol). The resulting mixture was warmed to rt and stirred overnight. After completion of the reaction, water (10 mL) was added in to the reaction mixture and the mixture was extracted with ethyl acetate (2×40 mL). The combined organic layers were washed with brine (25 mL), dried over Na₂SO₄, and evaporated to obtained a crude product which was purified using silica gel column chromatography using 10% ethyl acetate in hexanes to obtain the title compound (0.40 g, 40.0%) as a light brown oil. ¹H NMR (400 MHz, DMSO-d₆) δ ppm 7.54-7.56 (d, 1H), 7.22-7.28 (d, 1H), 7.15-7.21 (m, 3H), 7.11-7.13 (m, 2H), 5.16 (s, 1H), 2.24 (s, 3H), 2.21 (s, 3H).

### (f) N-((4-chloro-2-methylphenyl)(p-tolyl)methyl)-2-(4-((3,5-dimethylisoxazol-4-yl)methoxy)phenyl) acetamide

To a solution of 2-(4-((3,5-dimethylisoxazol-4-yl)methoxy)phenyl)acetic acid (0.20 g 0.766 mmol) in tetrahydrofuran (25 mL), EDC (0.20 g, 1.149 mmol) was added portion wise and the reaction was stirred at rt for 2 h. (4-chloro-2-methylphenyl)(p-tolyl)methanamine (0.206 g, 0.842 mmol), HOBt (0.117 g, 0.766 mmol), and triethylamine (0.318 mL, 2.29 mmol) were added and the reaction mixture was stirred for 24 h at rt. After completion of the reaction, water (50 mL) was added to the reaction mixture and the mixture was extracted with ethyl acetate (2×50 mL). The organic layer was dried over Na₂SO₄ and concentrated to obtain a crude product which was purified by silica gel column chromatography using 30% EtOAc/hexane as mobile phase to provide the title compound (0.115 g, 30.74%). LCMS-X1: 489.3 [M+H]⁺; Rₜ=5.89 min. ¹H NMR (400 MHz, DMSO-d₆) δ ppm 8.85-8.87 (d, 1H), 7.12-7.29 (m, 6H), 7.01-7.09 (m, 2H), 6.91-6.99 (d, 2H), 6.11-6.13 (d, 1H), 4.88 (s, 2H), 3.43 (s, 2H), 2.38 (s, 3H), 2.27 (s, 3H), 2.20 (s, 3H), 2.17 (s, 3H).

### Example 2

### N-((4-chloro-2-methylphenyl)(4-chlorophenyl)methyl)-2-(4-((3,5-dimethylisoxazol-4-yl)methoxy)phenyl)acetamide

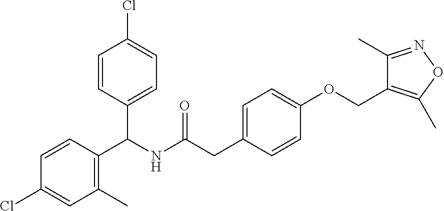 [SMILES: Cc1cc(Cl)ccc1C(NC(=O)Cc1ccc(OCc2c(C)noc2C)cc1)c1ccc(Cl)cc1]

### (a) (4-chloro-2-methylphenyl)(4-chlorophenyl)methanamine

This compound was synthesized from 4-chloro-2-methylbenzonitrile and (4-chlorophenyl)magnesium bromide essentially as described in example 1 (e) to give the title compound (0.311 g, 35.42%) out of which 224 mg was used in the next step without further purification. ¹H NMR (400 MHz, DMSO-d₆) δ ppm 7.49-7.51 (d, 1H), 7.37-7.40 (d, 2H), 7.26-7.35 (m, 3H), 7.21-7.22 (d, 1H), 5.29 (s, 1H), 2.08 (s, 3H).

### (b) N-((4-chloro-2-methylphenyl)(4-chlorophenyl)methyl)-2-(4-((3,5-dimethylisoxazol-4-yl)methoxy)phenyl)acetamide

This compound was synthesized from 2-(4-((3,5-dimethylisoxazol-4-yl)methoxy)phenyl)acetic acid and (4-chloro-2-methylphenyl)(4-chlorophenyl)methanamine essentially as described in example 1 (f), except the title compound was isolated as follows: after completion of the reaction, water (10 mL) was added into the reaction mixture very slowly with cooling and the obtained white solid was filtered off and washed with water (20 mL) and hexanes (20 mL) and dried under reduced pressure to obtain the title compound (240 mg, 55.98%). LCMS-X1: 509.2 [M+H]; Rₜ=6.03 min. ¹H NMR (400 MHz, DMSO-d₆) δ ppm 8.92-8.94 (d, 1H), 7.39-7.41 (d, 2H), 7.28 (s, 1H), 7.17-7.25 (m, 5H), 7.04-7.06 (d, 1H), 6.92-6.94 (d, 2H), 6.17-6.19 (d, 1H), 4.88 (s, 2H), 3.45 (s, 2H), 2.39 (s, 3H), 2.20 (s, 6H).

### Example 3

### N-(bis(2-chlorophenyl)methyl)-2-(4-((3,5-dimethylisoxazol-4-yl)methoxy)phenyl)acetamide

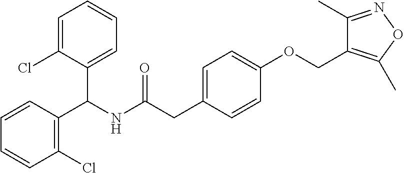 [SMILES: Cc1noc(C)c1COc1ccc(CC(=O)NC(c2ccccc2Cl)c2ccccc2Cl)cc1]

### (a) bis(2-chlorophenyl)methanone oxime

To a solution of bis(2-chlorophenyl)methanone (1.0 g, 3.98 mmol) in pyridine (10.0 mL) was added hydroxylamine hydrochloride (1.10 g, 15.90 mmol), and the reaction was heated at reflux temperature overnight. After completion of the reaction, the reaction mixture was concentrated, diluted with EtOAc (50 mL), and washed with water (25 mL) and 2M HCl solution. The combined organic layers were dried over Na₂SO₄ and concentrated. The title compound was purified by triturating with diethyl ether (10 mL) to provide (0.80 g, 75.54%). ¹H NMR (400 MHz, DMSO-d₆) δ ppm 11.84 (s, 1H), 7.44-7.51, (m, 3H), 7.40-7.44 (m, 2H), 7.36-7.40 (m, 2H), 7.27-7.29 (dd 1H).

### (b) bis(2-chlorophenyl)methanamine

To a solution of bis(2-chlorophenyl)methanone oxime (0.800 g, 3.00 mmol) in ethanol (4.0 mL) was added concentrated ammonia solution (20 mL), ammonium acetate (0.115 g, 1.50 mmol), and zinc powder (1.05 g, 16.23 mmol). The mixture was heated at reflux temperature for 4 h. After completion of the reaction, the reaction mixture was cooled to rt and filtered through Celite. A 10% NaOH solution was added and then the mixture was washed with ethyl acetate (100 mL). The organic layer was dried over Na₂SO₄ and concentrated. Title compound was obtained (0.80 g, 75.54%) and was used in the next step without any further purification. ¹H NMR (400 MHz, DMSO-d₆) δ ppm 7.38-7.41, (m, 4H), 7.32-7.35 (m, 2H), 7.25-7.30 (m, 2H), 5.66 (s 1H), 2.33 (br, 2H).

### (c) N-(bis(2-chlorophenyl)methyl)-2-(4-((3,5-dimethylisoxazol-4-yl)methoxy)phenyl)acetamide

This compound was synthesized from bis(2-chlorophenyl)methanamine and 2-(4-((3,5-dimethylisoxazol-4-yl) methoxy)phenyl) acetic acid essentially as described in example 1 (f) except the reaction was carried out in DMF. (87 mg, 22.19%). LCMS-X: 495.2 [M+H]⁺; Rₜ=6.81 min. ¹H NMR (400 MHz, DMSO-d₆) δ ppm 8.90-8.92 (d, 1H), 7.48-7.49 (d, 2H), 7.32-7.41 (m, 4H), 7.18-7.20 (d, 2H), 7.10-7.12 (dd, 1H), 6.92-6.94 (d, 2H), 6.57-6.59 (d, 1H), 4.88 (s, 2H), 3.46 (s, 2H), 2.39 (s, 3H), 2.20 (s, 3H).

### Example 4

### N-(di-p-tolylmethyl)-2-(4-((3,5-dimethylisoxazol-4-yl)methoxy)phenyl)acetamide

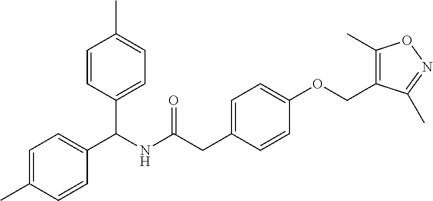 [SMILES: Cc1ccc(C(NC(=O)Cc2ccc(OCc3c(C)noc3C)cc2)c2ccc(C)cc2)cc1]

### (a) di-p-tolylmethanamine

This compound was synthesized from 4-methylbenzonitrile and p-tolylmagnesium bromide essentially as described in example 1 (e). LCMS-P1: 195 [M-NH₂]⁺; Rₜ: 1.217 min. ¹H NMR (400 MHz, CDCl₃) δ ppm 7.24 (d, J=7.6 Hz, 4H), 7.11 (d, J=8 Hz, 4H), 5.15 (s, 1H), 2.31 (s, 6H), 1.82 (br, 2H).

### (b) N(di-p-tolylmethyl)-2-(4-((3,5-dimethylisoxazol-4-yl)methoxy)phenyl)acetamide

To a stirred solution of 2-(4-((3,5-dimethylisoxazol-4-yl)methoxy)phenyl)acetic acid (261 mg, 1 mmol) in dichloromethane (10 mL) was added di-p-tolylmethanamine (211 mg, 1 mmol), DIPEA (258 mg, 2 mmol), EDC (230 mg, 1.2 mmol) and HOBt (162 mg, 1.2 mmol) successively. The resulting mixture was stirred at rt overnight. Additional dichloromethane (20 mL) was added, the resulting mixture was washed with (10 mL×3), 1% HCl (10 mL×3), and the organic layer was dried over Na₂SO₄. After removal of the organic solvent, the crude product was purified by preparatory HPLC using 10-100% water/acetonitrile with 0.1% TFA to give the title compound (275 mg, 61%). LCMS-P1: 455 [M+H]⁺; Rₜ: 1.753 min. ¹H NMR (500 MHz, CDCl₃) δ ppm 7.14 (d, J=8.0 Hz, 2H), 7.02 (d, J=8.0 Hz, 4H), 6.92 (d, J=8.2 Hz, 4H), 6.85 (d, J=8.5 Hz, 2H), 6.10 (d, J=8.5 Hz, 1H), 5.90 (d, J=8.5 Hz, 1H), 4.71 (s, 2H), 3.51 (s, 2H), 2.33 (s, 3H), 2.24 (s, 6H), 2.22 (s, 3H).

### Example 5

### N-((4-chlorophenyl)(2,4-dimethylphenyl)methyl)-2-(4-((3,5-dimethylisoxazol-4-yl)methoxy)phenyl)acetamide-d₁

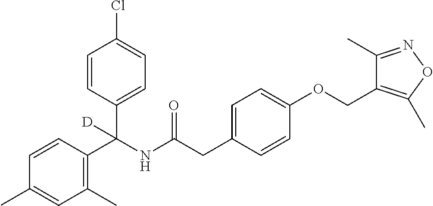 [SMILES: [2H]C(NC(=O)Cc1ccc(OCc2c(C)noc2C)cc1)(c1ccc(Cl)cc1)c1ccc(C)cc1C]

### (a) (4-chlorophenyl)(2,4-dimethylphenyl)methanamine-d₁

This compound was synthesized from 2,4-dimethylbenzonitrile and (4-chlorophenyl)magnesium bromide essentially as described in example 1 (e) except NaBD₄ was used. LCMS-T1: 230.1 [M-NH₂]⁺; Rₜ=4.50 min.

### (b) N-((4-chlorophenyl)(2,4-dimethylphenyl)methyl)-2-(4-((3,5-dimethylisoxazol-4-yl)methoxy)phenyl)acetamide-d₁

This compound was synthesized from (4-chlorophenyl)(2,4-dimethylphenyl)methanamine-d₁ and 2-(4-((3,5-dimethylisoxazol-4-yl)methoxy)phenyl)acetic acid essentially as described in example 4 (b). LCMS-T1 491.2 [M+H⁺]. ¹H NMR (DMSO-d₆) δ 8.81 (1H, d), 7.35 (2H, d), 7.17-7.14 (4H, m), 6.96-6.88 (5H, m), 4.85 (2H, s), 3.41 (2H, s), 2.36 (3H, s), 2.21 (3H, s), 2.17 (3H, s), 2.12 (3H, s).

### Example 6

### 2-(4-((3,5-dimethylisoxazol-4-yl)methoxy)phenyl)-N-(1-phenyl-1-(p-tolyl)ethyl)acetamide

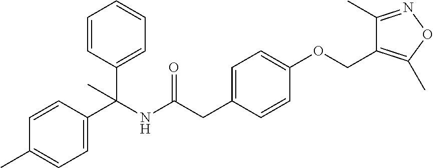 [SMILES: Cc1ccc(C(C)(NC(=O)Cc2ccc(OCc3c(C)noc3C)cc2)c2ccccc2)cc1]

2-(4-((3,5-dimethylisoxazol-4-yl)methoxy)phenyl)acetic acid (60 mg, 0.2 mmol) and 1-phenyl-1-(p-tolyl)ethanamine (24 mg, 0.1 mmol) were dissolved in dichloromethane (15 mL). HATU (349 mg, 0.9 mmol) was added followed by N-methyl morpholine (200 mg, 1.6 mmol) and the reaction was stirred overnight. Solvent was removed and the title compound was purified by reverse phase chromatography using acetonitrile/water with 0.05% TFA, (2 mg, 2%). LCMS-T1: 454.5 [M+H]⁺; Rₜ=5.74 min. ¹H NMR (400 MHz, CDCl₃) δ ppm 7.28 (m, 2H), 7.15 (d, 2H), 7.09 (d, 2H), 6.89 (t, 2H), 6.83 (d, 2H) 6.18 (d, 1H), 5.91 (d, 2H), 3.99 (s, 3H), 3.70 (s, 2H), 3.55 (s, 2H), 2.31 (s, 3H), 2.09 (s, 3H), 2.07 (s, 3H).

### Example 7

### N-((4-chlorophenyl)(2,4-dimethylphenyl)methyl)-2-(4-(2-(3,5-dimethylisoxazol-4-yl)ethoxy)phenyl)acetamide

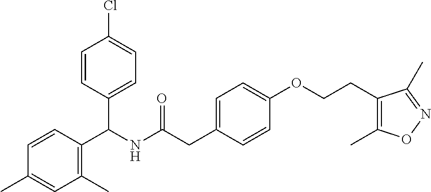 [SMILES: Cc1ccc(C(NC(=O)Cc2ccc(OCCc3c(C)noc3C)cc2)c2ccc(Cl)cc2)c(C)c1]

### (a) (4-chlorophenyl)(2,4-dimethylphenyl)methanamine

This compound was synthesized from 2,4-dimethylbenzonitrile and 4-chloro phenylmagnesiumbromide essentially as described in example 1 (e) and was purified by silica gel column chromatography (20% ethyl acetate in hexanes) to provide the title compound (1.20 g, 29.62%). ¹H NMR (400 MHz, DMSO-d₆) δ ppm 7.27-7.36 (m, 5H), 6.97-6.99 (d, 1H), 6.91 (d, 1H), 5.18 (s, 1H), 2.17-2.22 (d, 8H).

### (b) methyl 2-(4-(2-(3,5-dimethylisoxazol-4-yl)ethoxy)phenyl)acetate

To 2-(3,5-dimethylisoxazol-4-yl)ethanol (100 mg, 0.7 mmol) and triethylamine (0.12 mL, 0.8 mmol) in dichloromethane (5 mL) at 0° C., mesyl chloride (97 mg, 0.8 mmol) was added dropwise. The reaction was stirred for 1 h at 0° C. and then for 30 minutes at rt. When the reaction was complete, water (5 mL) was added and the mixture was extracted with dichloromethane. The organic layer was washed with saturated sodium bicarbonate and brine and dried over sodium sulfate. Solvent was removed and 2-(3,5-dimethylisoxazol-4-yl)ethyl methanesulfonate was dissolved in DMF (1 mL) and used crude. To methyl 2-(4-hydroxyphenyl)acetate (166 mg, 0.6 mmol) in DMF (2 mL), was added to sodium hydride (60%, 32 mg, 0.7 mmol) in DMF (2 mL) at 0° C. The reaction was stirred for 30 min at 0° C. and then for 30 minutes at rt, 2-(3,5-dimethylisoxazol-4-yl)ethyl methanesulfonate in DMF was added and the reaction was stirred for 30 min at rt and then at 90° C. overnight. Water (10 mL) was added and the mixture was extracted with ethyl acetate (2×15 mL), dried over sodium sulfate, and solvent was removed. Title compound was purified by silica gel column chromatography (0-18% ethyl acetate in hexanes) to provide (40 mg, 24%).

### (c) 2-(4-(2-(3,5-dimethylisoxazol-4-yl)ethoxy)phenyl)acetic acid

This compound was synthesized from methyl 2-(4-(2-(3,5-dimethylisoxazol-4-yl)ethoxy)phenyl)acetate essentially as described in example 1 (c). (800 mg, 80%). MS 275.9 [M+H]⁺;

### (d) N-((4-chlorophenyl)(2,4-dimethylphenyl)methyl)-2-(4-(2-(3,5-dimethylisoxazol-4-yl)ethoxy)phenyl)acetamide

This compound was synthesized from 2-(4-(2-(3,5-dimethylisoxazol-4-yl)ethoxy)phenyl)acetic acid and (4-chlorophenyl)(2,4-dimethylphenyl)methanamine essentially as described in example 1 (f) (0.023 g, 25.27%). LCMS-X1: 503.5 [M+H]⁺; Rₜ=5.52 min. ¹H NMR (400 MHz, DMSO-d₆) δ ppm 8.80-8.82 (d, 1H), 7.36-7.38 (d, 2H), 7.14-7.18 (t, 4H), 6.90-6.98 (m, 2H), 6.81-6.83 (d, 2H), 6.14-6.17 (d, 1H), 3.97-4.00 (t, 2H), 3.39-3.42 (d, 2H), 2.72-2.75 (t, 2H), 2.32 (s, 3H), 2.23 (s, 3H), 2.20 (s, 3H), 2.14 (s, 3H)

### Example 8

### N-((4-chlorophenyl)(phenyl)methyl)-2-(4-(((3,5-dimethylisoxazol-4-yl)methyl)(methyl)amino)phenyl)acetamide

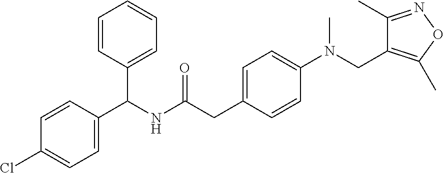 [SMILES: Cc1noc(C)c1CN(C)c1ccc(CC(=O)NC(c2ccccc2)c2ccc(Cl)cc2)cc1]

To a solution of N-((4-chlorophenyl)(phenyl)methyl)-2-(4-(((3,5-dimethylisoxazol-4-yl)methyl)amino)phenyl)acetamide (0.200 g, 0.435 mmol) in acetone (10 mL) at 25° C. was added potassium carbonate (0.151 g, 1.08 mmol) followed by addition of iodomethane (0.123 g, 0.87 mmol) at 0° C. and the reaction was allowed to stir for 24 h. Then the solvent was evaporated under reduced pressure and water (10 mL) was added. The solution was extracted with ethyl acetate (3×25 mL) and the combined organic layers were washed with water (20 mL), brine (25 mL), dried over Na₂SO₄, and evaporated under reduced pressure to obtain a crude product which was purified using preparative TLC on silica gel using 40% ethyl acetate in hexanes to provide the title compound was isolated. (22 mg, 10.67%). LCMS-X1: 474.6 [M+H]⁺; Rₜ=6.78 min. ¹H NMR (400 MHz, DMSO-d₆) δ ppm 8.92-8.94 (d, 1H), 7.34-7.39 (d, 2H), 7.24-7.32 (m, 7H), 7.09-7.11 (d, 2H), 6.76-6.79 (d, 2H), 6.07-6.09 (d, 1H), 4.19 (d, 2H), 3.36 (s, 2H), 2.69 (s, 3H), 2.33 (s, 3H), 2.05 (s, 3H).

### Example 9

### N-((4-chlorophenyl)(phenyl)methyl)-2-(4-(thiazol-4-yl)methoxy)phenyl)acetamide

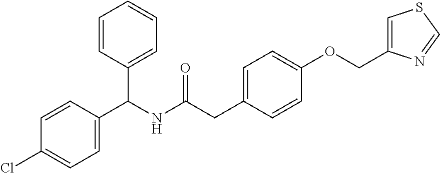 [SMILES: O=C(Cc1ccc(OCc2cscn2)cc1)NC(c1ccccc1)c1ccc(Cl)cc1]

### (a) N-((4-chlorophenyl)(phenyl)methyl)-2-(4-hydroxyphenyl)acetamide

This compound was synthesized from 2-(4-hydroxyphenyl)acetic acid and (4-chlorophenyl)(phenyl)methanamine essentially as described in example 1 (f) and the product was purified by silica gel column chromatography (40% EtOAc/hexanes) to provide the title compound (4.5 g, 65.2%). ¹H NMR (400 MHz, DMSO-d₆) δ ppm 9.25 (s, 1H), 8.94-8.96, (d, 1H), 3.37-3.39 (d, 2H), 7.28-7.34 (m, 3H), 7.24-7.26 (m 3H), 7.04-7.06 (d, 2H), 6.65-6.67 (d, 2H), 6.07-6.09 (d, 1H), 4.08 (s, 2H). MS (ESI+) 352.0 (M+H).

### (b) N-((4-chlorophenyl)(phenyl)methyl)-2-(4-(thiazol-4-yl)methoxy)phenyl)acetamide

To a solution of N-((4-chlorophenyl)(phenyl)methyl)-2-(4-hydroxyphenyl)acetamide (0.300 g, 0.85 mmol) in DMF (4.0 mL) was added potassium carbonate (0.353 g, 0.2.55 mmol) and 4-(chloromethyl) thiazole (0.136 g, 1.02 mmol), and the reaction was stirred for 20 h. After completion of the reaction, the reaction mixture was poured into water (20 mL) and extracted with ethyl acetate (100 mL). The organic layer was dried over Na₂SO₄ and concentrated. The compound was purified by silica gel column chromatography (32% EtOAc/hexanes) to provide the title compound (0.185 g, 48.42%). LCMS-X1: 449.1 [M+H]⁺; Rₜ=5.58 min. ¹H NMR (400 MHz, DMSO-d₆) δ ppm 9.12-9.13 (d, 1H), 9.00-9.02 (d, 1H), 7.77 (s, 1H), 7.37-7.40 (d, 2H), 7.25-7.35 (m, 7H), 6.95-6.97 (d, 2H), 6.07-6.09 (s, 1H), 5.18 (s, 2H), 3.47 (s, 2H).

### Example 10

### N-((4-chlorophenyl)(2,4-dimethylphenyl)methyl)-2-(4-((3-ethyl-5-methylisoxazol-4-yl)methoxy)phenyl)acetamide

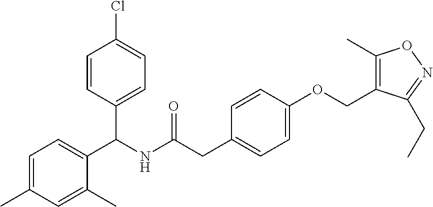 [SMILES: CCc1noc(C)c1COc1ccc(CC(=O)NC(c2ccc(Cl)cc2)c2ccc(C)cc2C)cc1]

### (a) (4-chlorophenyl)(2,4-dimethylphenyl)methanamine

This compound was synthesized from 2,4-dimethylbenzonitrile and p-chloromagnesium bromide essentially as described in example 1 (e). (1.20 g, 29.62%). ¹H NMR (400 MHz, DMSO-d₆) δ ppm 7.27-7.36 (m, 5H), 6.97-6.99 (d, 1H), 6.91 (d, 1H), 5.18 (s, 1H), 2.17-2.22 (d, 8H).

### (b) N-((4-chlorophenyl)(2,4-dimethylphenyl)methyl)-2-(4-hydroxyphenyl)acetamide

This compound was synthesized from 2-(4-hydroxyphenyl)acetic acid and (4-chlorophenyl)(2,4-dimethylphenyl)methanamine essentially as described in example 4 (b). The product was purified by silica gel column chromatography (25% ethyl acetate/hexanes) to provide the title compound (0.81 g, 43.78%) MS (ESI+) 380.1 (M+H). ¹H NMR (400 MHz, DMSO-d₆) δ ppm 9.23 (s, 1H), 8.79-8.81 (d, 1H), 7.37-7.39 (d, 2H), 7.16-7.18 (d, 2H), 7.03-7.05 (d, 2H), 6.97-6.99 (d, 1H), 6.91-6.95 (t, 2H), 6.65-6.67 (d, 2H), 6.17-6.15 (d, 1H), 3.33-3.37 (t, 2H), 2.14-2.23 (m, 7H).

### (c) (3-ethyl-5-methylisoxazol-4-yl)methanol

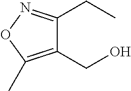

To a solution ethyl 3-ethyl-5-methylisoxazole-4-carboxylate (0.50 g, 2.72 mmol) in anhydrous THF (7 mL) at −78° C. was added 1.0 M solution of LiAlH₄ (4.00 mL, 4.08 mmol) dropwise. After completion of the addition, the reaction mixture was warmed to −40° C. and stirred for 1 h. After completion of the reaction, the reaction mixture was warmed to −10° C. and water (5 mL) was added and the mixture was extracted with ethyl acetate (2×15 mL). The combined organic layers were dried over Na₂SO₄ and concentrated to provide the title compound (0.325 g, 84.63%) MS (ESI+) 142.1 (M+H). ¹H NMR (400 MHz, DMSO-d₆) δ ppm 4.91 (s, 1H), 4.26 (s, 2H), 2.58-2.67 (m, 2H), 2.32 (s, 3H), 1.17-1.20 (t, 3H).

### (d) 4-(chloromethyl)-3-ethyl-5-methylisoxazole

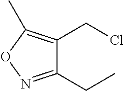

To a solution (3-ethyl-5-methylisoxazol-4-yl)methanol (0.10 g, 0.85 mmol) in dichloromethane (4 mL) at 0° C. was added SOCl₂ (0.09 mL, 0.12 mmol) dropwise. After completion of the addition, the reaction mixture was warmed to rt and stirred for 2 h. After completion of the reaction, solvent was evaporated and azeotroped by dichloromethane (2×10 mL) to provide the title compound (0.11 g, 97.34%). ¹H NMR (400 MHz, DMSO-d₆) δ ppm 4.68 (s, 2H), 2.64-2.69 (q, 2H), 2.40 (s, 3H), 1.20-1.23 (t, 3H). MS 160.1 (M+H).

### (e) N-((4-chlorophenyl)(2,4-dimethylphenyl)methyl)-2-(4-((3-ethyl-5-methylisoxazol-4-yl)methoxy)phenyl)acetamide

To a solution of N-((4-chlorophenyl)(2,4-dimethylphenyl)methyl)-2-(4-hydroxyphenyl)acetamide (0.20 g, 0.52 mmol) in anhydrous DMF (5 mL) at 25° C. was added cesium carbonate (0.51 g, 1.57 mmol) and 4-(chloromethyl)-3-ethyl-5-methylisoxazole (0.085 g, 0.52 mmol). The reaction was stirred for 4 h at 90-100° C. under argon atmosphere. After completion of the reaction, water (20 mL) was added and extracted with ethyl acetate (2×35 mL). The combined organic layers were dried over Na₂SO₄ and concentrated and the product was purified by silica gel column chromatography (18% ethyl acetate/hexanes) to provide the title compound (0.095 g, 35.98%). LCMS-X1: 504.1 [M+H]⁺; Rₜ=5.61 min. ¹H NMR (400 MHz, DMSO-d₆) δ ppm 8.82-8.84 (d, 1H), 7.36-7.39 (d, 2H), 7.16-7.19 (q, 4H), 6.91-6.99 (m, 5H), 6.16-6.18 (d, 1H), 4.90 (s, 2H), 3.44 (s, 2H), 2.60-2.66 (m, 2H), 2.39 (s, 3H), 2.23 (s, 3H), 2.15 (s, 3H), 1.16-1.20 (t, 3H).

### Example 11

### N-((4-chlorophenyl)(phenyl)methyl)-2-(4-((3-(hydroxymethyl)-5-methylisoxazol-4-yl)methoxy)phenyl)acetamide

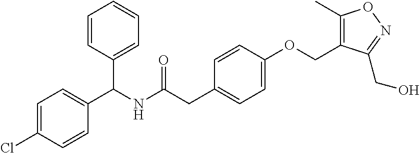 [SMILES: Cc1onc(CO)c1COc1ccc(CC(=O)NC(c2ccccc2)c2ccc(Cl)cc2)cc1]

### (a) ethyl 4-((4-(2-((4-chlorophenyl)(phenyl)methylamino)-2-oxoethyl)phenoxy)methyl)-5-methylisoxazole-3-carboxylate

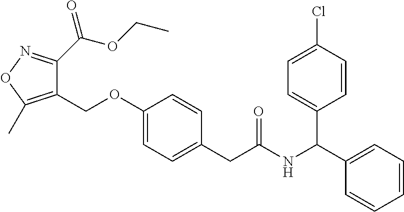 [SMILES: CCOC(=O)c1noc(C)c1COc1ccc(CC(=O)NC(c2ccccc2)c2ccc(Cl)cc2)cc1]

To a solution of N-((4-chlorophenyl)(phenyl)methyl)-2-(4-hydroxyphenyl)acetamide (0.259 g, 0.73 mmol) in dimethylformamide (5 mL) was added ethyl 4-(chloromethyl)-5-methylisoxazole-3-carboxylate (0.150 g, 0.73 mmol) and anhydrous potassium carbonate (0.304 g, 2.20 mmol), and the reaction was refluxed for 2 h. After completion of the reaction, water (30 mL) was added into the reaction mixture and the mixture was extracted with ethyl acetate (125 mL). The organic layer was washed with brine (25 mL), dried over Na₂SO₄, and evaporated to obtain a crude product which was purified using silica gel column chromatography using 32% EtOAc:hexanes to obtain the title compound (0.310 g, 81.3%) as a light yellow semi solid. MS (ESI+) 519.4 (M+H).

### (b) N-((4-chlorophenyl)(phenyl)methyl)-2-(4-((3-(hydroxymethyl)-5-methylisoxazol-4-yl)methoxy)phenyl)acetamide

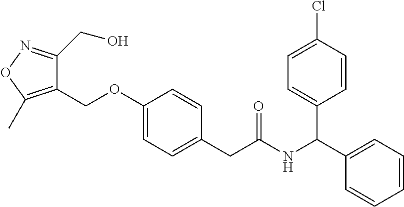 [SMILES: Cc1onc(CO)c1COc1ccc(CC(=O)NC(c2ccccc2)c2ccc(Cl)cc2)cc1]

To a solution ethyl 4-((4-(2-((4-chlorophenyl)(phenyl)methylamino)-2-oxoethyl)phenoxy)methyl)-5-methylisoxazole-3-carboxylate (0.300 g, 0.579 mmol) in THF (5 mL) at 0° C. was added 1 M lithium aluminum hydride solution in THF (0.57 mL, 0.579 mmol) and the reaction was stirred at 0° C. for 1 h. After completion of the reaction, crushed ice was added to the reaction mixture and extracted with ethyl acetate (300 mL). The combined organic layers were dried over Na₂SO₄ and concentrated to obtain a crude product which was purified by preparative TLC on silica gel using (50% EtOAc/hexanes) as mobile phase to provide the title compound (0.075 g, 51.7%). LCMS-X1: 477.3 [M+H]⁺; Rₜ=6.32 min. ¹H NMR (400 MHz, DMSO-d₆) δ ppm 8.99-9.01 (d, 1H), 7.38-7.40 (d, 2H), 7.24-7.35 (m, 7H), 7.17-7.20 (m, 2H), 6.91-6.93 (d, 2H), 6.07-6.10 (d, 1H), 5.42-5.45 (t, 1H), 4.93 (s, 2H), 4.51-4.52 (d, 2H), 3.47 (s, 2H), 2.40 (s, 3H).

### Example 12

### N-((4-chlorophenyl)(phenyl)methyl)-2-(4-((5-methylisoxazol-3-yl)methoxy)phenyl)acetamide

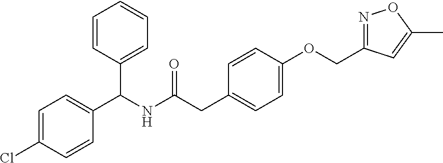 [SMILES: Cc1cc(COc2ccc(CC(=O)NC(c3ccccc3)c3ccc(Cl)cc3)cc2)no1]

To a solution of N-((4-chlorophenyl)(phenyl)methyl)-2-(4-hydroxyphenyl)acetamide (0.250 g, 0.710 mmol) in DMF (2.0 mL) was added 60% sodium hydride (0.034 g, 0.85 mmol) at 0° C. and stirred for 20 min. 3-(chloromethyl)-5-methylisoxazole (0.112 g, 0.852 mmol) was added and stirred for h. After completion of the reaction, the reaction mixture was poured into crushed ice and extracted with ethyl acetate (100 mL). The organic layer was dried over Na₂SO₄ and concentrated and product was purified by silica gel column chromatography (40% EtOAc/hexanes) to provide the title compound (0.080 g, 25.23%). LCMS-X1: 447.1 [M+H]⁺; Rₜ=5.60 min. ¹H NMR (400 MHz, DMSO-d₆) δ ppm 9.00-9.02 (d, 1H), 7.38-7.40 (d, 2H), 7.33-7.34 (d, 2H), 7.24-7.33 (m, 4H), 7.18-7.20 (d, 2H), 6.92-6.94 (d, 2H), 6.31 (s, 1H), 6.07-6.09 (d, 1H), 5.10 (s, 2H), 3.47 (s, 2H), 2.40 (s, 3H).

### Example 13

### N-((4-chlorophenyl)(phenyl)methyl)-2-(4-((3,5-dimethylisoxazol-4-yl)methoxy)phenyl)-N-methylacetamide

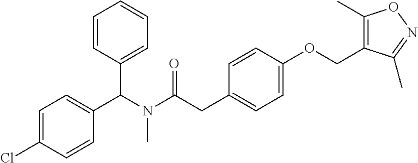 [SMILES: Cc1noc(C)c1COc1ccc(CC(=O)N(C)C(c2ccccc2)c2ccc(Cl)cc2)cc1]

### (a) 1-(4-chlorophenyl)-N-methyl-1-phenylmethanamine

This compound was synthesized from (4-chlorophenyl)(phenyl)methanamine hydrochloride essentially as described in example 8 and was purified by silica gel column chromatography (10% EtOAc/hexane) to provide the title compound (0.11 g, 40.44%). ¹H NMR (400 MHz, DMSO-d₆) δ ppm 7.39-7.43 (t, 3H), 7.32-7.37 (t, 4H), 7.26-7.30 (t, 2H), 7.16-7.22 (t, 1H), 4.65 (s, 1H), 2.12 (s, 3H).

### (b) N-((4-chlorophenyl)(phenyl)methyl)-2-(4-((3,5-dimethylisoxazol-4-yl)methoxy)phenyl)-N-methylacetamide

This compound was synthesized from 2-(4-((3,5-dimethylisoxazol-4-yl)methoxy)phenyl)acetic acid and 1-(4-chlorophenyl)-N-methyl-1-phenylmethanamine essentially as described in example 3 (c). (0.184 g, 50.68%). LCMS-X1: 475.2 [M+H]⁺; Rₜ=5.95 min. ¹H NMR (400 MHz, DMSO-d₆) δ ppm 7.32-7.44 (m 6H), 7.04-7.17 (m, 6H), 6.91-6.96 (m, 3H), 4.89 (s 2H), 3.77 (s, 2H), 2.75 (s, 2H), 2.39 (s, 3H) 2.20 (s 3H).

### Example 14

### 2-(4-((3,5-dimethylisoxazol-4-yl)methoxy)phenyl)-N-(phenyl(o-tolyl)methyl)acetamide

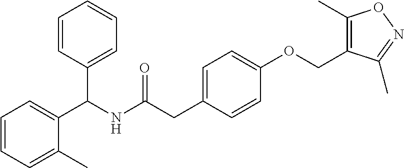 [SMILES: Cc1ccccc1C(NC(=O)Cc1ccc(OCc2c(C)noc2C)cc1)c1ccccc1]

### (a) phenyl(o-tolyl)methanamine

This compound was synthesized from 2-methylbenzonitrile and phenylmagnesium chloride essentially as described in example 1 (e). (360 mg, 43%). LCMS-P1: 181 [M-NH₂]⁺; Rₜ: 1.604 min. ¹H NMR (500 MHz, CDCl₃) δ ppm 7.59-7.18 (m, 9H), 5.41 (s, 1H), 2.30 (s, 3H), 1.84 (s, 2H).

### (b) 2-(4-hydroxyphenyl)-N-(phenyl(o-tolyl)methyl)acetamide

This compound was synthesized from 2-(4-hydroxyphenyl)acetic acid and phenyl(o-tolyl)methanamine essentially as described in example 4 (b) and this the title compound was used directly without further purification. LCMS-P1: 332 [M+H]⁺; Rₜ: 1.544 min.

### (c) 2-(4-((3,5-dimethylisoxazol-4-yl)methoxy)phenyl)-N-(phenyl(o-tolyl)methyl)acetamide

To a stirred solution of 2-(4-hydroxyphenyl)-N-(phenyl(o-tolyl)methyl)acetamide (400 mg, 1.21 mmol) in 10 mL of DMF was added 4-(chloromethyl)-3,5-dimethylisoxazole (176 mg, 1.21 mmol), K₂CO₃ (334 mg, 2.42 mmol), and tetrabutylammonium iodide successively. The reaction mixture was stirred at rt overnight. Water (20 mL) was added and the reaction was extracted with ethyl acetate (30 mL×3). The combined organics were washed with brine and dried over Na₂SO₄. After removal of the organic solvent, the crude product was purified by silica gel column chromotography (petroleum ether/EtOAc=3/1) to give 262 mg product (49%). The product was repurified by preparatory HPLC using 10-100% water/acetonitrile with 0.1% TFA to obtain the title compound (156 mg, 29%). LCMS-P1: 441 [M+H]⁺; Rₜ: 1.696 min. ¹H NMR (500 MHz, CDCl₃) δ ppm 7.29-6.91 (m, 13H), 6.40 (d, J=10.0 Hz, 1H), 5.96 (d, J=10.0 Hz, 1H), 4.78 (s, 2H), 3.59 (s, 2H), 2.40 (s, 3H), 2.29 (s, 3H), 2.24 (s, 3H).

### Example 15

### N-((4-chlorophenyl)(phenyl)methyl)-2-(4-(oxazol-5-ylmethoxy)phenyl)acetamide

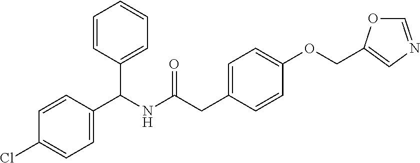 [SMILES: O=C(Cc1ccc(OCc2cnco2)cc1)NC(c1ccccc1)c1ccc(Cl)cc1]

To a solution of N-((4-chlorophenyl)(phenyl)methyl)-2-(4-hydroxyphenyl)acetamide (0.423 g, 1.20 mmol), in THF (1.0 mL) was added oxazol-5-ylmethanol (0.150 g, 1.50 mmol) and triphenylphosphine (0.409 g, 1.56 mmol) and the reaction was sonicated in a 33-KHz sonicating bath at 0° C. until a clear solution was obtained. To the reaction mixture, di-isopropyl azodicarboxylate (0.315 g, 1.56 mmol) was added dropwise over a period of 5 minutes at 0° C. and sonicated for 15 min. The THF was removed under vacuum and then remaining mixture was poured into water (20 mL) and extracted with dichloromethane (100 mL). The organic layer was dried over Na₂SO₄ and concentrated and the product was purified by silica gel column chromatography (40% EtOAc/hexanes) and again repurified by preparative TLC on silica gel (80% EtOAc/hexanes) to provide the title compound. (0.128 g, 24.6%). LCMS-X1: 433.1 [M+H]⁺; Rₜ=5.01 min. ¹H NMR (400 MHz, DMSO-d₆) δ ppm 8.99-9.02 (d, 1H), 8.41 (s, 1H), 7.38-7.40 (d, 2H), 7.29-7.35 (m, 3H), 7.24-7.27 (m, 5H), 7.18-7.20 (d, 2H), 6.94-6.96 (d, 2H), 6.08-6.10 (d, 1H), 5.14 (s, 2H), 3.47 (s, 2H).

### Example 16

### 2-(((3,5-dimethylisoxazol-4-yl)methyl)(4-(2-oxo-2-((phenyl(p-tolyl)methyl)amino)ethyl)phenyl)amino)acetic acid

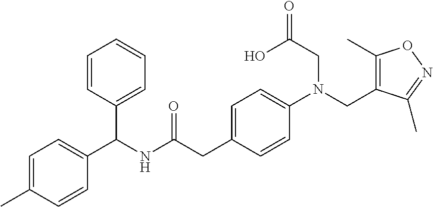 [SMILES: Cc1ccc(C(NC(=O)Cc2ccc(N(CC(=O)O)Cc3c(C)noc3C)cc2)c2ccccc2)cc1]

### (a) 2-(4-(((3,5-dimethylisoxazol-4-yl)methyl)amino)phenyl)-N-(phenyl(p-tolyl)methyl)acetamide

This compound was synthesized from 2-(4-(((3,5-dimethylisoxazol-4-yl)methyl)amino)phenyl)acetic acid and phenyl(p-tolyl)methanamine essentially as described in example 4 (b) and purified by silica gel column chromatography using 0-100% ethyl acetate/hexane (118 mg, 12%). LCMS-T1: 440.3 [M+H]⁺; Rₜ: 5.53 min.

### (b) methyl 2-(((3,5-dimethylisoxazol-4-yl)methyl)(4-(2-oxo-2-((phenyl(p-tolyl)methyl)amino)ethyl)phenyl)amino)acetate

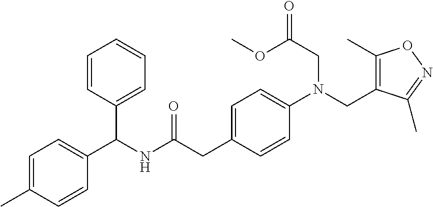 [SMILES: COC(=O)CN(Cc1c(C)noc1C)c1ccc(CC(=O)NC(c2ccccc2)c2ccc(C)cc2)cc1]

2-(4-(((3,5-dimethylisoxazol-4-yl)methyl)amino)phenyl)-N-(phenyl(p-tolyl)methyl)acetamide (118 mg, 0.3 mmol), methyl 2-bromoacetate (41 mg, 0.3 mmol), tetrabutylammonium iodide (198 mg, 0.5 mmol), and cesium carbonate (174 mg, 0.5 mmol) in DMF (1 mL) were stirred overnight. The reaction was filtered to remove solids. Water was added and the solution was purified by reverse phase using 10-90% acetonitrile/water with 0.05% TFA to give the title compound (130 mg, 94%). LCMS-T1: 512.2 [M+H]⁺; Rₜ: 6.30 min.

### (c) 2-(((3,5-dimethylisoxazol-4-yl)methyl)(4-(2-oxo-2-((phenyl(p-tolyl)methyl)amino)ethyl)phenyl)amino)acetic acid

Methyl 2-(((3,5-dimethylisoxazol-4-yl)methyl)(4-(2-oxo-2-((phenyl(p-tolyl)methyl)amino)ethyl)phenyl)amino)acetate (130 mg, 0.3 mmol) was stirred for 3 hours in aqueous 1 M LiOH (1.5 mL) and dioxane (6 mL). The reaction was acidified and the solution was purified by reversed phase chromatography using 10-90% acetonitrile/water with 0.05% TFA to give the title compound (130 mg, 94%). LCMS-T1: 498.3 [M+H]⁺; Rₜ: 5.74 min. ¹H NMR (400 MHz, MeOH-d₄) δ ppm 7.30-7.05 (m, 11H), 6.80 (d, 2H), 6.10 (t, 1H), 4.38 (s, 2H), 3.97 (s, 2H), 3.58 (s, 2H), 2.31 (s, 6H), 2.17 (s, 3H).

### Example 17

### N-(bis(4-fluorophenyl)methyl)-2-(4-((3-(hydroxymethyl)-5-methylisoxazol-4-yl)methoxy)phenyl)-2-methylpropanamide

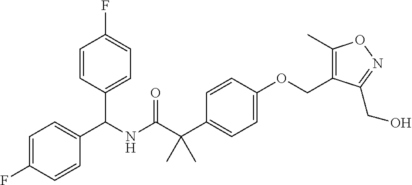 [SMILES: Cc1onc(CO)c1COc1ccc(C(C)(C)C(=O)NC(c2ccc(F)cc2)c2ccc(F)cc2)cc1]

### (a) methyl 2-(4-methoxyphenyl)acetate

This compound was synthesized from 2-(4-methoxyphenyl)acetic acid essentially as described in example 1 (a) (4.0 g, 92.37%). ¹H NMR (400 MHz, CDCl₃) δ ppm 7.17-7.19 (d, 2H), 6.87-6.89 (d, 2H), 3.73 (s, 3H), 3.60 (s, 5H).

### (b) methyl 2-(4-methoxyphenyl)-2-methylpropanoate

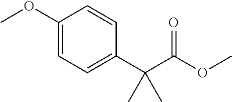

To a solution of methyl 2-(4-methoxyphenyl)acetate (4.0 g, 22.22 mmol) in THF (25 mL), was added iodomethane (9.45 g, 66.66 mmol) and the reaction mixture was cooled to −60° C., followed by addition of potassium tert-butoxide (7.48 g, 66.66 mmol) portion wise. The reaction mixture was stirred for 30 minutes at the same temperature. After completion of the reaction, water (30 mL) was added into the reaction mixture at −60° C. and extracted with ethyl acetate (2×250 mL). The combined organic layers were dried over Na₂SO₄ and concentrated to obtain a crude product which was purified by silica gel column chromatography using 2.5% EtOAc/hexanes to provide the title compound (3.52 g, 75.59%).

### (c) 2-(4-hydroxyphenyl)-2-methylpropanoic acid

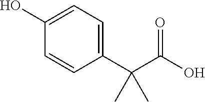

A solution of methyl 2-(4-methoxyphenyl)-2-methylpropanoate (3.50 g, 16.80 mmol) in dichloromethane (20 mL) was cooled to −78° C. and boron tribromide was added (1.0 M solution in dichloromethane, 50.4 mL, 50.40 mmol) over 30 minutes. After completion of addition, the temperature was raised to 0° C. and the reaction mixture was stirred for 30 minutes. After completion of the reaction, water was slowly added to the reaction mixture. The organic layer was separated, dried over Na₂SO₄, and concentrated to provide the title compound (1.4 g, 77.40%). ¹H NMR (400 MHz, DMSO-d₆) δ ppm 12.10 (br, 1H), 9.35 (s, 1H), 7.08-7.15 (dd, 2H), 6.68-6.71 (dd, 2H), 1.41 (s, 6H).

### (d) bis(4-fluorophenyl)methanamine

This compound was synthesized from 4-fluorobenzonitrile and (4-fluorophenyl)magnesium bromide essentially as described in example 1 (e). (0.450 g, 12.46%). ¹H NMR (400 MHz, DMSO-d₆) δ ppm 7.30-7.46 (m, 4H), 7.03-7.16 (m, 4H), 5.15 (s, 1H).

### (e) N-(bis(4-fluorophenyl)methyl)-2-(4-hydroxyphenyl)-2-methylpropanamide

To a solution of 2-(4-hydroxyphenyl)-2-methylpropanoic acid (0.18 g, 0.19 mmol) in dimethylformamide (2 mL), EDC (0.228 g, 1.18 mmol), HOBt (0.181 g, 1.18 mmol), DMAP (0.242 g, 1.98 mmol) and bis(4-fluorophenyl)methanamine (0.218 g, 0.021 mmol) was added and the reaction mixture was stirred overnight at it. After completion of the reaction, water (50 mL) was added to the reaction mixture and extracted with ethyl acetate (100 mL). The organic layer was dried over Na₂SO₄ and concentrated to obtain a crude product which was purified by silica gel column chromatography (1.5% methanol in dichloromethane) to provide the title compound (200 mg, 52.49%). ¹H NMR (400 MHz, DMSO-d₆) δ ppm 9.24-9.26 (d, 1H), 7.99-8.02 (d, 1H), 7.16-7.19 (m, 4H), 7.06-7.18 (m, 5H), 6.66-6.68 (d, 2H), 6.18-6.20 (d, 1H), 1.43 (s, 6H).

### (f) ethyl 4-((4-(1-(bis(4-fluorophenyl)methylamino)-2-methyl-1-oxopropan-2-yl)phenoxy)methyl)-5-methylisoxazole-3-carboxylate

This compound was synthesized from N-(bis(4-fluorophenyl)methyl)-2-(4-hydroxyphenyl)-2-methylpropanamide and ethyl 4-(chloromethyl)-5-methylisoxazole-3-carboxylate essentially as described in example 11(a) (0.200 g, 69.7%) as a light yellow semi solid.

### (g) N-(bis(4-fluorophenyl)methyl)-2-(4-((3-(hydroxymethyl)-5-methylisoxazol-4-yl)methoxy)phenyl)-2-methylpropanamide

This compound was synthesized from ethyl 4-((4-(1-(bis(4-fluorophenyl)methylamino)-2-methyl-1-oxopropan-2-yl)phenoxy)methyl)-5-methylisoxazole-3-carboxylate essentially as described in example 11(b). (44 mg, 23.8%). LCMS-X1: 507.6 [M+H]⁺; Rₜ=6.59 min. ¹H NMR (400 MHz, DMSO-d₆) δ ppm 8.07-8.10 (d, 1H), 7.15-7.21 (m, 6H), 7.09-7.13 (m, 4H), 6.91-6.93 (d, 2H), 6.18-6.20 (d, 1H), 5.43-5.45 (t, 1H), 4.96 (s, 2H), 4.52-4.53 (d, 2H), 2.40 (s, 3H), 1.46 (s, 6H).

### Example 18

### N-((4-chlorophenyl)(phenyl)methyl)-2-(4-((3,5-dimethylisoxazol-4-yl)methoxy)phenyl)-2-methylpropanamide

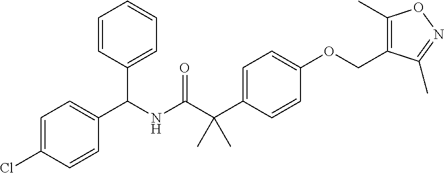 [SMILES: Cc1noc(C)c1COc1ccc(C(C)(C)C(=O)NC(c2ccccc2)c2ccc(Cl)cc2)cc1]

### (a) methyl 2-(4-((3,5-dimethylisoxazol-4-yl)methoxy)phenyl)-2-methylpropanoate

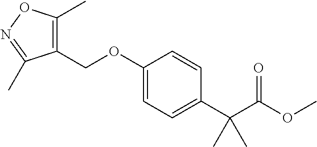 [SMILES: COC(=O)C(C)(C)c1ccc(OCc2c(C)noc2C)cc1]

To a solution of methyl 2-(4-((3,5-dimethylisoxazol-4-yl)methoxy)phenyl)acetate (0.5 g, 1.818 mmol) in THF (25 mL) at −78° C., iodomethane (0.34 mL, 5.454 mmol) was added dropwise. To this solution, potassium tert-butoxide (0.627 g, 5.454 mmol) was added portion wise over 30 min and the reaction was stirred at −78° C. for 1 h followed by rt for another one hour. After completion of the reaction, the reaction mixture was quenched by addition of ammonium chloride solution in water (25 mL) and extracted with (2×50 mL) ethyl acetate. The organic layers were dried (Na₂SO₄) and concentrated to obtain crude the title compound (0.60 g, 91.66%). MS (ESI+) 304.2 (M+H),

### (b) 2-(4-((3,5-dimethylisoxazol-4-yl)methoxy)phenyl)-2-methylpropanoic acid

This compound was synthesized from methyl 2-(4-((3,5-dimethylisoxazol-4-yl)methoxy)phenyl)-2-methylpropanoate essentially as described in example 1 (c). (0.350 g, 61.2%). MS (ESI+) 290.2 (M+H). ¹H NMR (400 MHz, DMSO-d₆) δ ppm 12.33 (s, 1H), 7.26-7.28 (d, 2H), 6.94-6.97 (d, 2H), 4.88 (s, 2H), 2.39 (s, 3H), 2.20 (s, 3H), 1.42 (s, 6H).

### (c) N-((4-chlorophenyl)(phenyl)methyl)-2-(4-((3,5-dimethylisoxazol-4-yl)methoxy)phenyl)-2-methylpropanamide

To a solution of 2-(4-((3,5-dimethylisoxazol-4-yl)methoxy)phenyl)-2-methylpropanoic acid (0.20 g, 0.692 mmol) in tetrahydrofuran (35 mL), EDC (0.2 g, 1.41 mmol) was added portion wise and the reaction was stirred at rt for 2 h. (4-chlorophenyl)(phenyl)methanamine hydrochloride (0.21 g, 0.826 mmol), HOBt (0.105 g, 0.686 mmol), and triethylamine (0.287 mL, 2.074 mmol) were then added, and the reaction mixture was stirred for 24 h at rt. After completion of the reaction, the reaction mixture was quenched by addition of water (50 mL) and extracted with 2×50 mL ethyl acetate. The organic layers were dried (Na₂SO₄) and concentrated to obtain a crude product which was purified by silica gel column chromatography (35% EtOAc/hexane) to provide the title compound (0.074 g, 21.95%). MS (ESI+) 489.3 (M+H). ¹H NMR (400 MHz, CDCl₃) δ ppm 7.25-7.31 (m, 8H), 7.03-7.04 (m, 4H), 6.92-6.99 (m, 2H), 6.16-6.18 (d, 1H), 5.72-5.74 (d, 1H), 4.80 (s, 2H), 2.43 (s, 3H), 2.31 (s, 3H), 1.59-1.63 (d 6H).

### Example 19

### 2-(4-((3,5-diisopropylisoxazol-4-yl)methoxy)phenyl)-N-((2,4-dimethylphenyl)(phenyl)methyl)acetamide

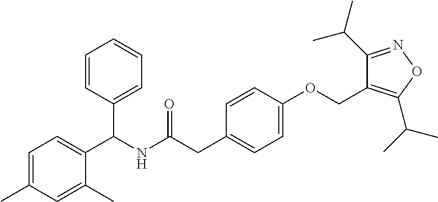 [SMILES: Cc1ccc(C(NC(=O)Cc2ccc(OCc3c(C(C)C)noc3C(C)C)cc2)c2ccccc2)c(C)c1]

### (a) ethyl 2-isobutyryl-4-methyl-3-oxopentanoate

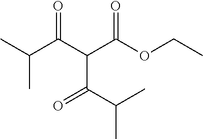 [SMILES: CCOC(=O)C(C(=O)C(C)C)C(=O)C(C)C]

A solution of ethyl 4-methyl-3-oxopentanoate (5.00 g, 31.6 mmol) in dry benzene (5.0 mL) was added to a mixture of Na-metal (0.80 g, 34.7 mmol) in dry benzene (50 mL) dropwise within 10 minutes at rt. After completion of the addition, the reaction mixture was stirred at rt until Na metal was dissolved and the reaction was refluxed for 1 h. After 1 h, the reaction mixture was cooled to 0° C. and isobutyryl chloride (3.70 g 34.7 mmol) was added dropwise at 0° C. The reaction mixture was stirred at 0° C. for 5 min. and then heated to reflux and stirred at reflux for 3 h. The reaction mixture was then cooled to rt and stirred overnight. After completion of the reaction, water was added and the reaction was extracted with ethyl acetate (2×250 mL). The combined organic layers were dried using Na₂SO₄ and concentrated. The compound was purified by silica gel column chromatography using 100% hexanes to provide the title compound (3.52 g 48.88%).

### (b) ethyl 3,5-diisopropylisoxazole-4-carboxylate

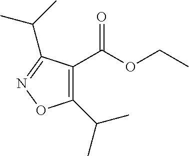 [SMILES: CCOC(=O)c1c(C(C)C)noc1C(C)C]

To a solution of ethyl 2-isobutyryl-4-methyl-3-oxopentanoate (2.80 g, 12.28 mmol) in Ethanol (10 mL) at 25° C. was added hydroxylamine hydrochloride (1.49 g, 21.49 mmol) in water (5 mL) and the resulting mixture was refluxed for 1 h. After 1 h, water (10 mL) was added into the reaction mixture and the reaction mixture was extracted with diethyl ether (3×50 mL). The combined organic layers were washed with 1 N NaOH solution (3×10 mL). The organic layer was dried using Na₂SO₄ and concentrated. The compound was purified by silica gel column chromatography eluting 2% EtOAC:hexanes to provide the title compound (1.80 g 65.21%), ¹H NMR (400 MHz, CDCl₃) δ ppm 4.31-4.36 (q, 2H), 3.73-3.80 (m, 1H), 3.42-3.49 (m, 1H), 1.27-1.38 (m, 15H).

### (c) (3,5-diisopropylisoxazol-4-yl)methanol

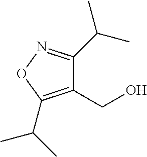

To a solution of ethyl 3,5-diisopropylisoxazole-4-carboxylate (1.7 g 7.55 mmol) in anhydrous tetrahydrofuran (15 mL) was added lithium aluminum hydride (1.0 M solution in THF, 7.5 mL, 7.55 mmol) at 0° C. over 30 minutes, and the reaction mixture was stirred at 0° C. for 90 minutes. The reaction mixture was quenched with water, and extracted with ethyl acetate (2×50 mL). The combined organic layers were dried using Na₂SO₄ and concentrated. The compound was purified by silica gel column chromatography eluting 15% EtOAc/hexanes to provide the title compound (1.2 g, 86.95%), ¹H NMR (400 MHz, DMSO-d₆) δ ppm 4.90-4.93 (t, 1H), 4.29-4.30 (d, 2H), 3.18-3.25, (m, 1H), 3.00 3.07 (m, 1H), 1.21-1.25 (m, 12H).

### (d) 4-(chloromethyl)-3,5-diisopropylisoxazole

This compound was synthesized from (3,5-diisopropylisoxazol-4-yl)methanol essentially as described in example 10(d). (1.0 g, 76.33%), ¹H NMR (400 MHz, DMSO-d₆) δ ppm 4.45 (s, 2H), 3.16-3.22, (m, 1H), 3.03 3.10 (m, 1H), 1.29-1.39 (m, 12H).

### (e) methyl 2-(4-((3,5-diisopropylisoxazol-4-yl)methoxy)phenyl)acetate

This compound was synthesized from methyl 2-(4-hydroxyphenyl)acetate and 4-(chloromethyl)-3,5-diisopropylisoxazole essentially as described in example 11(a). (0.300 g, 60.4%).

### (f) 2-(4-((3,5-diisopropylisoxazol-4-yl)methoxy)phenyl)acetic acid

This compound was synthesized from methyl 2-(4-((3,5-diisopropylisoxazol-4-yl)methoxy)phenyl)acetate essentially as described in example 1 (c). (0.215 g, 74.91%), ¹H NMR (400 MHz, DMSO-d₆) δ ppm 12.25 (br, 1H), 7.18-7.20 (d, 2H), 6.95-6.97 (d, 2H), 4.98 (s, 2H), 3.49 (s, 2H), 3.25-3.32 (m, 1H), 3.01-3.08 (m, 1H), 1.21-1.30 (m, 12H).

### (g) 2-(4-((3,5-diisopropylisoxazol-4-yl)methoxy)phenyl)-N-((2,4-dimethylphenyl)(phenyl)methyl)acetamide

This compound was synthesized from 2-(4-((3,5-diisopropylisoxazol-4-yl)methoxy)phenyl)acetic acid and (2,4-dimethylphenyl)(phenyl)methanamine essentially as described in example 3 (c). (56 mg, 35.00%). LCMS-X1: 511.4 [M+H]⁺; Rₜ=5.99 min. ¹H NMR (400 MHz, DMSO-d₆) δ ppm 8.83-8.85 (d, 1H), 7.29-733 (m, 2H), 7.15-7.26 (m, 5H), 6.92-6.99 (m, 5H), 6.17-6.19 (d, 1H), 4.88 (s, 2H), 3.35 (s, 2H), 3.24-3.33 (m, 1H), 3.00-3.07 (m, 1H), 2.15 (s, 3H), 2.20 (s, 3H), 1.20-1.24 (m, 12H).

### Example 20

### N-(4-((3,5-dimethylisoxazol-4-yl)methoxy)benzyl)-2-(2,4-dimethylphenyl)-N-methyl-2-phenylacetamide

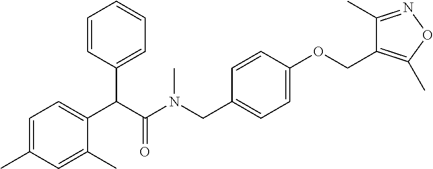 [SMILES: Cc1ccc(C(C(=O)N(C)Cc2ccc(OCc3c(C)noc3C)cc2)c2ccccc2)c(C)c1]

### (a) 2-(2,4-dimethylphenyl)-2-phenylacetic acid

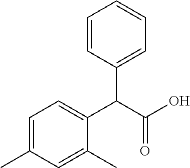 [SMILES: Cc1ccc(C(C(=O)O)c2ccccc2)c(C)c1]

The solution of mandelic acid (10.0 g, 65.7 mmol) in m-xylene (56.79 mL, 460 mmol) was heated to 60-70° C. followed by addition of SnCl₄ (11.5 mL, 98.6 mmol) over 2 h. The reaction mixture was then cooled to rt and stirred for 6 h at rt. The completion of the reaction was monitored by TLC on silica gel using hexanes:EtOAc (1:1). After completion of the reaction, iced-water (100 mL) was added into the reaction mixture, and the mixture was extracted with diethyl ether (3×250 mL). The combined organic layers were discarded. The remaining reaction mixture was extracted with 8% aqueous Na₂CO₃ (10×50 mL) and the combined aqueous layer was then acidified using 6 N HCl (20 mL) and the solid obtained was filtered and dried. The crude solid product was purified using silica gel column chromatography using 20% EtOAc/hexanes to obtain the title compound (6.25 g, 39.63%) as a solid. ¹H NMR (400 MHz, DMSO-d₆) δ ppm 12.6 (s, 1H), 7.21-7.33 (m, 6H), 7.09-7.12 (m, 1H), 6.97-7.06 (m, 3H), 5.12 (s, 1H), 2.23 (s, 3H), 2.17-2.18 (d, 4H).

### (b) 2-(2,4-dimethylphenyl)-N-(4-hydroxybenzyl)-2-phenylacetamide

To a solution of 2-(2,4-dimethylphenyl)-2-phenylacetic acid (0.25 g, 1.04 mmol) in anhydrous DMF (10 mL) at 25° C. was added EDC (0.237 g, 1.24 mmol) and the reaction mixture was stirred for 30 min at rt under argon atmosphere followed by addition of HOBt (0.19 g, 1.24 mmol), 4-dimethylamino pyridine (0.19 g, 1.56 mmol) and 4-(aminomethyl)phenol (0.154 g, 1.24 mmol), and the reaction mixture was stirred overnight under argon atmosphere. The raction mixture was cooled to 0° C. Water (10 mL) was added and the solid precipitate was filtered, washed with hexanes (2×10 mL), and dried under vacuum to provide the title compound (0.065 g, 18.10%) ¹H NMR (400 MHz, DMSO-d₆) δ ppm 9.29 (s, 1H), 8.55-8.58 (t, 1H), 7.26-7.30 (t, 2H), 7.16-7.20 (m, 4H), 7.00-7.02 (d, 2H), 6.93-6.95 (d, 2H), 6.65-6.68 (d, 2H), 5.07 (s, 1H), 4.11-4.22 (m, 2H), 2.22 (s, 3H), 2.15 (s, 3H).

### (c) N-(4-((3,5-dimethylisoxazol-4-yl)methoxy)benzyl)-2-(2,4-dimethylphenyl)-2-phenylacetamide

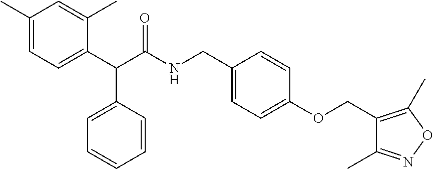 [SMILES: Cc1ccc(C(C(=O)NCc2ccc(OCc3c(C)noc3C)cc2)c2ccccc2)c(C)c1]

To a solution of 2-(2,4-dimethylphenyl)-N-(4-hydroxybenzyl)-2-phenylacetamide (0.06 g, 0.17 mmol) in acetonitrile (3 mL) was added cesium carbonate (0.17 g, 0.52 mmol) and 4-(chloromethyl)-3,5-dimethylisoxazole (0.03 g, 0.21 mmol). The resulting mixture was heated at 70° C. and stirred for 2 h in a sealed-tube. After completion of the reaction, water (10 mL) was added, and the mixture was extracted with ethyl acetate (2×15 mL). The combined organic layers were dried over Na₂SO₄ and evaporated under vacuum to obtain crude product which was purified by preparatory TLC on silica gel to provide the title compound (50.1 mg, 63.57%) ¹H NMR (400 MHz, DMSO-d₆) δ ppm 8.61-8.64 (s, 1H), 7.26-7.30 (t, 2H), 7.14-7.23 (m, 5H), 5.08 (s, 1H), 4.88 (s, 2H), 4.21-4.24 (t, 2H), 2.38 (s, 3H), 2.15-2.22 (t, 9H), (d)N-(4-((3,5-dimethylisoxazol-4-yl)methoxy)benzyl)-2-(2,4-dimethylphenyl)-N-methyl-2-phenylacetamide

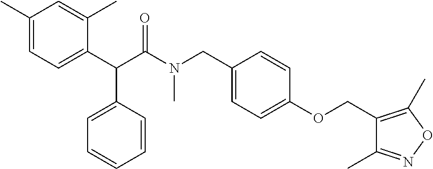 [SMILES: Cc1ccc(C(C(=O)N(C)Cc2ccc(OCc3c(C)noc3C)cc2)c2ccccc2)c(C)c1]

To a solution of 60% NaH (0.01 g, 0.20 mmol) in anhydrous THF (2 mL) at 25° C. was added N-(4-((3,5-dimethylisoxazol-4-yl)methoxy)benzyl)-2-(2,4-dimethylphenyl)-2-phenylacetamide (0.075 g, 0.24 mmol) in THF (2 mL) dropwise and the reaction was stirred for 30 min at rt under argon atmosphere followed by addition of iodomethane (0.052 g, 0.24 mmol) dropwise and the reaction was stirred for 2 h under argon atmosphere. After completion of the reaction, water (10 mL) was added and the mixture was extracted with ethyl acetate (2×15 mL). The combined organic layers were washed with brine (10 mL), dried over Na₂SO₄, and evaporated under reduced pressure to obtain crude product which was purified by preparatory TLC on silica gel to provide the title compound (0.0509 g, 54.320/o). LCMS-G30: 466.6 [M+H]⁺; Rₜ=7.25 min. ¹H NMR (400 MHz, DMSO-d₆) δ ppm 7.27-7.32 (m, 3H), 7.19-7.24 (m, 1H), 7.16-7.18 (m, 3H), 6.94-7.08 (m, 6H), 6.85-6.87 (d, 1H), 5.43 (s, 1H), 4.89 (s, 2H), 4.54-4.57 (d, 1H), 4.37-4.41 (d, 1H), 3.3 (s, 3H), 2.67 (s, 3H), 2.46 (s, 3H), 2.33 (s, 3H), 2.20 (s, 3H), 2.15 (s, 3H).

### Example 21

### N-((4-chlorophenyl)(phenyl)methyl)-2-(5-((3,5-dimethylisoxazol-4-yl)methoxy)pyridin-2-yl)acetamide

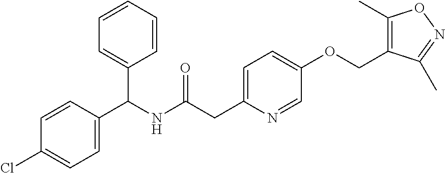 [SMILES: Cc1noc(C)c1COc1ccc(CC(=O)NC(c2ccccc2)c2ccc(Cl)cc2)nc1]

### (a) methyl 5-hydroxypicolinate

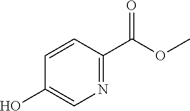

To a solution of 5-hydroxypicolinic acid (4.0 g, 28.77 mmol) in methanol (75 mL), cone. H₂SO₄ (5 mL) was added dropwise at 0° C. and the reaction mixture was refluxed at 80° C. for 6 h. Methanol was removed under reduced pressure. The residue was dissolved in water and neutralized by sodium bicarbonate solution. Product was extracted with (3×50 mL) ethyl acetate. The combined organic layers were dried over Na₂SO₄ and concentrated to provide the title compound (2.10 g, 47.72%). MS (ESI+) 154.08 (M+H). ¹H NMR (400 MHz, DMSO-d₆) δ ppm 10.86 (s, 1H), 8.20 (s, 1H), 7.93-7.98 (d, 1H), 7.25-7.28 (s, 1H), 3.81 (s, 3H).

### (b) methyl 5-((3,5-dimethylisoxazol-4-yl)methoxy)picolinate

To a solution of methyl 5-hydroxypicolinate (3.0 g, 19.60 mmol) in dimethylformamide (35 mL) was added potassium carbonate (4.05 g, 29.41 mmol) and the reaction was stirred at rt for 30 min. 4-(chloromethyl)-3,5-dimethylisoxazole (3.41 g, 23.52 mmol) was added and the reaction mixture was stirred at 80° C. for 6 h. After completion of the reaction, the reaction mixture was quenched by addition of water (50 mL) and the mixture was extracted with 2×50 mL ethyl acetate. The organic layers were dried (Na₂SO₄) and concentrated to obtain a crude product which was purified by silica gel column chromatography (30% EtOAc/hexanes) to provide the title compound (3.9 g, 76.47%). MS (ESI+) 263.00 (M+H). ¹H NMR (400 MHz, DMSO-d₆) δ ppm 8.43 (s, 1H), 8.07-8.09 (d, 1H), 7.60-7.63 (d, 1H), 5.11 (s, 2H), 3.85 (s, 3H), 2.43 (s, 3H), 2.23 (s, 3H).

### (c) (5-((3,5-dimethylisoxazol-4-yl)methoxy)pyridin-2-yl)methanol

To a solution of methyl 5-((3,5-dimethylisoxazol-4-yl)methoxy) picolinate (3.80 g, 14.50 mmol) in methanol (150 mL), sodium borohydride (10.74 g, 290.07 mmol) was added portion wise at 0° C. and stirred at rt for 3 h. After completion of the reaction, methanol was evaporated and diluted with water. The product was extracted with 2×100 mL ethyl acetate. The combined organic layers were dried over Na₂SO₄ and concentrated to obtain the title compound (3.1 g, 91.44%). ¹H NMR (400 MHz, DMSO-d₆) δ ppm 8.23 (s, 1H), 7.46-7.48 (s, 1H), 7.38-7.40 (s, 1H), 5.31-5.34 (t, 1H), 4.99 (s, 2H), 4.48 (s, 2H), 2.40 (s 3H), 2.21 (s, 3H).

### (d) 4-(((6-(chloromethyl)pyridin-3-yl)oxy)methyl)-3,5-dimethylisoxazole

This compound was synthesized from (5-((3,5-dimethylisoxazol-4-yl)methoxy)pyridin-2-yl)methanol essentially as described in example 10(d). (3.5 g, 94.59%). MS (ESI+) 253.00 [M+H]. ¹H NMR (400 MHz, DMSO-d₆) δ ppm 8.40 (s, 1H), 7.61-7.67 (m, 2H), 5.06 (s, 2H), 4.81 (s, 2H), 2.42 (s, 3H), 2.22 (s, 3H).

### (e) 2-(5-((3,5-dimethylisoxazol-4-yl)methoxy)pyridin-2-yl)acetonitrile

To a solution of 4-(((6-(chloromethyl)pyridin-3-yl)oxy)methyl)-3,5-dimethylisoxazole (1.0 g, 3.968 mmol) in methanol (35 mL) and water mixture was added potassium iodide (0.05 g, 0.30 mmol) and sodium cyanide (0.388 g, 7.936 mmol). Reaction was refluxed at 80° C. for 4 h. After completion of the reaction, the methanol was evaporated. The reaction mixture was quenched by addition of water (50 mL) and extracted it with ethyl acetate (2×25 mL). The combined organic layers were dried over Na₂SO₄ and concentrated to provide the title compound which was used as crude in the next step, (0.90 g, 93.75%). MS (ESI+) 244.10 (M+H),

### (f) 2-(5-((3,5-dimethylisoxazol-4-yl)methoxy)pyridin-2-yl)acetic acid

To a solution of 2-(5-((3,5-dimethylisoxazol-4-yl)methoxy)pyridin-2-yl)acetonitrile (0.90 g, 3.70 mmol) in methanol (35 mL), sodium hydroxide (0.444 g, 11.1 mmol) in water (50 mL) was added dropwise and the reaction was refluxed at 80° C. for 6 h. After completion of the reaction, the methanol was distilled off at reduced pressure and water (50 mL) was added to the reaction mixture. The aqueous layer was washed with ethyl acetate and then the aqueous layer was acidified to pH=3 using 10% Hydrochloric acid and extracted with ethyl acetate (2×50 mL). The combined organic layers were dried over Na₂SO₄ and concentrated to provide the title compound (0.160 g, 16.49%) MS (ESI+) 263.1 (M+H).

### (g) N-((4-chlorophenyl)(phenyl)methyl)-2-(5-((3,5-dimethylisoxazol-4-yl)methoxy)pyridin-2-yl)acetamide

To a solution of 2-(5-((3,5-dimethylisoxazol-4-yl)methoxy)pyridin-2-yl)acetic acid (0.150 g 0.572 mmol) in tetrahydrofuran (25 mL), EDC (0.164 g, 0.858 mmol) was added portion wise and the reaction was stirred at rt for 2 h. (4-chlorophenyl) (phenyl) methanamine hydrochloride (0.200 g, 0.858 mmol), HOBt (0.100 g, 0.572 mmol), and triethylamine (0.237 mL, 1.751 mmol) were then added and the reaction mixture was stirred for 24 h at rt. After completion of the reaction, water (50 mL) was added to the reaction mixture and extracted with ethyl acetate (2×50 mL). The combined organic layers were dried over Na₂SO₄ and concentrated to obtain a crude product which was purified by silica gel column chromatography using (30% EtOAc/hexanes) as mobile phase to provide the title compound (0.130 g, 49.24%). MS (ESI+) 462.34 [M+H]. ¹H NMR (400 MHz, DMSO-d₆) δ ppm 9.05 (d, 1H), 8.24 (s, 1H), 7.33-7.42 (m, 3H), 7.24-7.32 (m, 7H), 6.10-6.12 (d, 1H), 4.98 (s, 2H), 3.69 (s, 2H), 2.40 (s, 3H), 2.18 (s, 3H).

### Example 22

### N-((4-chlorophenyl)(phenyl)methyl)-2-(4-((3,5-dimethylisoxazol-4-yl)methylthio)phenyl)acetamide

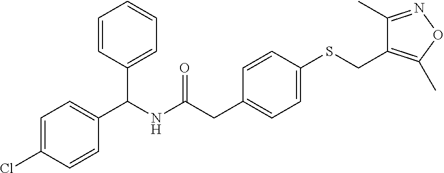 [SMILES: Cc1noc(C)c1CSc1ccc(CC(=O)NC(c2ccccc2)c2ccc(Cl)cc2)cc1]

### (a) 2-(4-((3,5-dimethylisoxazol-4-yl)methylthio)phenyl)acetic acid

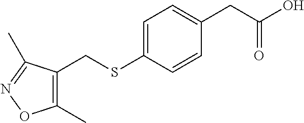 [SMILES: Cc1noc(C)c1CSc1ccc(CC(=O)O)cc1]

To a solution of 2-(4-mercaptophenyl)acetic acid (0.25 g, 1.40 mmol) in DMF (3 mL) at 25° C. was added 4-(chlorophenyl)-3,5-dimethylisoxazole (0.227 g, 1.5 mmol) followed by addition of potassium hydroxide (0.208 g, 3.7 mmol) in two drops of water. After completion of the addition, the reaction mixture was heated to 120° C. for 2 h. After completion of the reaction, the reaction mixture was cooled to rt, water (10 mL) was added and the mixture was acidified to pH=2 using 10% aqueous HCl. The aqueous layer then was extracted with ethyl acetate (2×25 mL). The combine organic layers were washed with brine (25 mL), dried over (Na₂SO₄), and evaporated to obtain the title compound as a light yellow oil (0.46 g) which was used in the next step without further purification. ¹H NMR (400 MHz, DMSO-d₆) δ ppm 12.45 (br, 1H), 7.19-7.29 (m, 4H), 3.93 (s, 2H), 3.55 (s, 2H), 2.14 (s, 3H), 2.04 (s, 3H).

### (b) N-((4-chlorophenyl)(phenyl)methyl)-2-(4-((3,5-dimethylisoxazol-4-yl)methylthio)phenyl)acetamide

To a solution of 2-(4-((3,5-dimethylisoxazol-4-yl)methylthio)phenyl)acetic acid (0.460 g, 1.6 mmol) in DMF (5 mL) at 25° C. was added HOBt (0.304 g, 1.8 mmol), EDC (0.382 g, 1.9 mmol), and DMAP (0.405 g, 3.2 mmol) and the resulting mixture was stirred at rt for 10 minutes. (4-chlorophenyl)(phenyl)methanamine hydrochloride (0.466 g, 1.8 mmol) in DMF (1 mL) was added slowly and the stirring at rt was continued overnight. After completion of the reaction, water (10 mL) was added into the reaction mixture and extracted with ethyl acetate (2×25 mL). The combined organic layers were washed with brine (10 mL), dried over (Na₂SO₄), and evaporated to obtain a crude product which was purified using silica gel column chromatography using 24% ethyl acetate:hexanes and then purified using prep TLC on silica gel (50% ethyl acetate in hexanes+5 drops of acetic acid) to obtain the title compound (66 mg, 16.68%). LCMS-X1: 477.2 [M+H]⁺; Rₜ=3.61 min. ¹H NMR (400 MHz, CDCl₃) δ ppm 7.28-7.35 (m, 6H), 7.21-7.23 (d, 2H), 7.06-7.11 (m, 4H), 6.21-6.23 (d, 1H), 5.93-5.94 (d, 1H), 3.76 (s, 2H), 3.63 (s, 2H), 2.24 (s, 3H), 2.01 (s, 3H).

### Example 23

### N-((4-chloro-2-methylphenyl)(phenyl)methyl)-2-(4-(2-(3,5-dimethylisoxazol-4-yl)ethyl)piperazin-1-yl)acetamide

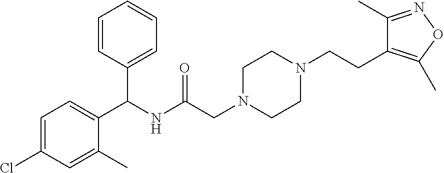 [SMILES: Cc1cc(Cl)ccc1C(NC(=O)CN1CCN(CCc2c(C)noc2C)CC1)c1ccccc1]

### (a) tert-butyl 4-(2-(3,5-dimethylisoxazol-4-yl)ethyl)piperazine-1-carboxylate

To the solution of 4-(2-chloroethyl)-3,5-dimethylisoxazole (159 mg, 1 mmol) in MeCN (5 mL) was added tert-butyl piperazine-1-carboxylate (205 mg, 1.1 mmol), then potassium bicarbonate (200 mg, 2 mmol) and potassium iodide (34 mg, 0.2 mmol). Then the resulting mixture was heated to reflux overnight. After cooling to rt, the mixture was extracted with EtOAc. The combined organic layers were washed with brine and dried over Na₂SO₄. After removal of solvent under reduced pressure, the residue was purified by silica gel column chromotography with EtOAc:petroleum ether=2:1 to afford the title compound as a yellow solid (189 mg, 61%). LCMS-P1: 310, [m+H]⁺.

### (b) 3,5-dimethyl-4-(2-(piperazin-1-yl)ethyl)isoxazole hydrochloride

To the solution of tert-butyl 4-(2-(3,5-dimethylisoxazol-4-yl)ethyl)piperazine-1-carboxylate (189 mg, 1 mmol) in MeOH (5 mL) was added HCl/Et₂O (1M, 2.5 mL, 2.5 mmol), then the mixture was stirred at rt overnight. The reaction mixture was evaporated under reduced pressure, the crude the title compound (140 mg, yield 97.5%) was used directly to the next step without further purification. LCMS-P1: 210 [M+H]⁺;

### (c) methyl 2-(4-(2-(3,5-dimethylisoxazol-4-yl)ethyl)piperazin-1-yl)acetate

To the mixture of 3,5-dimethyl-4-(2-(piperazin-1-yl)ethyl)isoxazole hydrochloride (140 mg, 0.57 mmol) in DMF (3 mL) was added K₂CO₃ (236 mg, 1.71 mmol) and methyl 2-bromoacetate (105 mg, 0.68 mmol), then the mixture was heated at 60° C. overnight under argon. The reaction mixture was cooled to rt and poured into water. The mixture was extracted with EtOAc. The combined organic layers were washed with brine and dried over Na₂SO₄. After evaporation of solvent under reduced pressure, the residue was purified by flash chromotography to give the title compound as light-yellow oil (97 mg, yield 60%). LCMS-P1: 282 [M+H]⁺;

### (d) 2-(4-(2-(3,5-dimethylisoxazol-4-yl)ethyl)piperazin-1-yl)acetic acid

To the suspension of methyl 2-(4-(2-(3,5-dimethylisoxazol-4-yl)ethyl)piperazin-1-yl)acetate (97 mg, 0.345 mmol) in THF/Water (4 mL, 1:1) was added LiOH (29 mg, 0.69 mmol), and the resulting mixture was stirred at rt for 30 min. The reaction mixture was neutralized by HCl (1M). After removal of all the solvent, the crude the title compound was used directly to the next step without further purification (110 mg). LCMS-P1: 268 [M+H]⁺

### (e) N-((4-chloro-2-methylphenyl)(phenyl)methyl)-2-(4-(2-(3,5-dimethylisoxazol-4-yl)ethyl)piperazin-1-yl)acetamide

To the solution of 2-(4-(2-(3,5-dimethylisoxazol-4-yl)ethyl)piperazin-1-yl)acetic acid (110 mg, 0.345 mmol) in DMF (3 mL) was added (4-chloro-2-methylphenyl)(phenyl)methanamine (111 mg, 0.414 mmol), EDC (79 mg, 0.414 mmol), HOBt (56 mg, 0.414 mmol), then DIPEA (89 mg, 0.69 mmol). After stirring at 45° C. overnight, the mixture was poured into water, and extracted with EtOAc (15 mL×2). The combined organic layers were washed with brine and dried over Na₂SO₄. After removal of solvent, the residue was purified by preparatory HPLC using 10-100% water/acetonitrile with 0.1% TFA to afford the title compound as a white solid (20 mg, yield 12%). LCMS-P1: 481[M+H]⁺. ¹H NMR (400 MHz, DMSO-d₆) δ ppm 8.48 (d, J=8 Hz, 1H), 7.34 (t, J=0.76 Hz, 2H), 7.29-7.25 (m, 3H), 7.20-7.15 (m, 3H), 6.23 (d, J=8 Hz, 1H), 3.02 (s, 2H), 2.43-2.30 (m 12H), 2.27 (s, 3H), 2.23 (s, 3H), 2.14 (s 3H).

### Example 24

### methyl 2-((3,5-dimethylisoxazol-4-yl)methoxy)-5-(2-(((2,4-dimethylphenyl)(phenyl)methyl)amino)-2-oxoethyl)benzoate

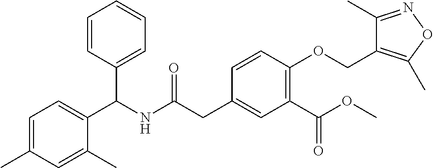 [SMILES: COC(=O)c1cc(CC(=O)NC(c2ccccc2)c2ccc(C)cc2C)ccc1OCc1c(C)noc1C]

### (a) benzyl 2-(4-hydroxyphenyl)acetate

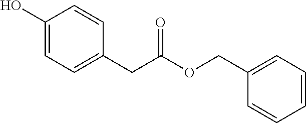 [SMILES: O=C(Cc1ccc(O)cc1)OCc1ccccc1]

Sodium hydride was suspended in 100 ml of DMF and 2-(4-hydroxyphenyl)acetic acid (9 g, 39.4 mmol) was added potionwise to the stirred mixture at 0° C. After 30 minutes, benzyl bromide (30 ml) was added dropwise in 1 h. The mixture was diluted with water (100 mL) and extracted with EtOAc (100 mL×3). The combined organic solvents were dried over Na₂SO₄. After removal of solvent, the crude compound was purified by column chromatography on silica gel (EtOAc:petroleum ether=1:5) to provide the title compound (8.7 g, 91.2%). LCMS-P1: 243.0 [M+H]⁺; Rₜ=1.85 min,

### (b) benzyl 2-(3-formyl-4-hydroxyphenyl)acetate

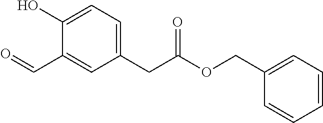 [SMILES: O=Cc1cc(CC(=O)OCc2ccccc2)ccc1O]

To a solution of benzyl 2-(4-hydroxyphenyl)acetate (4.84 g, 20 mmol) in CH₃CN (150 mL) was added MgCl₂ (3.8 g, 40 mmol) and triethylamine (8.08 g, 80 mmol) under nitrogen and the mixture was refluxed for 1 h. Then (CH₂O) was added and the reaction was refluxed overnight. After cooling to rt, ether (200 mL) and 1 M HCl (100 mL) were added. The organic layer was separated and washed with 1M HCl (100 mL×3), and dried over Na₂SO₄. After removal of solvent, the residue was purified by silica gel column chromotography (petroleum ether/EtOAc=5/1) to obtain the title compound (2.71 g, 50%). LCMS-P1: 271.1 [M+H]⁺; Rt: 1.93 min

### (c) benzyl 2-(4-((3,5-dimethylisoxazol-4-yl)methoxy)-3-formylphenyl)acetate

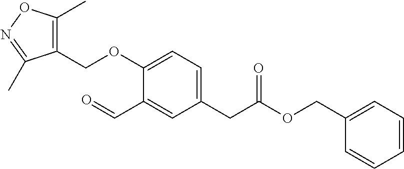 [SMILES: Cc1noc(C)c1COc1ccc(CC(=O)OCc2ccccc2)cc1C=O]

To a solution of benzyl 2-(3-formyl-4-hydroxyphenyl)acetate (1.35 g, 10 mmol) in MeCN (50 mL) were added 4-(chloromethyl)-3,5-dimethylisoxazole (0.73 g, 10 mmol), K₂CO₃ (0.74 g, 15 mmol) and potassium iodide (0.41 g, 5 mmol). The reaction mixture was refluxed overnight. After the completion of the reaction, the mixture was diluted with water (30 mL) and extracted with EtOAc (20 mL×3). The combined organic layers were dried over Na₂SO₄. After removal of the solvent, the crude product was purified by column chromatography on silica gel (EtOAc:petroleum ether=2:1) to provide the title compound (1.5 g, 41%). LCMS-P: 380.0 [M+H]⁺; Rₜ=1.72 min,

### (d) 5-(2-(benzyloxy)-2-oxoethyl)-2-((3,5-dimethylisoxazol-4-yl)methoxy)benzoic acid

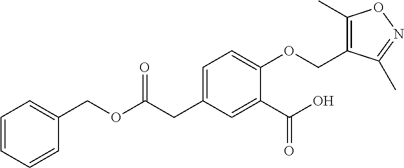 [SMILES: Cc1noc(C)c1COc1ccc(CC(=O)OCc2ccccc2)cc1C(=O)O]

To a solution of benzyl 2-(4-((3,5-dimethylisoxazol-4-yl)methoxy)-3-formylphenyl)acetate (0.6 g, 1.56 mmol) in DMSO (15 mL) were added sodium dihydrogen phosphate (0.48 g, 3.96 mmol, dissolved in 4 mL water) and Sodium chlorite (0.29 g, 3.12 mmol in 8 ml water). The reaction mixture was stirred at rt overnight. Then sat. aq. Na₂CO₃ (20 ml) was added in 5 min. The mixture was diluted with water (30 mL) and extracted with EtOAc (20 mL×3). The combined organic solvents were dried over Na₂SO₄. After removal of solvent, the crude compound was purified by column chromatography on silica gel (EtOAc:petroleum ether=1:1) to provide the title compound (430 mg, 69.8%). LCMS-P1: 396.1 [M+H]⁺; Rₜ=1.57 min

### (e) methyl 5-(2-(benzyloxy)-2-oxoethyl)-2-((3,5-dimethylisoxazol-4-yl)methoxy)benzoate

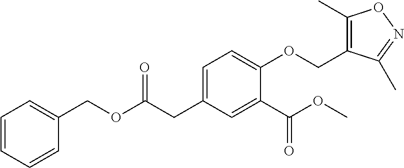 [SMILES: COC(=O)c1cc(CC(=O)OCc2ccccc2)ccc1OCc1c(C)noc1C]

To a solution of 5-(2-(benzyloxy)-2-oxoethyl)-2-((3,5-dimethylisoxazol-4-yl)methoxy)benzoic acid (0.2 g, 0.51 mmol) in DMF (15 mL) were added iodomethane (72 mg, 0.51 mmol) and K₂CO (140 mg, 1.02 mmol). The reaction mixture was stirred at rt overnight. Then sat. aq. Na₂CO₃ (20 ml) was added over 5 min. The mixture was diluted with water (30 mL) and extracted with EtOAc (20 mL×3). The combined organic solvents were dried over Na₂SO₄. After removal of solvent, the crude compound was purified by column chromatography on silica gel (EtOAc:petroleum ether=1:3) to provide the title compound (160 mg, 76.7%). LCMS-P1: 410.1 [M+H]⁺; Rₜ=1.99 min

### (f) 2-(4-((3,5-dimethylisoxazol-4-yl)methoxy)-3-(methoxycarbonyl)phenyl)acetic acid

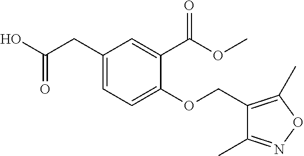 [SMILES: COC(=O)c1cc(CC(=O)O)ccc1OCc1c(C)noc1C]

To a solution of methyl 5-(2-(benzyloxy)-2-oxoethyl)-2-((3,5-dimethylisoxazol-4-yl)methoxy)benzoate (60 mg, 0.15 mmol) in EtOH (3 mL) was added 10% Pd/C (12 mg). The reaction mixture was stirred under H₂ atmosphere at rt for 1.5 h. After the completion of the reaction, the reaction mixture was diluted with water (30 mL) and extracted with EtOAc (20 mL×3). The combined organic solvents were dried over Na₂SO₄. After removal of solvent, the crude product was purified by column chromatography on silica gel (EtOAc:petroleum ether=1:5) to provide the title compound (40 mg, 83.6%). LCMS-P1: 342.1 [M+Na]⁺; Rₜ=1.42 min.

### (g) methyl 2-((3,5-dimethylisoxazol-4-yl)methoxy)-5-(2-(((2,4-dimethylphenyl)(phenyl)methyl)amino)-2-oxoethyl)benzoate

This compound was synthesized from 2-(4-((3,5-dimethylisoxazol-4-yl)methoxy)-3-(methoxycarbonyl)phenyl)acetic acid and (2,4-dimethylphenyl)(phenyl)methanamine essentially as described in example 4 (b) and was purified by column chromatography on silica gel (EtOAc:petroleum ether=1:3) to provide the title compound (25 mg, 40.7%). LCMS-P1: 535.0 [M+Na]+; Rₜ=1.83 min. ¹H NMR (400 MHz, DMSO-d₆) δ ppm 7.65 (d, J=2.0 Hz, 1H), 7.36 (dd, J=2.4 Hz, 1H), 7.20 (s, 2H), 7.18 (s, 1H), 7.03 (d. J=6.8 Hz, 2H), 6.93 (t, J=6.0 Hz, 2H), 6.87 (d, J=8.0 Hz, 1H), 6.76 (d, J=8.0 Hz, 1H), 6.30 (d, J=8.0 Hz, 1H), 5.85 (d, J=8.0 Hz, 1H), 4.80 (s, 2H), 3.77 (3, 3H), 3.51 (s, 2H), 2.32 (s, 3H), 2.25 (is, 3H), 2.22 (s, 3H), 2.15 (s, 3H).

### Example 25

### 2-(4-(2-(3,5-dimethylisoxazol-4-yl)-2-hydroxyethoxy)phenyl)-N-((2,4-dimethylphenyl)(phenyl)methyl)acetamide

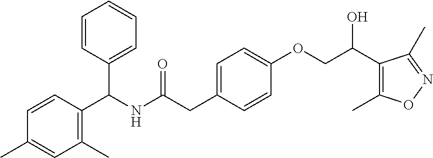 [SMILES: Cc1ccc(C(NC(=O)Cc2ccc(OCC(O)c3c(C)noc3C)cc2)c2ccccc2)c(C)c1]

### (a) methyl 3,5-dimethylisoxazole-4-carboxylate

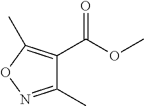

To a solution of 3,5-dimethylisoxazole-4-carboxylic acid (9.2 g, 65.2 mmol) in MeOH (50 mL) was added SOCl₂ (15.3 g, 130.4 mmol) very slowly. The reaction mixture was heated to 70° C. overnight. The reaction mixture was then cooled to rt, concentrated, and purified by column chromatography (10% EtOAc/petroleum ether) to afford the title compound (9.0 g, 89%). LCMS-P1: 156 [M+H]*; Rₜ: 1.404 min.

### (b) (3,5-dimethylisoxazol-4-yl)methanol

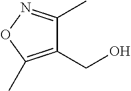

To a stirred solution of methyl 3,5-dimethylisoxazole-4-carboxylate (9.0 g, 58 mmol) in THF (200 mL) at 0° C. was added LiAlH₄ (2.42 g, 63.8 mmol) portionwise. After addition, the reaction mixture was allowed to warm up to rt and stirred overnight. The reaction mixture was quenched by the successive addition of water (2.5 mL), 10% aq. NaOH (5 mL), and water (7.5 mL). The organic layer was separated, dried over Na₂SO₄, filtered, and concentrated to afford the title compound (5.0 g, 68%). LCMS-P1: 128 [M+H]⁺; Rₜ: 0.963 min. ¹H NMR (400 MHz, CDCl₃) δ ppm 4.37 (s, 2H), 2.30 (s, 3H), 2.20 (s, 3H).

### (c) 3,5-dimethylisoxazole-4-carbaldehyde

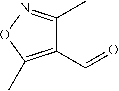

To a solution of (3,5-dimethylisoxazol-4-yl)methanol (1.00 g, 7.86 mmol) in CH₂C2 (20 mL) at 0° C. was added Dess-Martin periodinane (4.17 g, 9.83 mmol) over 10 min and the reaction mixture was warmed to rt. The reaction mixture was stirred at rt for 60 min and then filtered through Celite® and washed through with CH₂Cl₂. The organic layer was dried over Na₂SO₄, concentrated, and purified by column chromatography (15% EtOAc/hexanes) to provide the title compound (0.450 g, 46%). ¹H NMR (400 MHz, DMSO-d₆) δ ppm 9.92 (s, 1H), 2.68 (s, 3H), 2.37 (s, 3H).

### (d) 1-(3,5-dimethylisoxazol-4-yl)ethanol

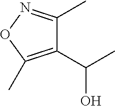

To a solution of 3,5-dimethylisoxazole-4-carbaldehyde (0.450 g, 0.36 mmol) in dry THF (3 mL) at 0° C. was added MeMgBr (3.0 M solution in Et₂O) (1.2 mL, 3.60 mmol) dropwise over 10 min and the reaction mixture was warmed to rt. The reaction mixture was stirred at rt for 90 min. Water (10 mL) was added very slowly and the reaction mixture was extracted with EtOAc (100 mL). The organic layer was dried over Na₂SO₄, concentrated, and purified by column chromatography (27% EtOAc/hexanes) to provide the title compound (0.480 g, 95%). ¹H NMR (400 MHz, DMSO-d₆) δ ppm 5.07 (d, 1H), 4.65-4.70 (m, 1H), 2.33 (s, 3H), 2.20 (s, 3H), 1.32 (d, 3H).

### (e) 1-(3,5-dimethylisoxazol-4-yl)ethanone

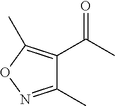

To a solution of 1-(3,5-dimethylisoxazol-4-yl)ethanol (0.30 g 2.12 mmol) in CH₂Cl₂ (8 mL) at 0° C. was added PCC (0.068 g, 3.19 mmol) and reaction was stirred for 1 h at rt. Water (25 mL) was added slowly and the reaction mixture was extracted with CH₂Cl₂ (3×10 mL). The organic layer was dried over Na₂SO₄, filtered, concentrated, and purified by column chromatography (10%/0 EtOAc/hexane) to provide the title compound (0.10 g, 34%). ¹H NMR (400 MHz, CDCl₃) δ ppm 2.68 (s, 3H), 2.44 (s, 5H), 2.24 (s, 1H).

### (f) 2-bromo-1-(3,5-dimethylisoxazol-4-yl)ethanone

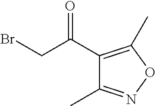

To a solution of 1-(3,5-dimethylisoxazol-4-yl)ethanone (1.0 g, 7.13 mmol) in CCl₄ (40 mL) was added AcOH (1.0 g, 1.142 mmol). The reaction mixture was stirred at 48° C., followed by dropwise addition of Br₂ (0.37 mL, 7.19 mmol) in CCl₄ (30 mL). The reaction mixture was stirred at 48° C. for an additional 20 min. Ice cold water (50 mL) was added and the reaction mixture was extracted with CH₂Cl₂ (3×25 mL). The organic layer was dried over Na₂SO₄, filtered, concentrated, and purified by column chromatography (5% EtOAc/hexane) to provide the title compound (0.80 g, 51%). ¹H NMR (400 MHz, CDCl₃) δ ppm 4.18 (s, 2H), 2.74 (s, 3H), 2.52 (s, 3H).

### (g) methyl 2-(4-(2-(3,5-dimethylisoxazol-4-yl)-2-oxoethoxy)phenyl)acetate

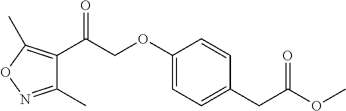 [SMILES: COC(=O)Cc1ccc(OCC(=O)c2c(C)noc2C)cc1]

A mixture of 2-bromo-1-(3,5-dimethylisoxazol-4-yl)ethanone (193 mg, 0.89 mmol), methyl 2-(4-hydroxyphenyl)acetate (147 mg, 0.89 mmol) and potassium carbonate (246 mg, 1.78 mol) in acetonitrile (10 mL) was stirred at rt for 1.5 h. After filtration, the filtrate was concentrated to obtain the title compound (270 mg, 99%) as a yellow oil. LCMS-P1: 304.1 [M+H]⁺; Rₜ=1.82 min.

### (h) methyl 2-(4-(2-(3,5-dimethylisoxazol-4-yl)-2-hydroxyethoxy)phenyl)acetate

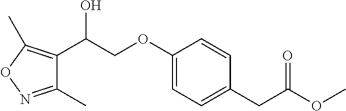 [SMILES: COC(=O)Cc1ccc(OCC(O)c2c(C)noc2C)cc1]

To a cooled (0° C.) solution of methyl 2-(4-(2-(3,5-dimethylisoxazol-4-yl)-2-oxoethoxy)phenyl)acetate (120 mg, 0.4 mmol) in MeOH (10 mL) was added NaBH₄ (76 mg, 2 mmol). After the addition, the reaction mixture was stirred at rt for 1 h. Then solvent was removed and water (10 mL) was added to the mixture. The mixture was extracted with EtOAc (15 ml×3). The organic layers were dried over anhydrous Na₂SO₄ and filtered. The solvent was removed to obtain the title compound (%96 mg, 79%) as colorless oil. The title compound was used in the next step without any further purification. LCMS-P1: 306.1 [M+H]⁺; Rₜ=1.73 min.

### (i) 2-(4-(2-(3,5-dimethylisoxazol-4-yl)-2-hydroxyethoxy)phenyl)acetic acid

This compound was prepared from methyl 2-(4-(2-(3,5-dimethylisoxazol-4-yl)-2-hydroxyethoxy)phenyl)acetate essentially as described in example 24 (d) (99 mg, 100%). LCMS-P1: 292.0 [M+H]⁺; Rₜ=1.32 min.

### (j) 2-(4-(2-(3,5-dimethylisoxazol-4-yl)-2-hydroxyethoxy)phenyl)-N-((2,4-dimethylphenyl)(phenyl)methyl)acetamide

This compound was prepared from 2-(4-(2-(3,5-dimethylisoxazol-4-yl)-2-hydroxyethoxy)phenyl)acetic acid and (2,4-dimethylphenyl)(phenyl)methanamine essentially as described in example 4 (b) (46 mg, 56%) as a white solid. LCMS-P1: 485.0 [M+H]⁺; Rₜ=1.75 min. ¹H NMR (400 MHz, CDCl₃) δ ppm 7.23-7.16 (m, J=7.6 Hz, 5H), 7.08 (d, J=6.4 Hz, 2H), 6.99 (s, 1H), 6.93-6.86 (m, 3H), 6.77 (d, J=8.0 Hz, 1H), 6.37 (d, J=8.0 Hz, 1H), 5.89 (d, J=8.4 Hz, 1H), 5.03-5.06 (m, 1H), 4.11-4.06 (m, 1H), 3.99 (dd, J=9.6 Hz, 3.6 Hz, 1H), 3.59 (s, 2H), 2.46 (s, 3H), 2.34 (s, 3H), 2.29 (s, 3H), 2.22 (s, 3H).

### Example 26

### 2-amino-2-(4-((3,5-dimethylisoxazol-4-yl)methoxy)phenyl)-N-((2,4-dimethylphenyl)(phenyl)methyl)acetamide

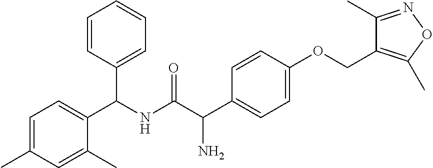 [SMILES: Cc1ccc(C(NC(=O)C(N)c2ccc(OCc3c(C)noc3C)cc2)c2ccccc2)c(C)c1]

### (a) tert-butyl (1-(4-((3,5-dimethylisoxazol-4-yl)methoxy)phenyl)-2-(((2,4-dimethylphenyl)(phenyl)methyl)amino)-2-oxoethyl)carbamate

This compound was synthesized essentially as described in example 1 starting from methyl 2-((tert-butoxycarbonyl)amino)-2-(4-hydroxyphenyl)acetate.

### (b) 2-amino-2-(4-((3,5-dimethylisoxazol-4-yl)methoxy)phenyl)-N-((2,4-dimethylphenyl)(phenyl)methyl)acetamide

tert-Butyl (1-(4-((3,5-dimethylisoxazol-4-yl)methoxy)phenyl)-2-(((2,4-dimethylphenyl)(phenyl)methyl)amino)-2-oxoethyl)carbamate (85 mg, 0.16 mmol) in dichloromethane (2 mL) and trifluorocetic acid (1 mL) were stirred for 15 minutes. Solvent was removed and product was purified by reversed phase HPLC column chromotography using 10-90% acetonitrile/water with 0.05% TFA to give the title compound (130 mg, 94%). LCMS-T1: 470.2 [M+H]+; Rₜ: 5.75 min. ¹H NMR (400 MHz, CDCl₃) δ ppm 7.34-7.19 (m, 5H), 7.10 (m, 1H), 6.97 (m, 1H), 6.93-6.87 (m, 2H), 6.81 (m, 1H), 6.60 (m, 1H), 6.19 (m, 1H), 5.10 (d, 1H), 4.78 (d, 2H), 2.39 (s, 3H), 2.27 (s, 3H), 2.25 (s, 3H), 1.98 (s, 3H).

Using essentially the same procedure as described in example 2, the following compounds in table 1 were made.

TABLE 1

| Ex. | Structure/Name | NMR | LCMS |
|---|---|---|---|
| 27 | 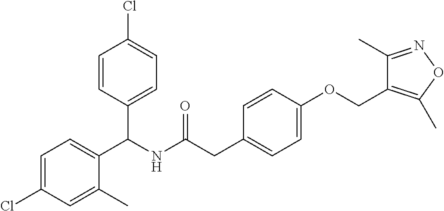 [SMILES: Cc1cc(Cl)ccc1C(NC(=O)Cc1ccc(OCc2c(C)noc2C)cc1)c1ccc(Cl)cc1] N-((4-chloro-2-methylphenyl)(4- chlorophenyl)methyl)-2-(4-((3,5- dimethylisoxazol-4- yl)methoxy)phenyl)acetamide | ¹H NMR (400 MHz, DMSO-d₆) δ ppm 8.91-8.93 (d, 1 H), 7.39-7.41 (d, 2 H), 7.28 (s, 1 H), 7.25 (s, 1 H), 7.17-7.23 (m, 4 H), 7.04-7.06 (d, 1 H), 6.92-6.94 (d, 2 H), 6.16- 6.18 (d, 1 H), 4.88 (s, 2 H), 3.44 (s, 2 H), 2.38 (s, 3 H), 2.18-2.20 (s, 6H) | LCMS-X1: 509.2 [M + H]⁺; Rₜ = 6.03 min |
| 28 | 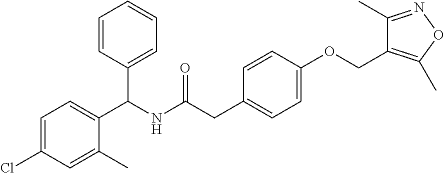 [SMILES: Cc1cc(Cl)ccc1C(NC(=O)Cc1ccc(OCc2c(C)noc2C)cc1)c1ccccc1] N-((4-chloro-2- methylphenyl)(phenyl)methyl)-2- (4-((3,5-dimethylisoxazol-4- yl)methoxy)phenyl)acetamide | ¹H NMR (400 MHz, DMSO-d₆) δ ppm 8.91-8.93 (d, 1 H), 7.32-7.34 (d, 2 H), 7.23-7.29 (m, 3 H), 7.17- 7.20 (m, 4 H), 7.10-7.12 (d, 1 H), 6.92-6.94 (d, 2 H), 6.17-6.19 (d, 1 H), 5.88 (s, 2 H), 3.45 (s, 2 H), 2.39 (s, 3 H), 2.20 (s, 6H) | LCMS-X1: 475.2 [M + H]⁺; Rₜ = 7.10 min |
| 29 | 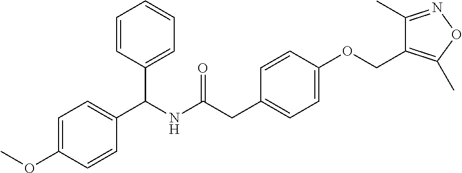 [SMILES: COc1ccc(C(NC(=O)Cc2ccc(OCc3c(C)noc3C)cc2)c2ccccc2)cc1] 2-(4-((3,5-dimethylisoxazol-4- yl)methoxy)phenyl)-N-((4- methoxyphenyl)(phenyl)methyl) acetamide | ¹H NMR (400 MHz, DMSO-d₆) δ ppm 8.89-8.92 (d, 1 H), 7.29-7.33 (m, 2 H), 7.15-7.24 (m, 7 H), 6.87- 6.93 (m, 4 H), 6.02-6.04 (d, 1 H), 4.87 (s, 2 H), 3.72 (s, 3 H), 3.41- 3.43 (s, 2 H), 2.38 (s, 3 H), 2.19 (s, 3H), | LCMS-X1: 457.5 [M + H]⁺; Rₜ = 6.47 min |
| 30 | 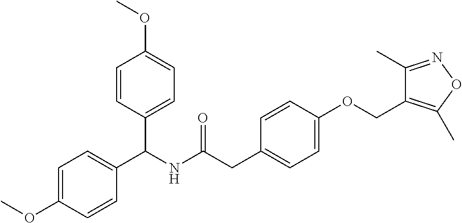 [SMILES: COc1ccc(C(NC(=O)Cc2ccc(OCc3c(C)noc3C)cc2)c2ccc(OC)cc2)cc1] N-(bis(4-methoxyphenyl)methyl)- 2-(4-((3,5-dimethylisoxazol-4- yl)methoxy)phenyl)acetamide | ¹H NMR (400 MHz, DMSO-d₆) δ ppm 8.83-8.85 (d, 1 H), 7.18-7.20 (d, 2 H), 7.13-7.15 (d, 4 H), 6.91- 6.93 (d, 2 H), 6.85-6.88 (d, 4 H), 5.96-5.98 (d, 1 H), 4.87 (s, 2 H), 3.72 (s, 6 H), 3.41-3.44 (s, 2 H), 2.38 (s, 3 H), 2.19 (s, 3 H). | LCMS-X1: 487.3 [M + H]⁺; Rₜ = 6.41 min |
| 31 | 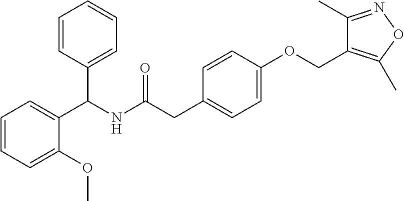 [SMILES: COc1ccccc1C(NC(=O)Cc1ccc(OCc2c(C)noc2C)cc1)c1ccccc1] 2-(4-((3,5-dimethylisoxazol-4- yl)methoxy)phenyl)-N-((2- methoxyphenyl)(phenyl)methyl) acetamide | ¹H NMR (400 MHz, DMSO-d₆) δ ppm 8.75-8.77 (d, 1 H), 7.25-7.30 (m, 4 H), 7.15-7.23 (m, 5 H), 6.91- 6.98 (m, 4 H), 6.36-6.38 (d, 1 H), 4.87 (s, 2 H), 3.70 (s, 3 H), 3.35 (s, 2 H), 2.38 (s, 3 H), 2.20 (s, 3 H). | LCMS-X1: 457.3 [M + H]⁺; Rₜ = 6.54 min |
| 32 | 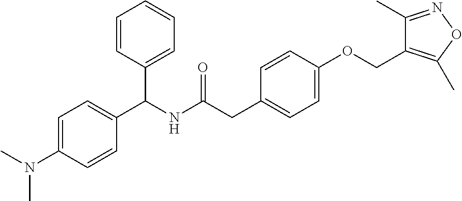 [SMILES: Cc1noc(C)c1COc1ccc(CC(=O)NC(c2ccccc2)c2ccc(N(C)C)cc2)cc1] N-((4-(dimethylamino)phenyl) (phenyl)methyl)-2-(4-((3,5- dimethylisoxazol-4-yl) methoxy)phenyl)acetamide | ¹H NMR (400 MHz, DMSO-d₆) δ ppm 8.82-8.84 (d, 1 H), 7.27-7.29 (d, 2 H), 7.18-7.23 (t, 4 H), 7.03-7.05 (d, 2 H), 6.93-6.91 (d, 2 H) 6.65-6.67 (d, 2 H), 5.94-5.96 (d, 1 H), 4.87 (s, 2 H), 3.36-3.45 (d, 2 H), 2.84 (s, 6 H), 2.38 (s, 3 H), 2.19 (s, 3 H). | LCMS-X1: 470.3 [M + H]⁺; Rₜ = 4.20 min |
| 33 | 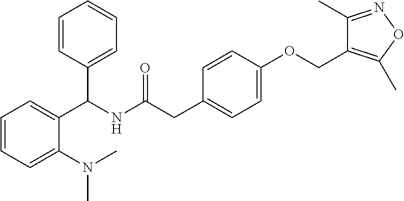 [SMILES: Cc1noc(C)c1COc1ccc(CC(=O)NC(c2ccccc2)c2ccccc2N(C)C)cc1] N-((2-(dimethylamino)phenyl) (phenyl)methyl)-2-(4-((3,5- dimethylisoxazol-4- yl)methoxy)phenyl)acetamide | ¹H NMR (400 MHz, DMSO-d₆) δ ppm 8.83-8.85 (d, 1 H), 7.25-7.28 (m, 4 H), 7.18-7.23 (m, 4 H), 7.05- 7.13 (m, 3 H), 6.91-6.93 (d, 2 H), 6.62-6.64 (d, 1 H), 4.87 (s, 2 H), 3.58 (s, 2 H), 2.51 (s, 6 H), 2.38 (s, 3H), 2.19 (s, 3 H). | LCMS-X1: 470.3 [M + H]⁺; Rₜ = 6.23 min |
| 34 | 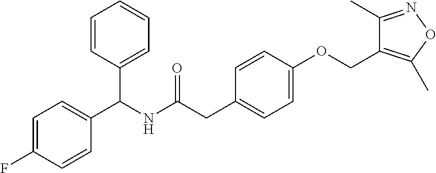 [SMILES: Cc1noc(C)c1COc1ccc(CC(=O)NC(c2ccccc2)c2ccc(F)cc2)cc1] 2-(4-((3,5-dimethylisoxazol-4- yl)methoxy)phenyl)-N-((4- fluorophenyl)(phenyl)methyl) acetamide | ¹H NMR (400 MHz, DMSO-d₆) δ ppm 8.98-9.00 (d, 1H), 7.28-7.34 (m, 4 H), 7.24-7.26 (d, 3 H), 7.13- 7.23 (m, 4 H), 6.91-6.93 (d, 2 H), 6.08-6.10 (d, 1 H), 4.87 (s, 2 H), 3.42-3.47 (s, 2 H), 2.38 (s, 3H), 2.19 (s, 3 H). | LCMS-X1: 445.3 [M + H]⁺; Rₜ = 6.55 min |
| 35 | 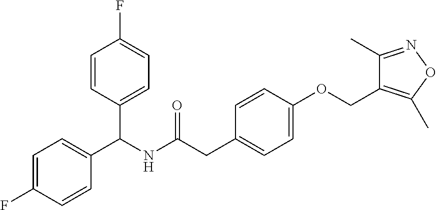 [SMILES: Cc1noc(C)c1COc1ccc(CC(=O)NC(c2ccc(F)cc2)c2ccc(F)cc2)cc1] N-(bis(4-fluorophenyl)methyl)-2- (4-((3,5-dimethylisoxazol-4- yl)methoxy)phenyl)acetamide | ¹H NMR (400 MHz, DMSO-d₆) δ ppm 8.98-9.00 (d, 1 H), 7.27-7.31 (m, 4 H), 7.14-7.20 (m, 6 H), 6.91- 6.93 (d, 2 H), 6.10-6.12 (d, 1 H), 4.87 (s, 2 H), 3.46 (s, 2 H), 2.38 (s, 3 H), 2.29 (s, 3 H). | LCMS-T1: 463.1 [M + H]⁺; Rₜ = 6.37 min |
| 36 | 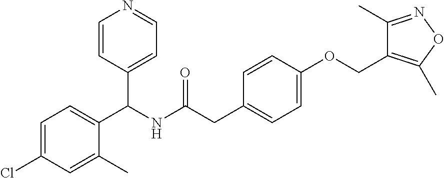 [SMILES: Cc1cc(Cl)ccc1C(NC(=O)Cc1ccc(OCc2c(C)noc2C)cc1)c1ccncc1] N-((4-chloro-2- methylphenyl)(pyridin-4- yl)methyl)-2-(4-((3,5- dimethylisoxazol-4- yl)methoxy)phenyl)acetamide | ¹H NMR (400 MHz, DMSO-d₆) δ ppm 8.96-8.98 (d, 1 H), 8.51-8.53 (dd, 2 H), 7.32-7.33 (d, 1 H), 7.18- 7.26 (m, 5 H), 6.92-6.98 (m, 3 H), 6.19-6.21 (d, 1 H), 4.88 (s, 2 H), 3.47 (s, 2 H), 2,39 (s, 3 H), 2.24 (s, 6 H). | LCMS-X1: 476.6 [M + H]⁺; Rₜ = 5.10 min |
| 37 | 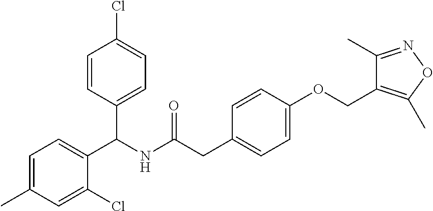 [SMILES: Cc1ccc(C(NC(=O)Cc2ccc(OCc3c(C)noc3C)cc2)c2ccc(Cl)cc2)c(Cl)c1] N-((2-chloro-4-methylphenyl)(4- chlorophenyl)methyl)-2-(4-((3,5- dimethylisoxazol-4- yl)methoxy)phenyl)acetamide | ¹H NMR (400 MHz, DMSO-d₆) δ ppm 8.97-8.99 (d, 1H), 7.38-7.40 (d, 2 H), 7.25-7.28 (m, 2 H), 7.15- 7.19 (m, 5 H), 6.91-6.94 (d, 2 H), 6.32-6.34 (d, 1 H), 4.87 (s, 2 H), 3.45 (s, 2 H), 2.38 (s, 3H), 2.28 (s, 3 H), 2.20 (s, 3 H). | LCMS-X1: 509.2 [M + H]⁺; Rₜ = 5.47 min |
| 38 | 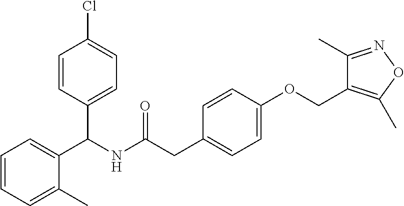 [SMILES: Cc1ccccc1C(NC(=O)Cc1ccc(OCc2c(C)noc2C)cc1)c1ccc(Cl)cc1] N-((4-chlorophenyl)(o- tolyl)methyl)-2-(4-((3,5- dimethylisoxazol-4-yl)methoxy) phenyl)acetamide | ¹H NMR (400 MHz, DMSO-d₆) δ ppm 8.90-8.92 (d, 1H), 7.36-7.40 (t, 2 H), 7.11-7.33 (m, 7 H), 7.04- 7.06 (d, 1 H), 6.91-6.93 (d, 2 H), 6.20-6.22 (d, 1 H), 4.78 (s, 2 H), 3.45 (s, 2 H), 2.38 (s, 3 H), 2.22 (s, 6 H). | LCMS-X1: 475.5 [M + H]⁺; Rₜ = 7.04 min |
| 39 | 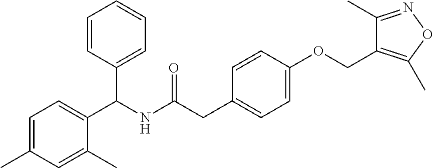 [SMILES: Cc1ccc(C(NC(=O)Cc2ccc(OCc3c(C)noc3C)cc2)c2ccccc2)c(C)c1] 2-(4-((3,5-dimethylisoxazol-4- yl)methoxy)phenyl)-N-((2,4- dimethylphenyl)(phenyl)methyl) acetamide | ¹H NMR (400 MHz, DMSO-d₆) δ ppm 8.82-8.84 (d, 1 H), 7.29-7.33 (m, 2 H), 7.22-7.26 (m, 1 H), 7.15- 7.20 (m, 4 H), 6.91-6.99 (m, 5 H), 6.16-6.18 (d, 1 H), 4.87 (s, 2 H), 3.41-3.46 (s, 2 H), 2.38 (s, 3 H), 2.37 (s, 3H), 2.20 (s, 3 H), 2.15 (s, 3 H). | LCMS-X1: 455.3 [M + H]⁺; Rₜ = 7.02 min |
| 40 | 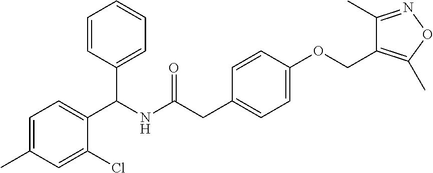 [SMILES: Cc1ccc(C(NC(=O)Cc2ccc(OCc3c(C)noc3C)cc2)c2ccccc2)c(Cl)c1] N-((2-chloro-4- methylphenyl)(phenyl)methyl)-2- (4-((3,5-dimethylisoxazol-4- yl)methoxy)phenyl)acetamide | ¹H NMR (400 MHz, DMSO-d₆) δ ppm 8.96-8.98 (s, 1 H), 7.24-7.34 (m, 5 H), 7.13-7.20 (m, 5 H), 6.91- 6.93 (d, 2 H), 6.33-6.35 (d, 2 H), 4.87 (s, 2 H), 3.42-3.49 (m, 2 H), 2.38 (s, 3 H), 2.33 (s, 3 H), 2.19 (s, 3 H). | LCMS-X1: 475.3 [M + H]⁺; Rₜ = 6.95 min |

Using essentially the same procedure as described in example 4, the following compounds in table 2 were made.

TABLE 2

| Ex. | Structure/Name | NMR | LCMS |
|---|---|---|---|
| 41 | 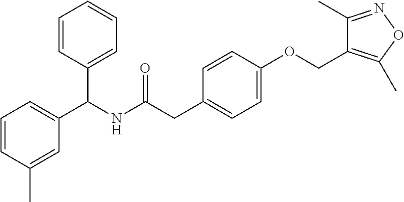 [SMILES: Cc1cccc(C(NC(=O)Cc2ccc(OCc3c(C)noc3C)cc2)c2ccccc2)c1] 2-(4-((3,5-dimethylisoxazol-4- yl)methoxy)phenyl)-N- (phenyl(m-tolyl)methyl)acetamide | ¹H NMR (500 MHz, MeOH-d₄) δ ppm 7.21-7.06 (m, 8H), 6.97-6.82 (m, 5H), 6.01 (s, 1H), 5.39 (s, 1H), 4.78 (s, 2H), 3.21 (s, 2H), 2.29 (s, 3H), 2.17 (s, 3H), 2.16 (s, 3H). | LCMS-P1: 441 [M + H]⁺; Rₜ: 1.699 min. |
| 42 | 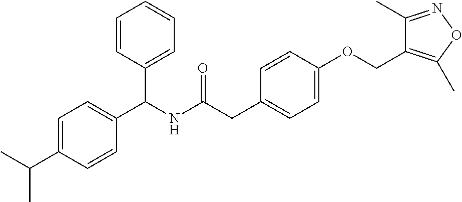 [SMILES: Cc1noc(C)c1COc1ccc(CC(=O)NC(c2ccccc2)c2ccc(C(C)C)cc2)cc1] 2-(4-((3,5-dimethylisoxazol-4- yl)methoxy)phenyl)-N-((4- isopropylphenyl)(phenyl)methyl) acetamide | ¹H NMR (400 MHz, MeOH-d₄) δ ppm 8.74 (d, J = 8.4 Hz, 1H), 7.21- 7.06 (m, 9H), 7.00 (d, J = 8.4 Hz, 2H), 6.82 (d, J = 8.8 Hz, 2H), 6.01 (s, 1H), 4.77 (s, 2H), 3.43 (s, 2H), 2.83-2.72 (m, 1H), 2.28 (s, 3H), 2.15 (s, 3H), 1.13 (s, 3H), 1.12 (s, 3H). | LCMS-P1: 491 [M + Na]⁺; Rₜ: 1.796 min. |
| 43 | 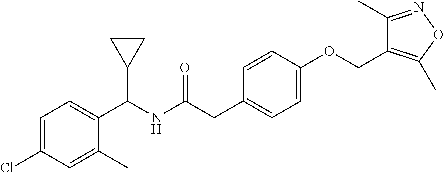 [SMILES: Cc1cc(Cl)ccc1C(NC(=O)Cc1ccc(OCc2c(C)noc2C)cc1)C1CC1] N-((4-chloro-2- methylphenyl)(cyclopropyl) methyl)- 2-(4-((3,5-dimethylisoxazol-4- yl)methoxy)phenyl)acetamide | ¹H NMR (400 MHz, DMSO-d₆) δ ppm 8.45-8.47 (d, 1 H), 7.46-7.48 (d, 1 H), 7.23-7.26 (d, 1 H), 7.20 (s, 1 H), 7.13-7.15 (d, 2 H), 6.89- 6.92 (d, 2 H), 4.86 (s, 2 H), 4.54- 4.58 (t, 1 H), 2.33 (s, 3 H), 2.19 (s, 8 H), 1.16-1.23 (m, 1 H), 0.49-0.51 (t, 1 H), 0.47 (s, 1 H), 0.43 (s, 1 H), 0.40-0.41 (d, 1 H). | LCMS-X1: 439.3 [M + H]⁺; Rₜ = 6.80 min |
| 44 | 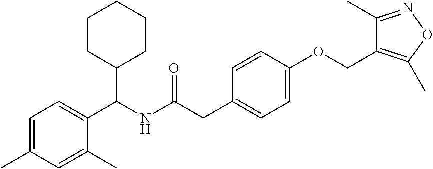 [SMILES: Cc1ccc(C(NC(=O)Cc2ccc(OCc3c(C)noc3C)cc2)C2CCCCC2)c(C)c1] N-(cyclohexyl(2,4- dimethylphenyl)methyl)-2-(4- ((3,5-dimethylisoxazol-4- yl)methoxy)phenyl)acetamide | ¹H NMR (400 MHz, CDCl₃) δ ppm 8.87 (d, 1H), 8.19 (t, 1H), 7.68-7.60 (m, 2H), 7.30-7.22 (m, 2H), 7.16 (d, 2H), 6.95-6.91 (m, 3H), 6.79 (d, 1H), 5.61 (d, 1H), 4.96 (t, 1H), 4.80 (s, 2H), 3.50 (q, 2H), 2.41 (s, 3H), 2.34 (s, 3H), 2.30 (s, 3H), 2.27 (s, 3H), 1.72 (m, 2H), 1.62 (m, 2H), 1.35 (m, 2H), 1.08 (m, 2H), 0.92 (m, 2H). | LCMS-T1: 461.3 [M + H]⁺; Rₜ = 7.19 min |
| 45 | 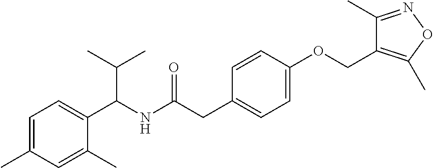 [SMILES: Cc1ccc(C(NC(=O)Cc2ccc(OCc3c(C)noc3C)cc2)C(C)C)c(C)c1] 2-(4-((3,5-dimethylisoxazol-4- yl)methoxy)phenyl)-N-(1-(2,4- dimethylphenyl)-2- methylpropyl)acetamide | ¹H NMR (400 MHz, CDCl₃) δ ppm 7.18 (m, 2H), 6.95-6.92 (m, 4H), 6.79 (d, 2H), 5.61 (d, 1H), 4.94 (t, 1H), 4.80 (s, 2H), 3.50 (q, 2H), 2.41 (s, 3H), 2.36 (s, 3H), 2.30 (s, 3H), 2.27 (s, 3H), 0.86 (d, 3H), 0.76 (d, 3H). | LCMS-T1: 421.3 [M + H]⁺; Rₜ = 6.54 min |
| 46 | 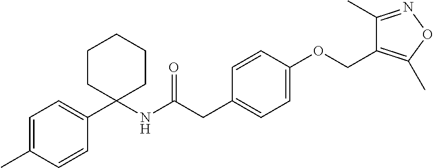 [SMILES: Cc1ccc(C2(NC(=O)Cc3ccc(OCc4c(C)noc4C)cc3)CCCCC2)cc1] 2-(4-((3,5-dimethylisoxazol-4- yl)methoxy)phenyl)-N-(1-(p- tolyl)cyclohexyl)acetamide | ¹H NMR (400 MHz, CDCl₃) δ ppm 7.41 (d, 2H) 7.24-7.09 (m, 5H), 6.96 (d, 1H), 5.46 (1H), 4.81 (s, 2H), 3.51 (s, 2H), 2.41 (3H, s), 2.30 (6H, s), 2.27 (2H, t), 1.75- 1.58 (m, 6H), 1.25 (2H, t) | LCMS-T1: 433.2 [M + H]⁺; Rₜ = 6.54 min |
| 47 | 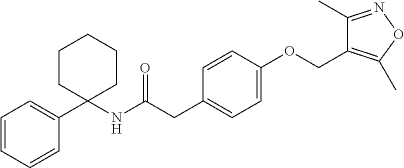 [SMILES: Cc1noc(C)c1COc1ccc(CC(=O)NC2(c3ccccc3)CCCCC2)cc1] 2-(4-((3,5-dimethylisoxazol-4- yl)methoxy)phenyl)-N-(1- phenylcyclohexyl)acetamide | ¹H NMR (CDCl₃) δ ppm 7.30-7.28 (3H, m), 7.26-7.24 (3H, m), 7.20 (1H, m), 6.95 (2H, dt), 5.48 (1H, s), 4.80 (2H, s), 3.52 (2H, s), 2.40 (2H, s), 2.39 (1H, t), 2.29 (2H, s), 2.26 (2H, t), 1.69 (2H, t), 1.60 (2H, t), 1.56 (2H, s), 1.24 (3H, t). | MS 419.2 [M + H]⁺. |
| 48 | 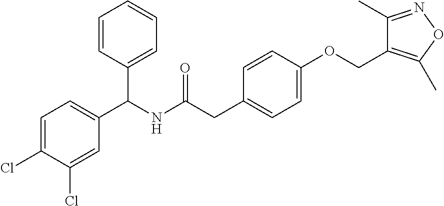 [SMILES: Cc1noc(C)c1COc1ccc(CC(=O)NC(c2ccccc2)c2ccc(Cl)c(Cl)c2)cc1] N-((3,4- dichlorophenyl)(phenyl)methyl)-2- (4-((3,5-dimethylisoxazol-4- yl)methoxy)phenyl)acetamide | ¹H NMR (CDCl₃) δ ppm 7.34 (1H, d), 7.30 (2H, d), 7.29 (1H, t), 7.25 (1H, s), 7.22 (1H, s), 7.19 (1H, s), 7.16 (1H, d), 7.06 (2H, dd), 6.95- 6.92 (2H, m), 6.16 (1H, d), 5.93 (1H, d), 4.77 (2H, s), 3.58 (2H, s), 2.39 (3H, s), 2.28 (3H, s). | MS 496.1 [M + H]⁺. |
| 49 | 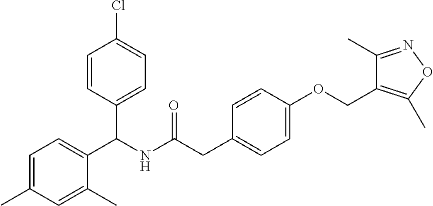 [SMILES: Cc1ccc(C(NC(=O)Cc2ccc(OCc3c(C)noc3C)cc2)c2ccc(Cl)cc2)c(C)c1] N-((4-chlorophenyl)(2,4- dimethylphenyl)methyl)-2-(4- ((3,5-dimethylisoxazol-4- yl)methoxy)phenyl)acetamide | ¹H NMR (DMSO-d₆) δ ppm 8.81 (1H, d), 7.35 (2H, d), 7.17-7.14 (4H, m), 6.96-6.88 (5H, m), 6.14 (1H, d), 4.85 (2H, s), 3.41 (2H, s), 2.36 (3H, s), 2.21 (3H, s), 2.17 (3H, s), 2.12 (3H, s). | MS 490.2 [M + H]⁺. |
| 50 | 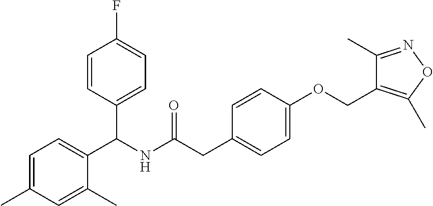 [SMILES: Cc1ccc(C(NC(=O)Cc2ccc(OCc3c(C)noc3C)cc2)c2ccc(F)cc2)c(C)c1] 2-(4-((3,5-dimethylisoxazol-4- yl)methoxy)phenyl)-N-((2,4- dimethylphenyl)(4- fluorophenyl)methyl)acetamide | ¹H NMR (DMSO-d₆) δ ppm 8.82 (1H, d), 7.19-7.09 (6H, m), 6.95 (2H, d), 6.94 (1H, s), 6.90 (2H, d), 6.15 (1H, d), 4.85 (2H, s), 3.42 (2H, s), 2.36 (3H, s), 2.21 (3H, s), 2.17 (3H, s), 2.12 (3H, s). | MS 473.2 [M + H]⁺. |
| 51 | 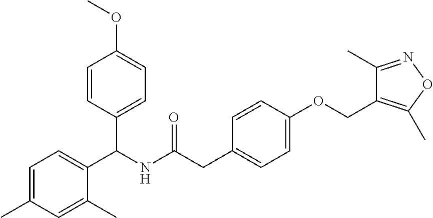 [SMILES: COc1ccc(C(NC(=O)Cc2ccc(OCc3c(C)noc3C)cc2)c2ccc(C)cc2C)cc1] 2-(4-((3,5-dimethylisoxazol-4- yl)methoxy)phenyl)-N-((2,4- dimethylphenyl)(4- methoxyphenyl)methyl)acetamide | ¹H NMR (400 MHz, CDCl₃) δ 7.20 (2H, d), 6.97 (2H, d), 6.96 (1H, s), 6.93-6.88 (3H, m), 6.82-6.77 (3H, m), 6.29 (1H, d), 5.92 (1H, d), 4.77 (2H, s), 3.76 (3H, s), 3.55 (2H, s), 2.38 (3H, s), 2.28 (3H, s), 2.27 (3H, s), 2.18 (3H, s). | LCMS 507.2 (MNa⁺). |
| 52 | 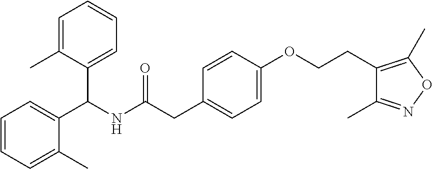 [SMILES: Cc1ccccc1C(NC(=O)Cc1ccc(OCCc2c(C)noc2C)cc1)c1ccccc1C] N-(di-o-tolylmethyl)-2-(4-(2- (3,5-dimethylisoxazol-4- yl)ethoxy)phenyl)acetamide | ¹H NMR (400 MHz, CDCl₃) δ ppm 7.16-7.12 (6H, m), 7.08-7.04 (2H, m), 6.84 (2H, d), 6.79 (2H, d), 6.42 (1H, d), 5.92 (1H, d), 3.97 (2H, t), 3.51 (2H, s), 2.76 (2H, t), 2.35 (3H, s) 2.25 (3H, s), 2.19 (6H, s). | MS 469.3 [M + H]⁺. |
| 53 | 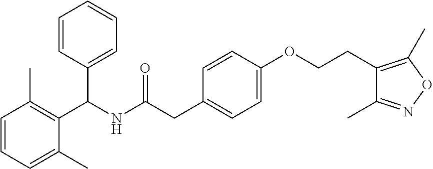 [SMILES: Cc1cccc(C)c1C(NC(=O)Cc1ccc(OCCc2c(C)noc2C)cc1)c1ccccc1] 2-(4-(2-(3,5-dimethylisoxazol-4- yl)ethoxy)phenyl)-N-((2,6- dimethylphenyl)(phenyl)methyl) acetamide | ¹H NMR (400 MHz, CDCl₃) δ ppm 7.68 (2H, d), 7.49 (1H, t), 7.39 (2H, td), 7.24 (2H, m), 7.08 (2H, d), 6.86 (2H, d), 6.70 (2H, d), 3.93 (2H, t), 3.51 (2H, s), 2.74 (2H, t), 2.34 (3H, s), 2.25 (3H, s), 2.18 (6H, s). | MS 468.2 [M + H]⁺. |
| 54 | 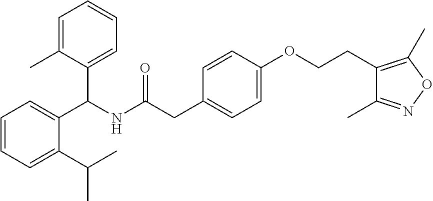 [SMILES: Cc1ccccc1C(NC(=O)Cc1ccc(OCCc2c(C)noc2C)cc1)c1ccccc1C(C)C] 2-(4-(2-(3,5-dimethylisoxazol-4- yl)ethoxy)phenyl)-N-((2- isopropylphenyl)(o-tolyl)methyl) acetamide | ¹H NMR (400 MHz, CDCl₃) δ ppm 7.29 (1H, d), 7.24 (1H, d), 7.16-7.12 (4H, m), 7.08 (1H, t), 7.02 (1H, t), 6.89 (1H, d), 6.80- 6.76 (3H, m), 6.56 (1H, d), 5.81 (1H, d), 3.96 (2H, t), 3.53 (2H, s), 3.05 (1H, m), 2.76 (2H, t), 2.34 (3H, s), 2.25 (3H, s), 2.16 (3H, s), 1.20 (3H, d), 1.06 (3H, d). | MS 497.3 [M + H]⁺. |
| 55 | 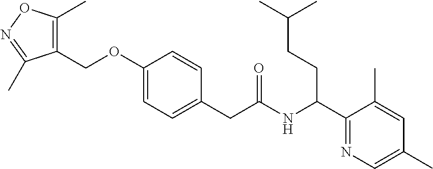 [SMILES: Cc1cnc(C(CCC(C)C)NC(=O)Cc2ccc(OCc3c(C)noc3C)cc2)c(C)c1] 2-(4-((3,5-dimethylisoxazol-4- yl)methoxy)phenyl)-N-(1-(3,5- dimethylpyridin-2-yl)-4- methylpentyl)acetamide | ¹H NMR (CDCl₃, 400 MHz): δ 9.38 (s, 1H), 8.36 (s, 1H), 7.81 (s, 1H), 7.24 (d, J = 8.0 Hz, 2H), 6.85 (d, J = 8.0 Hz, 2H), 5.19 (s, 1H), 4.75 (s, 2H), 3.51 (s, 2H), 2.61 (s, 3H), 2.45 (s, 3H), 2.38 (s, 3H), 2.26 (s, 3H), 2.06-1.93 (m, 1H), 1.83-1.72 (m, 1H), 1.54-1.51 (m, 1H), 1.33-1.25 (m, 3H), 1.13-1.04 (m, 3H), 0.85 (s, 3H), 0.82 (s, 3H). | LCMS-A024: 450.7 [M + H]⁺. Rt: 1.44 min. |

Using essentially the same procedure as described in example 7, the following compounds in table 3 were made.

TABLE 3

| Ex. | Structure/Name | NMR | LCMS |
|---|---|---|---|
| 56 | 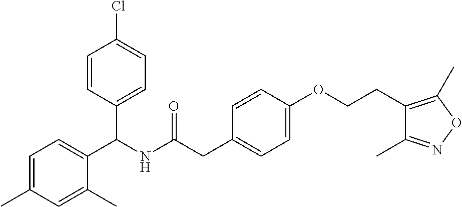 [SMILES: Cc1ccc(C(NC(=O)Cc2ccc(OCCc3c(C)noc3C)cc2)c2ccc(Cl)cc2)c(C)c1] N-((4-chlorophenyl)(2,4- dimethylphenyl)methyl)-2-(4-(2- (3,5-dimethylisoxazol-4- yl)ethoxy)phenyl)acetamide | ¹H NMR (400 MHz, DMSO-d₆) δ ppm 8.80-8.82 (d, 1 H), 7.36-7.38 (d, 2 H), 7.14-7.18 (t, 4 H), 6.90- 6.98 (m, 2 H), 6.81-6.83 (d, 2 H), 6.14-6.17 (d, 1 H), 3.97-4.00 (t, 2 H), 3.39-3.42 (d, 2 H), 2.72-2.75 (t, 2H), 2.32 (s, 3 H), 2.23 (s, 3 H), 2.20 (s, 3 H), 2.14 (s, 3 H). | LCMS-X1: 503.5 [M + H]⁺. Rₜ = 5.52 min |
| 57 | 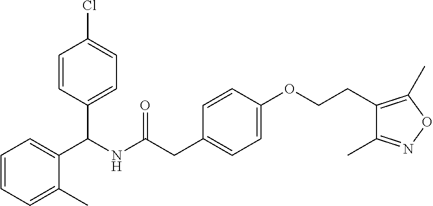 [SMILES: Cc1ccccc1C(NC(=O)Cc1ccc(OCCc2c(C)noc2C)cc1)c1ccc(Cl)cc1] N-((4-chlorophenyl)(o- tolyl)methyl)-2-(4-(2-(3,5- dimethylisoxazol-4-yl)ethoxy) phenyl)acetamide | ¹H NMR (400 MHz, DMSO-d₆) δ ppm 8.86-8.88 (d, 1H), 7.38-7.40 (d, 2 H), 7.14-7.20 (m, 7 H), 7.03- 7.05 (d, 1 H), 6.81-6.83 (d, 2 H), 6.19-6.21 (d, 1 H), 3.97-4.00 (t, 2 H), 3.43 (s, 2 H), 2.72-2.75 (t, 2 H), 2.32 (s, 3 H), 2.19-2.20 (s, 6 H). | LCMS-X1: 488.5 [M + H]⁺. Rₜ = 7.04 min |
| 58 | 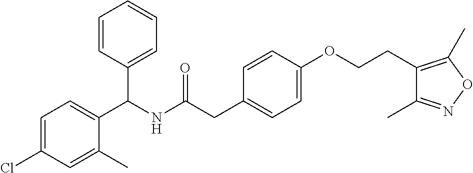 [SMILES: Cc1cc(Cl)ccc1C(NC(=O)Cc1ccc(OCCc2c(C)noc2C)cc1)c1ccccc1] N-((4-chloro-2- methylphenyl)(phenyl)methyl)-2- (4-(2-(3,5-dimethylisoxazol-4- yl)ethoxy)phenyl)acetamide | ¹H NMR (400 MHz, DMSO-d₆) δ ppm 8.90-8.88 (d, 1 H), 7.31-7.35 (t, 2 H), 7.22-7.28 (m, 3 H), 7.15- 7.17 (m, 3 H), 7.09-7.11 (d, 1 H), 6.81-6.84 (t, 2 H), 6.16-6.18 (d, 1 H), 3.98-4.01 (t, 2 H), 3.43 (s, 2 H), 2.72-2.76 (t, 2H), 2.33 (s, 3 H), 2.20 (s, 6 H). | LCMS-T1: 490.1 [M + H]⁺; Rₜ = 6.96 min |
| 59 | 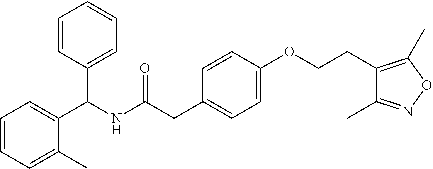 [SMILES: Cc1ccccc1C(NC(=O)Cc1ccc(OCCc2c(C)noc2C)cc1)c1ccccc1] 2-(4-(2-(3,5-dimethylisoxazol-4- yl)ethoxy)phenyl)-N- (phenyl(o-tolyl)methyl)acetamide | ¹H NMR (400 MHz, DMSO-d₆) δ ppm 8.85-8.88 (d, 1 H), 7.30-7.33 (m, 2 H), 7.23-7.26 (m, 1 H), 7.15-7.18 (m, 7 H), 7.09-7.10 (d, 1 H), 6.81-6.83 (d, 2 H), 6.20-6.22 (d, 1H), 3.97-4.00 (t, 2 H), 3.39-3.43 (s, 2 H), 2.72-2.75 (t, 2H), 2.32 (s, 3 H), 2.18-2.20 (d, 6 H). | LCMS-X1: 455.5 [M + H]⁺; Rₜ = 5.05 min |
| 60 | 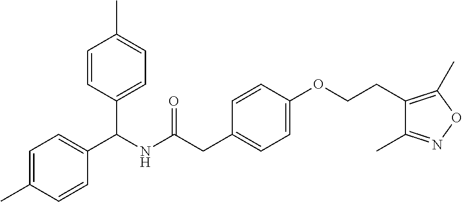 [SMILES: Cc1ccc(C(NC(=O)Cc2ccc(OCCc3c(C)noc3C)cc2)c2ccc(C)cc2)cc1] N-(di-p-tolylmethyl)-2-(4-(2-(3,5- dimethylisoxazol-4- yl)ethoxy)phenyl)acetamide | ¹H NMR (400 MHz, DMSO-d₆) δ ppm 8.82-8.84 (d, 1H), 7.10-7.25 (m, 10 H), 6.81-6.83 (d, 2 H), 5.97-5.99 (d, 1 H), 3.97-4.00 (t, 2 H), 3.40-3.43 (s, 2 H), 2.72-2.75 (t, 2 H), 2.32 (s, 3 H), 2.25 (s, 6H), 2.19 (s, 3 H). | LCMS-X1: 469.5 [M + H]⁺; Rₜ = 7.10 min |
| 61 | 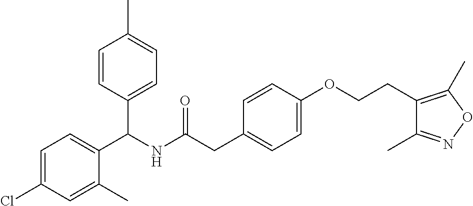 [SMILES: Cc1ccc(C(NC(=O)Cc2ccc(OCCc3c(C)noc3C)cc2)c2ccc(Cl)cc2C)cc1] N-((4-chloro-2-methylphenyl)(p- tolyl)methyl)-2-(4-(2-(3,5- dimethylisoxazo-4- yl)ethoxy)phenyl)acetamide | ¹H NMR (400 MHz, DMSO-d₆) δ ppm 8.82-8.84 (d, 1 H), 7.19-7.28 (m, 3 H), 7.08-7.16 (m, 4 H), 7.03-7.05 (d, 2 H), 6.81-6.83 (d, 2 H), 6.10-6.13 (d, 1 H), 3.97-4.00 (t, 2 H), 3.38-3.41 (s, 2 H), 2.72-2.75 (t, 2H), 2.23 (s, 3 H), 2.20 (s, 3 H), 2.14 (s, 3H), 2.08 (s, 3H). | LCMS-X1: 503.4 [M + H]⁺; Rₜ = 5.56 min |
| 62 | 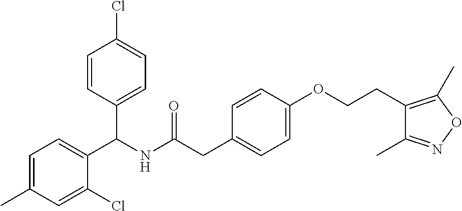 [SMILES: Cc1ccc(C(NC(=O)Cc2ccc(OCCc3c(C)noc3C)cc2)c2ccc(Cl)cc2)c(Cl)c1] N-((2-chloro-4-methylphenyl)(4- chlorophenyl)methyl)-2-(4-(2-(3,5- dimethylisoxazol-4- yl)ethoxy)phenyl)acetamide | ¹H NMR (400 MHz, DMSO-d₆) δ ppm 8.95-8.97 (d, 1 H), 7.38-7.40 (d, 2 H), 7.24-7.27 (t, 2 H), 7.13-7.16 (m, 4 H), 6.81-6.83 (d, 2 H), 6.30-6.32 (d, 1 H), 3.97-4.00 (t, 2 H), 3.41-3.46 (t, 2 H), 2.72-2.75 (m, 2H), 2.28-2.32 (d, 6 H), 2.19 (s, 3 H). | LCMS-X1: 523.4 [M + H]⁺; Rₜ = 5.51 min |
| 63 | 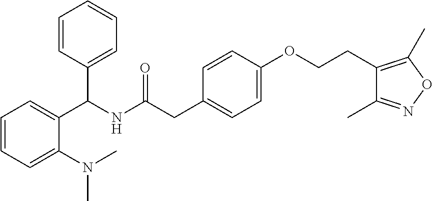 [SMILES: Cc1noc(C)c1CCOc1ccc(CC(=O)NC(c2ccccc2)c2ccccc2N(C)C)cc1] N-((2-(dimethylamino)phenyl) (phenyl)methyl)-2-(4-(2-(3,5- dimethylisoxazol-4- yl)ethoxy)phenyl)acetamide | ¹H NMR (400 MHz, DMSO-d₆) δ ppm 8.80-8.82 (d, 1H), 7.16-7.28 (m, 8 H), 7.04-7.12 (m, 3 H), 6.81-6.83 (d, 2 H), 6.61-6.63 (d, 1 H), 3.97-4.00 (t, 2 H), 3.39 (s, 2 H), 2.72-2.75 (t, 2 H), 2.53 (s, 6H), 2.32 (s, 3 H), 2.19 (s, 3 H). | LCMS-X1: 484.6 [M + H]⁺; Rₜ = 6.43 min |
| 64 | 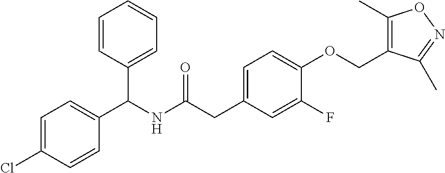 [SMILES: Cc1noc(C)c1COc1ccc(CC(=O)NC(c2ccccc2)c2ccc(Cl)cc2)cc1F] N-((4- chlorophenyl)(phenyl)methyl)-2- (4-((3,5-dimethylisoxazol-4- yl)methoxy)-3- fluorophenyl)acetamide | ¹H NMR (400 MHz, DMSO-d₆) δ ppm 9.00-9.02 (d, 1H), 7.17-7.40 (m, 10 H), 7.09-7.12 (dd, 1 H), 7.02-7.04 (d, 1 H), 6.08-6.10 (d, 1 H), 4.96 (s, 2 H), 3.49 (s, 2 H), 2.36 (s, 3 H), 2.20 (s, 3H). | LCMS-X1: 479.2 [M + H]⁺; Rₜ = 6.88 min |
| 65 | 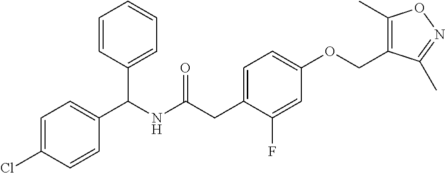 [SMILES: Cc1noc(C)c1COc1ccc(CC(=O)NC(c2ccccc2)c2ccc(Cl)cc2)c(F)c1] N-((4- chlorophenyl)(phenyl)methyl)-2- (4-((3,5-dimethylisoxazol-4- yl)methoxy)-2- fluorophenyl)acetamide | ¹H NMR (400 MHz, DMSO-d₆) δ ppm 9.02-9.04 (d, 1H), 7.20-7.41 (m, 9 H), 6.85-6.89 (dd, 1 H), 6.77-6.80 (dd, 1 H), 6.10-6.12 (d, 1 H), 4.90 (s, 2 H), 3.54 (s. 2 H), 2.39 (s, 3 H), 2.20 (s, 3H). | LCMS-X1: 479.4 [M + H]⁺; Rₜ = 6.98 min |

Using essentially the same procedure as described in example 9, the following compounds in table 4 were made.

TABLE 4

| Ex. | Structure/Name | NMR | LCMS |
|---|---|---|---|
| 66 | 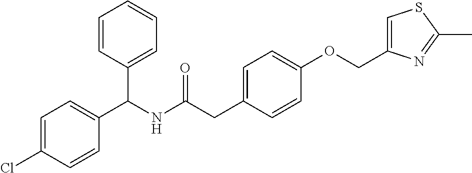 [SMILES: Cc1nc(COc2ccc(CC(=O)NC(c3ccccc3)c3ccc(Cl)cc3)cc2)cs1] N-((4- chlorophenyl)(phenyl)methyl)-2-(4- ((2-methylthiazol-4- yl)methoxy)phenyl)acetamide | ¹H NMR (400 MHz, DMSO-d₆) δ ppm 8.99-9.02 (d, 1 H), 7.52 (s, 1 H), 7.38-7.40 (d, 2 h), 7.31-7.19 (m, 7 H), 6.93-6.95 (d, 2 H), 6.07-6.09 (d, 1 H), 5.06 (s, 2 H), 3.47 (s, 2 H), 2.65 (s, 3 H). | LCMS-X1: 463.1 [M + H]⁺; Rₜ = 3.41 min |
| 67 | 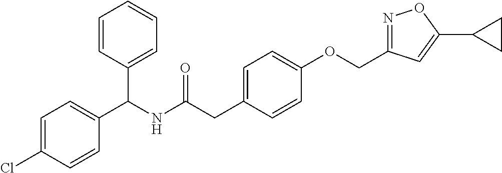 [SMILES: O=C(Cc1ccc(OCc2cc(C3CC3)on2)cc1)NC(c1ccccc1)c1ccc(Cl)cc1] N-((4- chlorophenyl)(phenyl)methyl)-2-(4- ((5-cyclopropylisoxazol-3- yl)methoxy)phenyl)acetamide | ¹H NMR (400 MHz, DMSO-d₆) δ ppm 9.00-9.02 (d, 1 H), 7.38-7.40 (d, 2 H), 7.31-7.35 (m, 2 H), 7.24-7.29 (m, 5 H), 7.18-7.20 (d, 2 H), 6.92-6.94 (d, 2 H), 6.29 (s, 1 H), 6.07-6.09 (d, 1 H), 5.07 (s, 2 H), 3.47 (s, 2 H), 2.10-2.15 (m, 1 H), 1.03-1.07 (m, 2 H), 086-0.90 (m, 2 H). | LCMS-X1: 473.1 [M + H]⁺; Rₜ = 5.79 min |
| 68 | 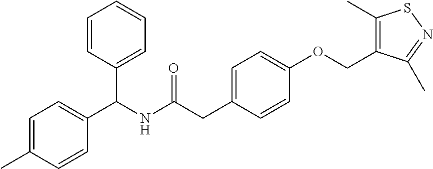 [SMILES: Cc1ccc(C(NC(=O)Cc2ccc(OCc3c(C)nsc3C)cc2)c2ccccc2)cc1] 2-(4-((3,5-dimethylisothiazol-4- yl)methoxy)phenyl)-N-(phenyl(p- tolyl)methyl)acetamide | ¹H NMR (400 MHz, CDCl₃) δ ppm 7.23-733 (m, 6 H), 7.11-7.13 (m, 4 H), 6.96-7.02 (m, 3 H), 6.22-6.24 (d, 1 H), 6.00-6.02 (d, 1 H), 4.93 (s, 2 H), 3.62 (s, 2 H), 2.54 (s, 3 H), 2.50 (s, 3 H), 2.33 (s, 3 H). | LCMS-T1: 457.0 [M + H]⁺; Rₜ = 6.74 min |
| 69 | 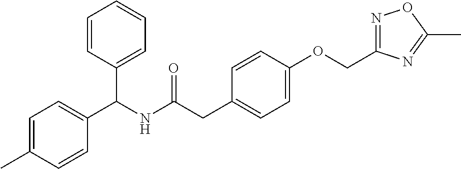 2-(4-((5-methyl-1,2,4-oxadiazol-3- yl)methoxy)phenyl)-N-(phenyl(p- tolyl)methyl)acetamide | ¹H NMR (400 MHz, DMSO-d₆) δ ppm 8.92-8.95 (d, 1 H), 7.29-7.32 (d, 2 H), 7.18-7.24 (m, 5 H), 7.10- 7.15 (m, 4 H), 6.94-6.96 (d, 2 H), 6.02-6.04 (d, 1 H), 5.19 (s, 2 H), 3.42-3.47 (s, 2 H), 2.60 (s, 3 H), 2.26 (s, 3 H). | LCMS-X1: 428.3 [M + H]⁺; Rₜ = 6.50 min |
| 70 | 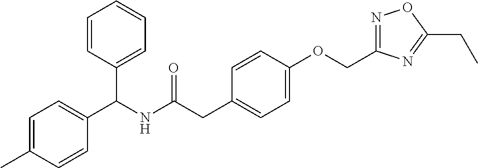 2-(4-((5-ethyl-1,2,4-oxadiazol-3- yl)methoxy)phenyl)-N-(phenyl(p- tolyl)methyl)acetamide | ¹H NMR (400 MHz, DMSO-d₆) δ ppm 8.93-8.95 (d, 1 H), 7.29-7.32 (d, 2 H), 7.19-7.24 (m, 5 H), 7.10- 7.15 (m, 4 H), 6.94-6.97 (d, 2 H), 6.02-6.04 (d, 1 H), 5.20 (s, 2 H), 3.47 (s. 2 H), 2.89-2.99 (q, 2 H), 2.26 (s, 3 H), 1.23-1.30 (t, 3 H). | LCMS-T1: = 493.1 [M + Na]⁺; Rₜ = 6.45 min |
| 71 | 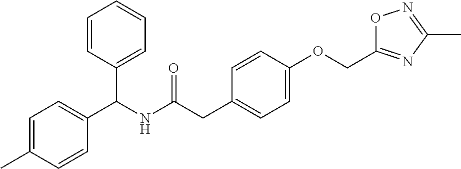 2-(4-((3-methyl-1,2,4-oxadiazol-5- yl)methoxy)phenyl)-N-(phenyl(p- tolyl)methyl)acetamide | ¹H NMR (400 MHz, DMSO-d₆) δ ppm 8.93-8.95 (d, 1 H), 7.28-7.32 (d, 2 H), 7.19-7.24 (m, 5 H), 7.10- 7.12 (m, 4 H), 6.94-6.96 (d, 2 H), 6.01-6.04 (d, 1 H), 5.44 (s, 2 H), 3.42-3.47 (s, 2 H), 2.35 (s, 3 H), 2.26 (s, 3 H). | LCMS-X1: 428.2 [M + H]⁺; Rₜ = 6.55 min |
| 72 | 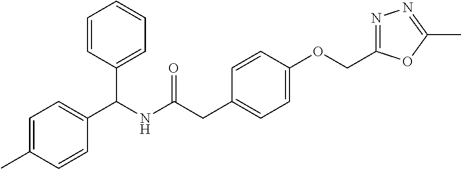 2-(4-((5-methyl-1,3,4-oxadiazol-2- yl)methoxy)phenyl)-N-(phenyl(p- tolyl)methyl)acetamide | ¹H NMR (400 MHz, DMSO-d₆) δ ppm 8.93-8.95 (d, 1 H), 7.29-7.32 (d, 2 H), 7.20-7.24 (m, 5 H), 7.10- 7.15 (m, 4 H), 6.96-6.98 (d, 2 H), 6.02-6.04 (d, 1 H), 5.32-5.34 (s, 2 H), 3.47 (s, 2 H), 2.50-2.51 (s, 3 H), 2.26 (s, 3 H). | LCMS-X1: 428.3 [M + H]⁺; Rₜ = 6.19 min |
| 73 | 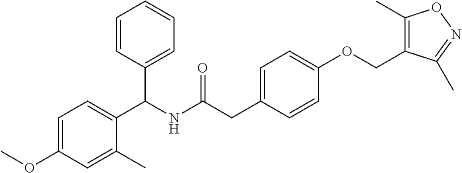 [SMILES: COc1ccc(C(NC(=O)Cc2ccc(OCc3c(C)noc3C)cc2)c2ccccc2)c(C)c1] 2-(4-((3,5-dimethylisoxazol-4- yl)methoxy)phenyl)-N-((4- methoxy-2- methylphenyl)(phenyl)methyl) acetamide | ¹H NMR (400 MHz, DMSO-d₆) δ ppm 8.80-8.82 (d, 1 H), 7.24-7.31 (m, 2 H), 7.15-7.22 (m, 5 H), 6.91- 6.97 (m, 3 H), 6.70-6.76 (m, 2 H), 6.14-6.17 (d, 1 H), 4.87 (s, 2 H), 3.71 (s, 3 H), 3.44 (s, 2 H), 2.33 (s, 3 H), 2.19 (s, 3 H), 2.17 (s, 3 H). | LCMS-T1: 493.1 [M + Na]⁺; Rₜ = 6.41 min |

Using essentially the same procedure as described in example 11, the following compounds in table 5 were made.

TABLE 5

| Ex. | Structure Name | NMR | LCMS |
|---|---|---|---|
| 74 | 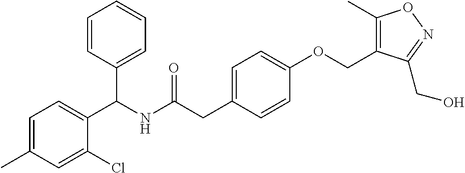 [SMILES: Cc1ccc(C(NC(=O)Cc2ccc(OCc3c(CO)noc3C)cc2)c2ccccc2)c(Cl)c1] N-((2-chloro-4- methylphenyl)(phenyl)methyl)-2- (4-((3-(hydroxymethyl)-5- methylisoxazol-4- yl)methoxy)phenyl)acetamide | ¹H NMR (400 MHz, DMSO-d₆) δ ppm 8.95-8.93 (d, 1 H), 7.27-7.19 (m, 9 H), 6.91-6.93 (d, 2 H), 6.33- 6.36 (d, 1 H), 5.41-5.44 (t, 1 H), 4.95 (s, 2 H), 4.51-4.53 (d, 2 H), 3.39-3.45 (s, 2 H), 2.40 (s, 3 H), 2.28 (s, 3 H). | LCMS-X1: 490.9 [M + H]⁺; Rₜ = 3.77 min |
| 75 | 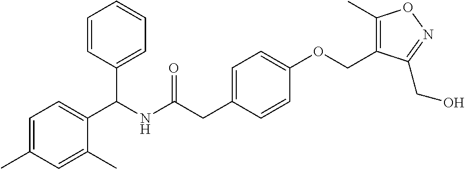 [SMILES: Cc1ccc(C(NC(=O)Cc2ccc(OCc3c(CO)noc3C)cc2)c2ccccc2)c(C)c1] N-((2,4- dimethylphenyl)(phenyl)methyl)- 2-(4-((3-(hydroxymethyl)-5- methylisoxazol-4- yl)methoxy)phenyl)acetamide | ¹H NMR (400 MHz, DMSO-d₆) δ ppm 8.82-8.84 (d, 1 H), 7.29-7.33 (m, 2 H), 7.72-7.76 (d, 1 H), 7.15- 7.19 (m, 3H), 6.97 (s, 3 H), 6.91- 6.93 (d, 2 H), 6.16-6.18 (d, 1 H), 5.41-5.44 (t, 1 H), 4.94 (s, 2 H), 4.51-4.53 (d, 2 H), 3.44 (s, 2 H), 2.40 (s, 3 H), 2.23 (s, 3 H), 2.15 (s, 3 H). | LCMS-X1: 470.0 [M + H]⁺; Rₜ = 3.80 |

Using essentially the same procedure as described in example 14, the following compounds in table 6 were made.

TABLE 6

| Ex. | Structure/Name | NMR | LCMS |
|---|---|---|---|
| 76 | 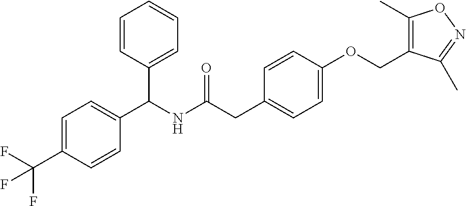 [SMILES: Cc1noc(C)c1COc1ccc(CC(=O)NC(c2ccccc2)c2ccc(C(F)(F)F)cc2)cc1] 2-(4-((3,5-dimethylisoxazol-4- yl)methoxy)phenyl)-N-(phenyl(4- (trifluoromethyl)phenyl)methyl) acetamide | ¹H NMR (500 MHz, CDCl₃) δ ppm 7.56-6.92 (m, 13H), 6.27 (d, J = 8.0 Hz, 1H), 6.03 (d, J = 8.0 Hz, 1H), 4.79 (s, 2H), 3.61 (s, 2H), 2.40 (s, 3H), 2.29 (s, 3H). | LCMS-P1: 495 [M + H]⁺; Rₜ = 1.735 min. |
| 77 | 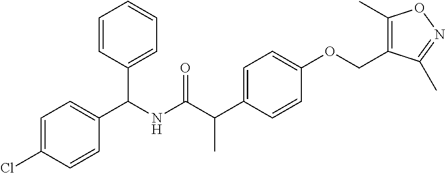 [SMILES: Cc1noc(C)c1COc1ccc(C(C)C(=O)NC(c2ccccc2)c2ccc(Cl)cc2)cc1] N-((4- chlorophenyl)(phenyl)methyl)-2- (4-((3,5-dimethylisoxazol-4- yl)methoxy)phenyl)propanamide | ¹H NMR (500 MHz, CDCl₃) δ ppm 7.31-6.90 (m, 13H), 6.16 (d, J = 9.5 Hz, 1H), 5.92 (t, J = 8.3 Hz, 1H), 4.79 (d, J = 3.5 Hz, 2H), 3.60-3.57 (m, 1H), 2.41 (d, J = 4.5 Hz, 3H), 2.29 (d, J = 4.5 Hz, 3H), 1.52-1.49 (m, 3H). | LCMS-P1: 475 [M + H]⁺; Rₜ = 1.76 min. |

Using essentially the same procedure as described in example 14, the following compounds in table 7 were made.

TABLE 7

| Ex. | Structure/Name | NMR | LCMS |
|---|---|---|---|
| 78 | 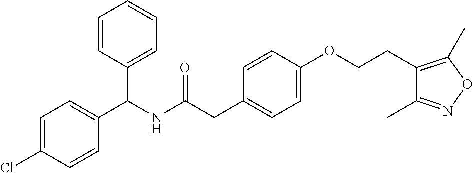 [SMILES: Cc1noc(C)c1CCOc1ccc(CC(=O)NC(c2ccccc2)c2ccc(Cl)cc2)cc1] N-((4- chlorophenyl)(phenyl)methyl)-2- (4-(2-(3,5-dimethylisoxazol-4- yl)ethoxy)phenyl)acetamide | ¹H NMR (400 MHz, DMSO-d₆) δ ppm 8.96-8.98 (d, 1 H), 7.37-7.39 (d, 2 H), 7.28-7.34 (m, 2 H), 7.23- 7.26 (m, 5 H), 7.15-7.17 (d, 2 H), 6.81-6.83 (d, 2 H), 6.07-6.09 (d, 1 H), 3.97-4.02 (t, 2 H), 3.45 (s, 2 H), 2.72-2.75 (t, 2 H), 2.32-2.33 (s, 3 H), 2.19 (s, 3 H). | LCMS-X1: 475.3.1 [M + H]⁺; Rₜ = 5.77 min |
| 79 | 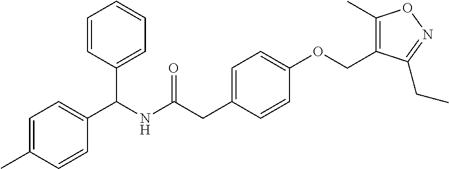 [SMILES: CCc1noc(C)c1COc1ccc(CC(=O)NC(c2ccccc2)c2ccc(C)cc2)cc1] 2-(4-((3-ethyl-5-methylisoxazol-4- yl)methoxy)phenyl)-N-(phenyl(p- tolyl)methyl)acetamide | ¹H NMR (400 MHz, DMSO-d₆) δ ppm 8.91-8.93 (d, 1 H), 7.29-7.32 (m, 2 H), 7.15-7.24 (m, 5 H), 7.10- 7.13 (m, 4 H), 6.91-6.93 (d, 2 H), 6.02-6.04 (d, 1 H), 4.87 (s, 2 H), 3.42-3.46 (s, 2 H), 2.60-2.67 (d, 2 H), 2.38 (s, 3 H), 2.26 (s, 3 H), 1.15-1.19 (s, 3 H). | LCMS-X1: 455.3 [M + H]⁺; Rₜ = 6.94 min |
| 80 | 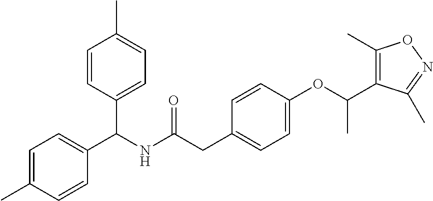 [SMILES: Cc1ccc(C(NC(=O)Cc2ccc(OC(C)c3c(C)noc3C)cc2)c2ccc(C)cc2)cc1] N-(di-p-tolylmethyl)-2-(4-(1-(3,5- dimethylisoxazol-4- yl)ethoxy)phenyl)acetamide | ¹H NMR (400 MHz, DMSO-d₆) δ ppm 8.82-8.85 (d, 1 H), 7.12-7.14 (d, 2 H), 7.08-7.10 (m, 7 H), 6.80-6.82 (d, 2 H), 5.96-5.98 (d, 1 H), 5.43-5.48 (q, 1 H), 3.37 (s, 2 H), 2.50 (s, 3 H), 2.37 (s, 6 H), 2.15 (s, 3 H), 1.52-1.53 (d, 3 H). | LCMS-X1: 469.3 [M + H]⁺; Rₜ = 7.16 min |
| 81 | 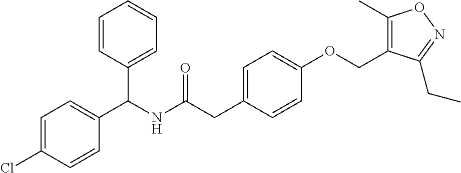 [SMILES: CCc1noc(C)c1COc1ccc(CC(=O)NC(c2ccccc2)c2ccc(Cl)cc2)cc1] N-((4- chlorophenyl)(phenyl)methyl)-2- (4-((3-ethyl-5-methylisoxazol-4- yl)methoxy)phenyl)acetamide | ¹H NMR (400 MHz, DMSO-d₆) δ ppm 9.00-9.25 (d, 1 H), 8.90 (s, 1 H), 7.38-7.40, (d, 2 H), 7.33-7.35 (d, 2 H), 7.18-7.31 (m, 5 H), 6.91- 6.9.3 (d, 2 H), 6.07-6.09 (d, 1 H), 5.87 (s, 2 H), 3.45-3.50 (t, 2 H), 2.59-2.65 (m, 2 H), 2.50-2.51 (t, 3 H), 1.15-1.19 (t, 3 H). | LCMS-X1: 475.1 [M + H]⁺; Rₜ = 3.43 min |
| 82 | 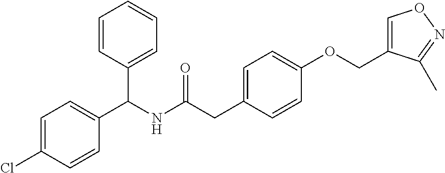 [SMILES: Cc1nocc1COc1ccc(CC(=O)NC(c2ccccc2)c2ccc(Cl)cc2)cc1] N-((4- chlorophenyl)(phenyl)methyl)-2- (4-((3-methylisoxazol-4- yl)methoxy)phenyl)acetamide | ¹H NMR (400 MHz, DMSO-d₆) δ ppm 8.99-9.01 (d, 1 H), 8.91 (s, 1 H), 7.38-7.40 (d, 2 H), 7.24-7.35 (m, 8 H), 7.18-7.20 (d, 2 H), 6.93- 6.95 (d, 1 H), 4.96 (s, 2 H), 3.47 (s, 2 H), 2.33 (s, 3 H). | LCMS-X1: 447.2 [M + H]⁺; Rₜ = 7.41 min |
| 83 | 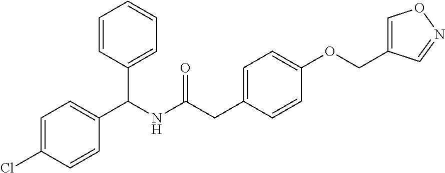 [SMILES: O=C(Cc1ccc(OCc2cnoc2)cc1)NC(c1ccccc1)c1ccc(Cl)cc1] N-((4- chlorophenyl)(phenyl)methyl)-2- (4-(isoxazol-4- ylmethoxy)phenyl)acetamide | ¹H NMR (400 MHz, DMSO-d₆) δ ppm 9.06 (s, 1 H), 8.99-9.01 (d, 1 H), 8.73 (s, 1 H), 7.38-7.40 (d, 2 H), 7.29-7.35 (m, 3 H), 7.24-7.27 (m, 4 H), 7.18-7.20 (d 2 H), 6.92- 6.94 (d, 1 H), 6.07-6.09 (d, 2 H), 4.99 (s, 2 H), 3.41-3.47 (d, 2 H). | LCMS-T1: 433.0 [M + H]⁺; Rₜ = 6.43 min |
| 84 | 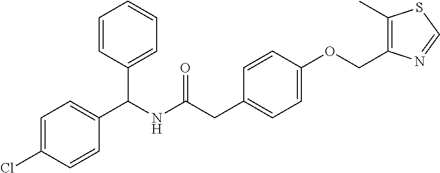 [SMILES: Cc1scnc1COc1ccc(CC(=O)NC(c2ccccc2)c2ccc(Cl)cc2)cc1] N-((4- chlorophenyl)(phenyl)methyl)-2- (4-((5-methylthiazol-4- yl)methoxy)phenyl)acetamide | ¹H NMR (400 MHz, CDCl₃) δ ppm 8.98-9.00 (d, 1 H), 8.88 (s, 1 H), 7.38-7.40, (d, 2 H), 7.33-7.35 (d, 2 H), 7.24-7.29 (m 5 H), 7.17- 7.19 (d, 2 H), 6.94-6.96 (d, 2 H), 6.08-6.10 (d, 1 H), 5.09 (s, 2 H), 3.47 (s, 2 H), 2.47 (s, 3 H). | LCMS-X1: 463.1 [M + H]⁺; Rₜ = 5.64 min |

Using essentially the same procedure as described in example 20, the following compound in table 8 was made.

TABLE 8

| Ex. | Structure/Name | NMR | LCMS |
|---|---|---|---|
| 85 | 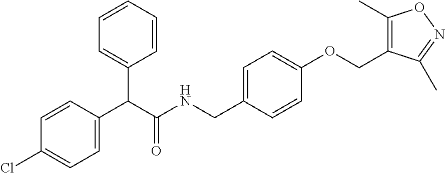 [SMILES: Cc1noc(C)c1COc1ccc(CNC(=O)C(c2ccccc2)c2ccc(Cl)cc2)cc1] 2-(4-chlorophenyl)-N-(4- ((3,5-dimethylisoxazol-4- yl)methoxy)benzyl)-2- phenylacetamide | ¹H NMR (400 MHz, DMSO-d₆) δ ppm 8.73-8.76 (t, 1 H), 7.36-7.39 (d, 2 H), 7.29-7.34 (m, 6 H), 7.23- 6.26 (m, 1 H), 7.13-7.15 (d, 2 H), 6.91-6.94 (d, 2 H), 4.99 (s, 1 H), 4.88 (s, 2 H), 4.22-4.24 (d, 2 H), 2.38 (s, 3 H), 2.19 (s, 3H). | LCMS-X1: 461.4 [M + H]⁺; Rₜ = 6.94 min |

### Example 86

### 2-(1-(3,5-Dimethylisoxazol-4-yl)-2-(4-(2-(((2,4-dimethylphenyl)(phenyl)methyl)amino)-2-oxoethyl)phenoxy)ethoxy)-2-methylpropanoic acid

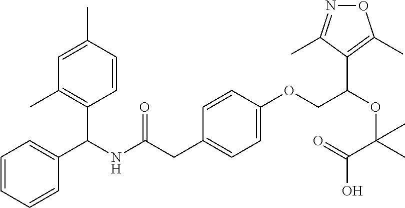 [SMILES: Cc1ccc(C(NC(=O)Cc2ccc(OCC(OC(C)(C)C(=O)O)c3c(C)noc3C)cc2)c2ccccc2)c(C)c1]

### (a) 2-(4-(2-(3,5-Dimethylisoxazol-4-yl)-2-oxoethoxy)phenyl)-N-((2,4-dimethylphenyl)(phenyl)methyl)acetamide

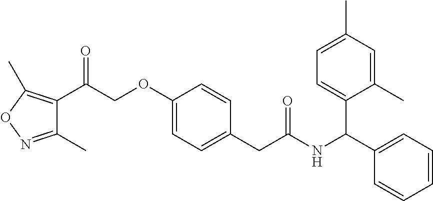 [SMILES: Cc1ccc(C(NC(=O)Cc2ccc(OCC(=O)c3c(C)noc3C)cc2)c2ccccc2)c(C)c1]

2-Bromo-1-(3,5-dimethylisoxazol-4-yl)ethanone (1.3 g, 0.006 mmol) and K₂CO₃ (1.1 g, 0.008 mol) were added to a solution of N-((2,4-dimethylphenyl)(phenyl)methyl)-2-(4-hydroxyphenyl)acetamide (1.5 g, 0.004 mmol) in MeCN (25 mL) under N₂. The mixture was stirred at 70° C. for 8 h. Water (50 mL) was then added to reaction mixture, and the mixture was extracted with ethyl acetate (3×50 mL). The combined extracts were washed with brine (3×15 mL), dried over Na₂SO₄, and concentrated under reduced pressure to yield a yellow oil. The oil was purified by silica gel chromatography using 50% EtOAc in PE to give white solid (1.2 g, yield 60%). LC-MSA022: 505.1[M+H]⁺; RT=1.84 min, purity 96.54% (254 nm).

### (b) 2-(4-(2-(3,5-dimethylisoxazol-4-yl)-2-hydroxyethoxy)phenyl)-N-((2,4-dimethylphenyl)(phenyl)methyl)acetamide

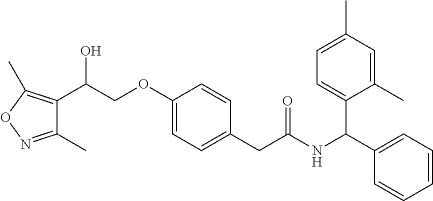 [SMILES: Cc1ccc(C(NC(=O)Cc2ccc(OCC(O)c3c(C)noc3C)cc2)c2ccccc2)c(C)c1]

NaBH₄ (105 mg, 2.75 mmol) was added to a solution of 2-(4-(2-(3,5-dimethylisoxazol-4-yl)-2-oxoethoxy)phenyl)-N-((2,4-dimethylphenyl)(phenyl)methyl)acetamide (1.2 g, 2.5 mmol) in MeOH (18 mL). The mixture was stirred at rt for 0.5 h. After completion of the reaction, water (30 mL) was added and the mixture was extracted with ethyl acetate (3×30 mL). The combined extracts were washed with brine (3×15 mL), dried over Na₂SO₄, and concentrated under reduced pressure to yield a white solid (680 mg, yield 56%), which was used in the next step without further purification. LC-MSA011: 485.2 [M+H]⁺; RT=2.00 min, purity 94.20% (254 nm).

### (c) Methyl 2-(1-(3,5-dimethylisoxazol-4-yl)-2-(4-(2-(((2,4-dimethylphenyl)(phenyl)methyl)amino)-2-oxoethyl)phenoxy)ethoxy)-2-methylpropanoate

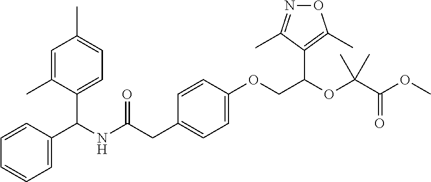 [SMILES: COC(=O)C(C)(C)OC(COc1ccc(CC(=O)NC(c2ccccc2)c2ccc(C)cc2C)cc1)c1c(C)noc1C]

Methyl 2-bromo-2-methylpropanoate (42 mg, 0.23 mmol) and Cs2CO3 (147 mg, 0.45 mmol) were added to a solution of 2-(4-(2-(3,5-dimethylisoxazol-4-yl)-2-hydroxyethoxy)phenyl)-N-((2,4-dimethylphenyl)(phenyl)methyl)acetamide (75 mg, 0.15 mmol) in CH3CN (15 mL). The mixture was stirred at 70° C. for 8 h. Water (30 mL) was added to reaction mixture and the mixture was extracted with ethyl acetate (3×30 mL). The combined extracts were washed with brine (3×15 mL), dried over Na₂SO₄, and concentrated under reduced pressure to give a yellow oil. The oil was purified by silica gel Prep-TLC using 50% EtOAc in petroleum ether to give methyl 2-(1-(3,5-dimethylisoxazol-4-yl)-2-(4-(2-(((2,4-dimethylphenyl)(phenyl)methyl)amino)-2-oxoethyl)phenoxy)ethoxy)-2-methylpropanoate (100 mg). LCMS-A026: 585.3.2[M+H]⁺; Rt=2.00 min.

### (d) 2-(1-(3,5-Dimethylisoxazol-4-yl)-2-(4-(2-(((2,4-dimethylphenyl)(phenyl)methyl)amino)-2-oxoethyl)phenoxy)ethoxy)-2-methylpropanoic acid

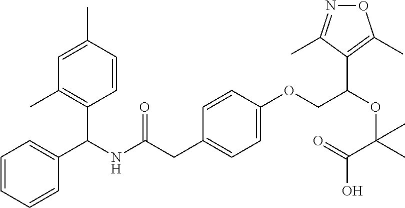 [SMILES: Cc1ccc(C(NC(=O)Cc2ccc(OCC(OC(C)(C)C(=O)O)c3c(C)noc3C)cc2)c2ccccc2)c(C)c1]

A solution of LiOH (6 mg, 0.24 mmol) in water (3 mL) was added to the suspension of methyl 2-(1-(3,5-dimethylisoxazol-4-yl)-2-(4-(2-(((2,4-dimethylphenyl)(phenyl)methyl)amino)-2-oxoethyl)phenoxy)ethoxy)-2-methylpropanoate (68 mg, 0.12 mmol) in THF (5 mL). After stirring at rt for 2 h, the reaction mixture was evaporated under reduced pressure to remove the excess THF. The reaction was acidified to pH˜7 with 1M HCl solution and extracted with ethyl acetate (3×50 mL), and dried over Na₂SO₄. After removal of the solvent, the residue was purified by reverse phase HPLC using water/acetonitrile with 0.05% TFA to obtain 2-(1-(3,5-dimethylisoxazol-4-yl)-2-(4-(2-(((2,4-dimethylphenyl)(phenyl)methyl)amino)-2-oxoethyl)phenoxy)ethoxy)-2-methylpropanoic acid (11 mg, yield: 16%). LCMS-A024: 571.3 [M+H]⁺; Rt=1.74 min. ¹H NMR (MeOD, 400 MHz): δ 7.31-7.20 (m, 5H), 7.13 (d, J=7.2 Hz, 2H), 6.99 (s, 1H), 6.94 (s, 2H), 6.84 (d, J=8.8 Hz, 2H), 6.29-6.27 (m, 1H), 4.97-4.89 (m, 1H), 4.36-4.31 (m, 1H), 4.08-4.05 (m, 1H), 3.50 (s, 2H), 2.35 (s, 3H), 2.29 (s, 3H), 2.28 (s, 3H), 2.17 (s, 3H), 1.46 (s, 3H), 1.35 (s, 3H).

### Examples 87 and 88

### (S)—N-((4-chloro-2-methylphenyl)(4-chlorophenyl)methyl)-2-(4-((3,5-dimethylisoxazol-4-yl)methoxy)phenyl)acetamide and (R)—N-((4-chloro-2-methylphenyl)(4-chlorophenyl)methyl)-2-(4-((3,5-dimethylisoxazol-4-yl)methoxy)phenyl)acetamide

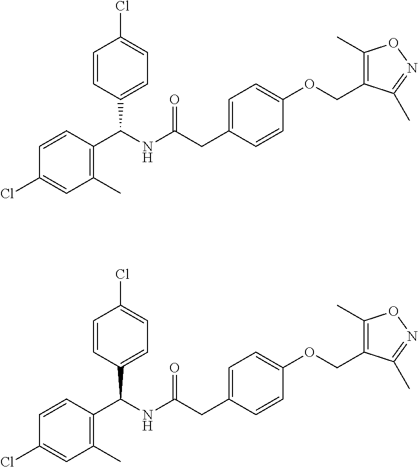 [SMILES: Cc1cc(Cl)ccc1[C@@H](NC(=O)Cc1ccc(OCc2c(C)noc2C)cc1)c1ccc(Cl)cc1 ; Cc1cc(Cl)ccc1[C@H](NC(=O)Cc1ccc(OCc2c(C)noc2C)cc1)c1ccc(Cl)cc1]

N-((4-chloro-2-methylphenyl)(4-chlorophenyl)methyl)-2-(4-((3,5-dimethylisoxazol-4-yl)methoxy)phenyl)acetamide (180 mg) was resolved using the following method:

Instrument: Thar SFC Prep 80 (Thar Technologies, Waters); Column: ChiralCell OJ-H, 20 mm I.D.×250 mm Length, 5 μm (Daicel Chemical Industries Co., Ltd); Column Temperature: 35° C.; Mobile Phase: CO2/MeOH/DEA=70/30/0.1; Flow rate: 50 g/min; Back Pressure: 100 Bar; Wavelength: 214 nm; Cycle time: 3.0 min; Injection Volume: 0.6 mL; Load per injection: 6 mg; Feed solution: 180 mg dissolved in 18 mL MeOH
Said resolution yielded 2 enantiomers:
peak 1, 75 mg, RT 6.4 min, 95.0% de, 97.5% purity
peak 2, 58 mg, RT 7.7 min, 100% de, 100% purity

### Example 89

### 4-((4-(2-(((4-chloro-2-methylphenyl)(phenyl)methyl)amino)-2-oxoethyl)phenoxy)methyl)-5-methylisoxazole-3-carboxylic acid

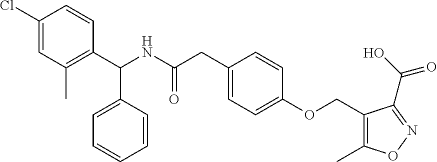 [SMILES: Cc1cc(Cl)ccc1C(NC(=O)Cc1ccc(OCc2c(C(=O)O)noc2C)cc1)c1ccccc1]

### (a) (4-chloro-2-methylphenyl)magnesium bromide

This compound was synthesized from 1-bromo-4-chloro-2-methylbenzene essentially as described in example 1 (d) to give the title compound which was used in the next step without further purification.

### (b) (4-chloro-2-methylphenyl)(phenyl)methanamine

This compound was synthesized from benzonitrile and (4-chloro-2-methylphenyl)magnesium bromide essentially as described in example 1 (e) to give the title compound (17.1 g, 64% yield), which was used in the next step without further purification. LC-MS: 215.0 [M-NH2]⁺; Rt=1.261 min.

### (c) N-((4-chloro-2-methylphenyl)(phenyl)methyl)-2-(4-hydroxyphenyl)acetamide

This compound was synthesized from (4-chloro-2-methylphenyl)(phenyl)methanamine hydrochloride and 2-(4-hydroxyphenyl)acetic acid essentially as described in example 4 (b) to give the title compound (650 mg, Yield: 63.6%). LC-MS: m/z 366 [M+H]; Rt=1.93 min.

### (d) methyl 4-((4-(2-(((4-chloro-2-methylphenyl)(phenyl)methyl)amino)-2-oxoethyl)phenoxy)methyl)-5-methylisoxazole-3-carboxylate

This compound was synthesized from N-((4-chloro-2-methylphenyl)(phenyl)methyl)-2-(4-hydroxyphenyl)acetamide and methyl 4-(chloromethyl)-5-methylisoxazole-3-carboxylate essentially as described in example 1 (b) to give the title compound (210 mg, Yield: 70%). LC-MS: m/z 519 [M+H]⁺; Rt=2.06 min.

### (e) 4-((4-(2-(((4-chloro-2-methylphenyl)(phenyl)methyl)amino)-2-oxoethyl)phenoxy)methyl)-5-methylisoxazole-3-carboxylic acid

NaOH (33 mg, 0.82 mmol) was added to a solution of methyl 4-((4-(2-(((4-chloro-2-methylphenyl)(phenyl)methyl)amino)-2-oxoethyl)phenoxy)methyl)-5-methylisoxazole-3-carboxylate (210 mg, 0.41 mmol) in THF (8 mL) and water (2 mL). The mixture was stirred at rt overnight. Then water (10 mL) was added to the mixture, and then AcOH was used to adjust the aqueous phase to pH˜6. The mixture was then extracted with ethyl acetate (3×30 mL). The combined extracts were washed with brine (3×15 mL), dried over Na₂SO₄, and concentrated under reduced pressure to give a yellow oil. The residue was purified by column chromatography on silica gel (EA:PE=1:2) to provide 4-((4-(2-(((4-chloro-2-methylphenyl)(phenyl)methyl)amino)-2-oxoethyl)phenoxy)methyl)-5-methylisoxazole-3-carboxylic acid (30 mg, Yield: 14.6%). LC-MS: 505[M+H]⁺; Rt=1.71 min. ¹H NMR (MEOD, 400 MHz): 7.30-7.33 (m, 3H), 7.13-7.30 (m, 6H), 7.03-7.29 (m, 5H), 7.03 (m, 3H), 6.94 (m, 3H), 6.27 (s, 1H), 5.18 (s, 2H), 3.51 (s, 2H), 2.45 (s, 3H), 2.20 (s, 3H).

### Example 90

### 2-((3,5-dimethylisoxazol-4-yl)methoxy)-5-(2-(((2,4-dimethylphenyl)(phenyl)methyl)amino)-2-oxoethyl)benzoic acid

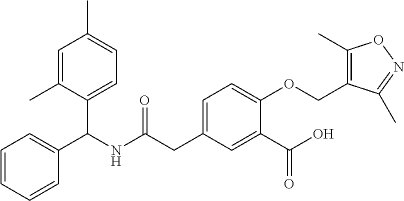 [SMILES: Cc1ccc(C(NC(=O)Cc2ccc(OCc3c(C)noc3C)c(C(=O)O)c2)c2ccccc2)c(C)c1]

This compound was synthesized from methyl 2-((3,5-dimethylisoxazol-4-yl)methoxy)-5-(2-(((2,4-dimethylphenyl)(phenyl)methyl)amino)-2-oxoethyl)benzoate essentially as described in example 1 (c) (18 mg, 78%). LCMS-P1: 521.2 [M+Na]⁺; Rₜ=1.676 min. ¹H NMR (400 MHz, DMSO-d₆) δ ppm 12.69 (s, 1H), 8.93 (d, J=8.4 Hz, 1H), 7.64 (d, J=2.0 Hz, 1H), 7.46-7.25 (m, 7H), 7.05-7.00 (m, 3H), 6.23 (d, J=8.4 Hz, 1H), 5.00 (s, 2H), 3.55 (s, 2H), 2.45 (s, 3H), 2.30 (s, 3H), 2.29 (s, 3H), 2.21 (s, 3H).

### Example 91

### 2-(4-((3,5-dimethylisoxazol-4-yl)methoxy)phenyl)-1-(1-phenyl-3,4-dihydroisoquinolin-2(1H)-yl)ethanone

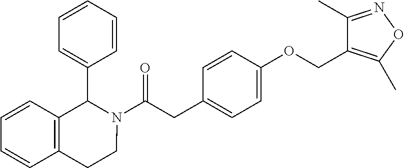 [SMILES: Cc1noc(C)c1COc1ccc(CC(=O)N2CCc3ccccc3C2c2ccccc2)cc1]

This compound was synthesized from 1-phenyl-1,2,3,4-tetrahydroisoquinoline hydrochloride and 2-(4-((3,5-dimethylisoxazol-4-yl)methoxy)phenyl)acetic acid essentially as described in example 4 (b) (136 mg, 63% yield). LCMS: 453.2 [M+H]; Rₜ=6.795 min

### Example 92

### N-((4-chloro-2-methylphenyl)(4-chlorophenyl)methyl)-2-(4-(2-(3,5-dimethylisoxazol-4-yl)ethoxy)phenyl)acetamide

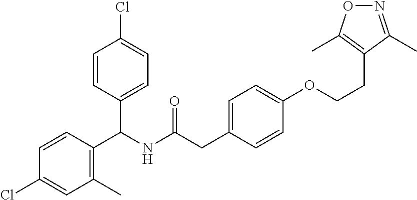 [SMILES: Cc1cc(Cl)ccc1C(NC(=O)Cc1ccc(OCCc2c(C)noc2C)cc1)c1ccc(Cl)cc1]

This compound was synthesized from (4-chloro-2-methylphenyl)(4-chlorophenyl)methanamine and 2-(4-((3,5-dimethylisoxazol-4-yl)methoxy)phenyl)acetic acid essentially as described in example 20 (b) (0.017 g, 17.89%) ¹H NMR (400 MHz, DMSO) δ 8.88-8.90 (d, 1H), 7.34-7.41 (m, 2H), 7.24-7.27 (t, 2H), 7.13-7.28 (m, 6H), 7.03-7.05 (d, 1H), 6.81-6.84 (d, 2H), 6.15-6.17 (d, 1H), 3.97-4.01 (t, 2H), 3.33-3.38 (d, 1H), 2.72-2.76 (m, 2H), 2.32-2.36 (d, 3H), 2.18-2.20 (d, 3H), LCMS purity: 94.52%.

### Example 93

### 2-(4-(2-(3,5-dimethylisoxazol-4-yl)ethoxy)phenyl)-N-((2,4-dimethylphenyl)(phenyl)methyl)acetamide

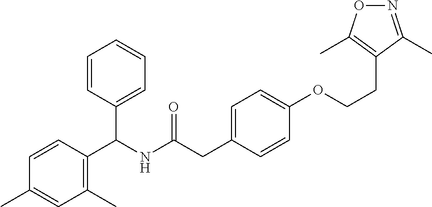

This compound was synthesized from (2,4-dimethylphenyl)(phenyl)methanamine and 2-(4-(2-(3,5-dimethylisoxazol-4-yl)ethoxy)phenyl)acetic acid essentially as described in example 20 (b) (7.0 mg, 8.23%). ¹H NMR (400 MHz, DMSO) δ 8.81-8.80 (d, 1H), 7.29-7.32 (t, 2H), 7.22-7.25 (t, 1H), 7.15-7.17 (m, 3H), 6.94-6.97 (t 2H), 6.81-6.83 (d, 2H), 6.16-6.18 (d, 1H), 3.97-4.00 (t, 2H), 3.39 (s, 2H), 2.72-2.76 (t, 2H), 2.32 (s, 3H), 2.20 (s, 6H), 2.14 (s, 3H), LCMS purity: 97.58%.

### Example 94

### N-(bis(2-chlorophenyl)methyl)-2-(4-((3,5-dimethylisoxazol-4-yl)methoxy)phenyl)acetamide

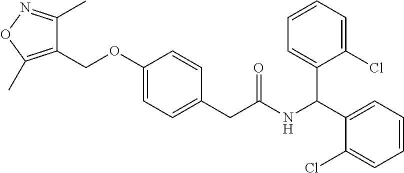 [SMILES: Cc1noc(C)c1COc1ccc(CC(=O)NC(c2ccccc2Cl)c2ccccc2Cl)cc1]

This compound was synthesized from bis(2-chlorophenyl)methanamine and 2-(4-((3,5-dimethylisoxazol-4-yl)methoxy)phenyl)acetic acid essentially as described in example 20 (b) (40.2 mg, 21.72%). ¹H NMR (400 MHz, DMSO) δ 7.44-7.45 (d, 2H), 7.33-7.35 (d, 1H), 7.31-7.35 (d, 1H), 7.21-7.30 (m, 4H), 7.07-7.09 (dd, 2H), 6.84-6.86 (d, 2H), 6.72-6.73 (d, 1H), 4.05-4.08 (t, 2H), 3.53 (s, 2H), 2.85-2.88 (t, 2H), 2.38 (s, 3H), 2.28 (s, 3H), LCMS purity 97.72%.

### Example 95

### 2-(4-((3-ethyl-5-methylisoxazol-4-yl)methoxy)phenyl)-N-(phenyl(o-tolyl)methyl)acetamide

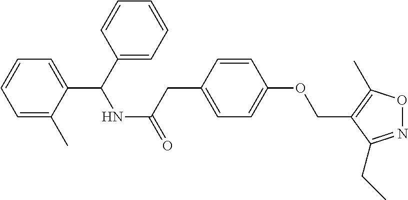 [SMILES: CCc1noc(C)c1COc1ccc(CC(=O)NC(c2ccccc2)c2ccccc2C)cc1]

Triphenylphosphine (0.189 g, 0.725 mmol) and (3-ethyl-5-methylisoxazol-4-yl)methanol (0.102 g, 0.725 mmol) were added to a solution of 2-(4-hydroxyphenyl)-N-(phenyl(o-tolyl)methyl)acetamide (0.20 g, 0.604 mmol) in dry THF (1.0 mL). The reaction mixture was then cooled in a 33-KHz sonicating bath and sonicated it for 3 min giving a clear solution. While sonicating, diisopropylazodicarboxylate (0.146 mL, 0.725 mmol) was added drop-wise to the reaction mixture during 2 min. The reaction mixture was then sonicated for an additional 15 min, and then THF was removed under reduced pressure. The resulting residue was purified by silica gel chromatography (0.2% methanol/CH₂Cl₂) and then re-purified with prep HPLC to provide 2-(4-((3-ethyl-5-methylisoxazol-4-yl)methoxy)phenyl)-N-(phenyl(o-tolyl)methyl)acetamide (95 mg, 34.67%). ¹H NMR (400 MHz, DMSO) δ 8.89-8.91 (d, 1H), 7.30-7.34 (d, 2H), 7.23-7.27 (m, 1H), 7.18-7.20 (m, 6H), 7.10-7.17 (m, 1H), 6.91-6.93 (d, 2H), 6.21-6.23 (d, 1H), 4.87 (s, 2H), 3.45 (s, 2H), 2.60-2.64 (q, 2H), 2.39 (s, 3H), 2.19 (s, 3H), 1.15-1.19 (t, 3H). MS (ESI+) m/z 455.30 (M+H), LCMS purity: 99.93%.

### Biological Data

As stated above, the compounds according to Formula (I) are RORγ modulators, and are useful in the treatment of diseases mediated by RORγ. The biological activities of the compounds according to Formula (I) can be determined using any suitable assay for determining the activity of a candidate compound as a RORγ modulator, as well as tissue and in vivo models.

### Dual Fluorescence Energy Transfer (FRET) Assay

This assay is based on the knowledge that nuclear receptors interact with cofactors (transcription factors) in a ligand dependent manner. RORγ is a typical nuclear receptor in that it has an AF2 domain in the ligand binding domain (LBD) which interacts with co-activators. The sites of interaction have been mapped to the LXXLL motifs in the co-activator SRCl(2) sequences. Short peptide sequences containing the LXXLL motif mimic the behavior of full-length co-activator.

The assay measures ligand-mediated interaction of the co-activator peptide with the purified bacterial-expressed RORγ ligand binding domain (RORγ-LBD) to indirectly assess ligand binding. RORγ has a basal level of interaction with the co-activator SRCl(2) in the absence of ligand, thus it is possible to find ligands that inhibit or enhance the RORγ/SRCl(2) interaction.

### Materials

### Generation of RORγ-LBD Bacterial Expression Plasmid

Human RORγ Ligand Binding Domain (RORγ-LBD) was expressed in E. coli strain BL21(DE3) as an amino-terminal polyhistidine tagged fusion protein. DNA encoding this recombinant protein was sub-cloned into a modified pET21a expression vector (Novagen). A modified polyhistidine tag (MKKHHHHHHLVPRGS) (SEQ ID No: 1) was fused in frame to residues 263-518 of the human RORγ sequence.

### Protein Purification

Approximately 50 g E. coli cell pellet was resuspended in 300 mL of lysis buffer (30 mM imidazole pH 7.0 and 150 mM NaCl). Cells were lysed by sonication and cell debris was removed by centrifugation for 30 minutes at 20,000 g at 4° C. The cleared supernatant was filtered through a 0.45 μM cellulose acetate membrane filter. The clarified lysate was loaded onto a column (XK-26) packed with ProBond Nickel Chelating resin (InVitrogen), pre-equilibrated with 30 mM imidazole pH 7.0 and 150 mM NaCl. After washing to baseline absorbance with the equilibration buffer, the column was developed with a gradient from 30 to 500 mM imidazole pH 7.0. Column fractions containing the RORγ-LBD protein were pooled and concentrated to a volume of 5 mL. The concentrated protein was loaded onto a Superdex 200 column pre-equilibrated with 20 mM Tris-Cl pH 7.2 and 200 mM NaCl. The fractions containing the desired RORγ-LBD protein were pooled together.

### Protein Biotinylation

Purified RORγ-LBD was buffer exchanged by exhaustive dialysis [3 changes of at least 20 volumes (>8000×)] against PBS [100 mM NaPhosphate, pH 8 and 150 mM NaCl]. The concentration of RORγ-LBD was approximately 30 μM in PBS. Five-fold molar excess of NHS-LC-Biotin (Pierce) was added in a minimal volume of PBS. This solution was incubated with occasional gentle mixing for 60 minutes at ambient rt. The modified RORγ-LBD was dialyzed against 2 buffer changes—TBS pH 8.0 containing 5 mM DTT, 2 mM EDTA and 2% sucrose—each at least 20 times of the volume. The modified protein was distributed into aliquots, frozen on dry ice and stored at −80° C. The biotinylated RORγ-LBD was subjected to mass spectrometric analysis to reveal the extent of modification by the biotinylation reagent. In general, approximately 95% of the protein had at least a single site of biotinylation and the overall extent of biotinylation followed a normal distribution of multiple sites ranged from one to five.

A biotinylated peptide corresponding to amino acid 676 to 700 (CPSSHSSLTERHKILHRLLQEGSPS) (SEQ ID No: 2) of the co-activator steroid receptor coactivator SRCl(2) was generated using similar method.

### Assay

Preparation of Europium labeled SRCl(2) peptide: biotinylated SRCl(2) solution was prepared by adding an appropriate amount of biotinylated SRCl(2) from the 100 μM stock solution to a buffer containing 10 mM of freshly added DTT from solid to give a final concentration of 40 nM. An appropriate amount of Europium labeled Streptavidin was then added to the biotinylated SRCl(2) solution in a tube to give a final concentration of 10 nM. The tube was inverted gently and incubated for 15 minutes at rt. Twenty-fold excess biotin from the 10 mM stock solution was added and the tube was inverted gently and incubated for 10 minutes at rt.

Preparation of APC labeled RORγ-LBD: biotinylated RORγ-LBD solution was prepared by adding an appropriate amount of biotinylated RORγ-LBD from the stock solution to a buffer containing 10 mM of freshly added DTT from solid to give a final concentration of 40 nM. An appropriate amount of APC labeled Streptavidin was then added to the biotinylated RORγ-LBD solution in a tube to give a final concentration of 20 nM. The tube was inverted gently and incubated for 15 minutes at rt. Twenty-fold excess biotin from the 10 mM stock solution was then added and the tube was inverted gently and incubated for 10 minutes at rt.

Equal volumes of the above-described Europium labeled SRCl(2) peptide and the APC labeled RORγ-LBD were gently mixed together to give 20 nM RORγ-LBD, 10 nM APC-Strepavidin, 20 nM SRCl(2) and 5 nM Europium-Streptavidin. The reaction mixtures were incubated for 5 minutes. Using a Thermo Combi Multidrop 384 stacker unit, 25 L of the reaction mixtures per well was added to the 384-well assay plates containing 1 μL of test compound per well in 100% DMSO. The plates were incubated for 1 hour and then read on ViewLux in Lance mode for EU/APC.

Results

The compounds of Examples 1-95 were tested in the dual FRET assay described above, and were found to have a pIC₅₀ between 4.4 and 9.

1. A compound according to Formula (I): 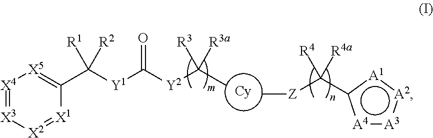
wherein:
m is 0, 1, or 2;
n is 0, 1, 2, or 3;
X¹, X², X³, X⁴, and X⁵ are each independently selected from N, N⁺—O⁻, CH, and CR⁵, wherein 0-3 of X¹, X², X³, X⁴, and X⁵ are N or N⁺—O⁻ and 1-3 of X¹, X², X³, X⁴, and X⁵ are CR⁵; provided that when zero of X¹, X², X⁴, and X⁵ are N or N⁺—O⁻ and X³ is CR⁵, 1-2 of X¹, X², X⁴, and X⁵ are CR⁵;
one of Y¹ and Y² is O or NR⁸ and the other is a bond;
or X¹ is CR⁵, Y¹ is NR⁸, Y² is a bond, and R⁵ and R⁸ taken together with the atoms to which they are attached form a five to seven membered ring, optionally containing an additional heteroatom selected from oxygen, nitrogen, and sulfur, which ring is optionally substituted by (C₁-C₄)alkyl;
Cy is (C₃-C₈)cycloalkyl, heterocycloalkyl, phenyl, or 5- or 6-membered heteroaryl, each of which is optionally substituted one, two, or three times, independently, by (C₁-C₆)alkyl, (C₁-C₆)haloalkyl, (C₃-C₆)cycloalkyl, halogen, oxo, cyano, hydroxyl, hydroxy(C₁-C₆)alkyl, (C₁-C₆)alkoxy, —((C₀-C₃)alkyl)NHCO₂R⁷, —((C₀-C₃)alkyl)N((C₁-C₄)alkyl)C₂R⁷, —((C₀-C₃)alkyl)NHC(O)R⁷, —((C₀-C₃)alkyl)N((C₁-C₄)alkyl)C(O)R⁷, —((C₀-C₃)alkyl)CO₂R⁷, —((C₀-C₃)alkyl)CONR⁷R⁸, —((C₀-C₃)alkyl)C(O)R⁷, (C₁-C₄)alkoxy(C₁-C₆)alkyl, amino(C₁-C₆)alkyl, ((C₁-C₄)alkyl)((C₁-C₄)alkyl)amino(C₁-C₆)alkyl, (C₁-C₄)alkylamino(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, or heterocycloalkyl;
Z is O, S, SO₂, C═O, NR⁶, or a bond;
A¹, A², A³, and A⁴ are each independently selected from N, NR⁶, O, S, CH, and CR¹⁰, wherein one of A¹, A², A³, and A⁴ is NR⁶, O, or S, 0-2 of A¹, A², A³, and A⁴ are CR¹⁰, and 0-3 of A¹, A², A³, and A⁴ are CH or N;
R¹ is (C₃-C₆)alkyl, (C₃-C₆)haloalkyl, (C₃-C₈)cycloalkyl, (C₃-C₆)alkoxy, (C₁-C₆)alkoxy(C₁-C₂)alkyl, aryl, heteroaryl, aryl(C₁-C₆)alkyl, heteroaryl(C₁-C₆)alkyl, or heterocycloalkyl, each of which is optionally substituted one, two, or three times, independently, by R⁵;
R² is hydrogen, (C₁-C₆)alkyl, or (C₁-C₆)haloalkyl;
or R¹ and R² taken together with the carbon atom to which they are attached form a three to eight membered ring, optionally containing a heteroatom selected from oxygen, nitrogen, and sulfur, which ring is optionally substituted one, two, or three times, independently, by R⁵;
R³ and R^{3a} are each independently hydrogen, hydroxyl, (C₁-C₆)alkyl, (C₁-C₆)haloalkyl, halogen, (C₁-C₆)alkoxy, amino, (C₁-C₄)alkylamino, or ((C₁-C₄)alkyl)((C₁-C₄)alkyl)amino;
each R⁴ is independently selected from hydrogen, halogen, (C₁-C₆)alkyl, (C₁-C₆)haloalkyl, —CO₂R⁷, —CONR⁷R⁸, —OR⁹, and —NR⁸R⁹, wherein said (C₁-C₆)alkyl or (C₁-C₆)haloalkyl is optionally substituted by hydroxyl, —OR⁹, —CO₂R⁷, —CONR⁷R⁸, or —NR⁸R⁹;
each R^{4a} is independently selected from hydrogen, halogen, hydroxyl, amino, and (C₁-C₆)alkyl;
or R⁴ and R^{4a} taken together with the carbon atom to which they are attached form a three to eight membered ring, optionally containing a heteroatom selected from oxygen, nitrogen, and sulfur, which ring is optionally substituted by cyano, (C₁-C₄)alkyl, (C₁-C₄)haloalkyl, (C₃-C₆)cycloalkyl, —CO₂R⁷, —CONR⁷R⁸, hydroxyl, hydroxy(C₁-C₆)alkyl, (C₁-C₄)alkoxy, (C₁-C₄)alkoxy(C₁-C₆)alkyl, amino, (C₁-C₄)alkylamino, ((C₁-C₄)alkyl)((C₁-C₄)alkyl)amino, —NHCO₂R⁷, —N((C₁-C₄)alkyl)CO₂R⁷, —NHC(O)R⁷, or —N((C₁-C₄)alkyl)C(O)R⁷;
each R⁵ is independently selected from (C₁-C₆)alkyl, (C₁-C₆)haloalkyl, (C₃-C₆)cycloalkyl, halogen, cyano, hydroxyl, hydroxy(C₁-C₆)alkyl, (C₁-C₆)alkoxy, (C₁-C₄)alkoxy(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, and heterocycloalkyl;
R⁶ is hydrogen, (C₁-C₆)alkyl, (C₁-C₆)haloalkyl, (C₃-C₆)cycloalkyl, hydroxy(C₁-C₆)alkyl, (C₁-C₄)alkoxy(C₁-C₆)alkyl, —((C₀-C₃)alkyl)CO₂R⁷, —((C₀-C₃)alkyl)CONR⁷R⁸, aryl, heteroaryl, aryl(C₁-C₆)alkyl, heteroaryl(C₁-C₆)alkyl, or heterocycloalkyl;
R⁷ is hydrogen, (C₁-C₆)alkyl, (C₁-C₆)haloalkyl, (C₃-C₆)cycloalkyl, (C₁-C₄)alkoxy(C₁-C₆)alkyl, aryl, heteroaryl, aryl(C₁-C₆)alkyl, heteroaryl(C₁-C₆)alkyl, or heterocycloalkyl;
R⁸ is hydrogen, (C₁-C₆)alkyl, or (C₁-C₆)haloalkyl;
or R⁷ and R⁸ taken together with the nitrogen atom to which they are attached form a four to eight membered ring, optionally containing an additional heteroatom selected from oxygen, nitrogen, and sulfur, which ring is optionally substituted by (C₁-C₄)alkyl, (C₁-C₄)haloalkyl, (C₃-C₆)cycloalkyl, —CO₂H, —CO₂(C₁-C₄)alkyl, hydroxyl, hydroxy(C₁-C₆)alkyl, (C₁-C₄)alkoxy, (C₁-C₄)alkoxy(C₁-C₆)alkyl, amino, (C₁-C₄)alkylamino, or ((C₁-C₄)alkyl)((C₁-C₄)alkyl)amino;
R⁹ is —C(O)R⁷, —CO₂R⁷, —C(O)NR⁷R⁸, (C₁-C₆)alkyl, (C₁-C₆)haloalkyl, (C₃-C₆)cycloalkyl, aryl, heteroaryl, aryl(C₁-C₆)alkyl, heteroaryl(C₁-C₆)alkyl, or heterocycloalkyl, wherein said (C₁-C₆)alkyl, (C₁-C₆)haloalkyl, (C₃-C₆)cycloalkyl, aryl, heteroaryl, aryl(C₁-C₆)alkyl, heteroaryl(C₁-C₆)alkyl, or heterocycloalkyl is optionally substituted by —CO₂R⁷, —CONH₂, —CONH(C₁-C₄)alkyl, —CON((C₁-C₄)alkyl)((C₁-C₄)alkyl), hydroxyl, (C₁-C₄)alkoxy, amino, (C₁-C₄)alkylamino, ((C₁-C₄)alkyl)((C₁-C₄)alkyl)amino, —NHCO₂R⁷, —N((C₁-C₄)alkyl)CO₂R⁷, —NHC(O)R⁷, or —N((C₁-C₄)alkyl)C(O)R⁷;
or R⁸ and R⁹ taken together with the nitrogen atom to which they are attached form a four to eight membered ring, optionally containing an additional heteroatom selected from oxygen, nitrogen, and sulfur, which ring is optionally substituted by cyano, (C₁-C₄)alkyl, (C₁-C₄)haloalkyl, (C₃-C₆)cycloalkyl, —CO₂H, —CO₂(C₁-C₄)alkyl, —CONR⁷R⁸, hydroxyl, hydroxy(C₁-C₆)alkyl, (C₁-C₄)alkoxy, (C₁-C₄)alkoxy(C₁-C₆)alkyl, amino, (C₁-C₄)alkylamino, ((C₁-C₄)alkyl)((C₁-C₄)alkyl)amino, —NHCO₂R⁷, —N((C₁-C₄)alkyl)CO₂R⁷, —NHC(O)R⁷, or —N((C₁-C₄)alkyl)C(O)R⁷; and
R¹⁰ is (C₁-C₆)alkyl, (C₁-C₆)haloalkyl, (C₃-C₆)cycloalkyl, halogen, cyano, hydroxyl, hydroxy(C₁-C₆)alkyl, (C₁-C₆)alkoxy, (C₁-C₄)alkoxy(C₁-C₆)alkyl, —((C₀-C₃)alkyl)CO₂R⁷, —((C₀-C₃)alkyl)CONR⁷R⁸, amino(C₁-C₆)alkyl, ((C₁-C₄)alkyl)((C₁-C₄)alkyl)amino(C₁-C₆)alkyl, (C₁-C₄)alkylamino(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, or heterocycloalkyl;
provided that the compound is not (2-(4-((3,5-dimethylisoxazol-4-yl)methoxy)phenyl)-N-((2-methoxyphenyl)(1-methyl-1H-imidazol-2-yl)methyl)acetamide);
or a salt thereof.

2. The compound or salt according to claim 1, wherein m is 1 and n is 1 or 2.

3. The compound or salt according to claim 1, wherein X¹ and X⁵ are each independently selected from N, N⁺—O⁻, CH, and CR⁵, and X², X³, and X⁴ are each independently selected from CH and CR⁵, wherein at least one of X¹ and X⁵ is N or N⁺—O⁻ and 0-3 of X¹, X², X³, X⁴, and X⁵ are CR.

4. The compound or salt according to claim 1, wherein X¹ is a carbon atom substituted by halogen, (C₁-C₄)alkyl, (C₁-C₄)haloalkyl, cyano, (C₁-C₄)alkoxy, or ((C₁-C₄)alkyl)((C₁-C₄)alkyl)amino, and X², X³, X⁴, and X⁵ are each independently a carbon atom substituted by hydrogen, halogen, (C₁-C₄)alkyl, (C₁-C₄)haloalkyl, cyano, (C₁-C₄)alkoxy, or ((C₁-C₄)alkyl)((C₁-C₄)alkyl)amino, wherein 2-4 of X², X³, X⁴, and X⁵ are a carbon atom substituted by hydrogen.

5. The compound or salt according to claim 1, wherein Y¹ is NH or NCH₃ and Y² is a bond.

6. (canceled)

7. The compound or salt according to claim 1, wherein Cy is piperidinyl, piperazinyl, phenyl, pyridinyl, pyridazinyl, pyrazinyl, or pyrimidinyl, each of which is optionally substituted one or two times, independently, by (C₁-C₆)alkyl, (C₁-C₆)haloalkyl, halogen, cyano, (C₁-C₄)alkoxy, (C₁-C₄)alkyl)((C₁-C₄)alkyl)amino, —((C₀-C₃)alkyl)CO₂H, —((C₀-C₃)alkyl)CO₂(C₁-C₆)alkyl, or —((C₀-C₃)alkyl)CONH(C₁-C₆)alkyl.

8. The compound or salt according to claim 1, wherein Cy is phenyl, which is optionally substituted one or two times, independently, by halogen, (C₁-C₄)alkyl, (C₁-C₄)haloalkyl, cyano, (C₁-C₄)alkoxy, or ((C₁-C₄)alkyl)((C₁-C₄)alkyl)amino.

9. The compound or salt according to claim 1, wherein Z is a bond, O, or NH.

10. The compound or salt according to claim 1, wherein R¹ is (C₃-C₆)alkyl, (C₃-C₆)cycloalkyl, phenyl, furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, or triazinyl, wherein said phenyl, furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, or triazinyl is optionally substituted one or two times, independently, by halogen, (C₁-C₄)alkyl, (C₁-C₄)alkoxy, or ((C₁-C₄)alkyl)((C₁-C₄)alkyl)amino.

11. The compound or salt according to claim 1, wherein R¹ is phenyl or pyridinyl, each of which is optionally substituted one or two times, independently, by halogen, (C₁-C₄)alkyl, (C₁-C₄)alkoxy, or ((C₁-C₄)alkyl)((C₁-C₄)alkyl)amino.

12. The compound or salt according to claim 1, wherein R² is hydrogen or methyl.

13. The compound or salt according to claim 1, wherein R³ and R^{3a} are each independently hydrogen or methyl.

14. The compound or salt according to claim 1, wherein each R⁴ is independently selected from hydrogen, (C₁-C₄)alkyl, (C₁-C₄)haloalkyl, —OR⁹, and —NR⁸R⁹, wherein said (C₁-C₄)alkyl or (C₁-C₄)haloalkyl is optionally substituted by hydroxyl, —OR⁹, —CO₂R⁷, —CONR⁷R⁸, or —NR⁸R⁹.

15. The compound or salt according to claim 1, wherein each R⁴ is independently selected from hydrogen, (C₁-C₄)alkyl, (C₁-C₄)alkoxy, hydroxy(C₂-C₄)alkoxy, (C₁-C₄)alkoxy(C₂-C₄)alkoxy, amino(C₂-C₄)alkoxy, —O((C₁-C₃)alkyl)CO₂H, —O((C₁-C₃)alkyl)CO₂(C₁-C₄)alkyl, —O((C₁-C₃)alkyl)CONH₂, —O((C₁-C₃)alkyl)CONH(C₁-C₄)alkyl, and —O((C₁-C₃)alkyl)CON((C₁-C₄)alkyl)((C₁-C₄)alkyl).

16. The compound or salt according to claim 1, wherein each R^{4a} is independently selected from hydrogen and methyl.

17. (canceled)

18. The compound or salt according to claim 1, wherein A¹ and A⁴ are each independently selected from CH and C((C₁-C₄)alkyl), and one of A² and A³ is N((C₁-C₄)alkyl), O, or S and the other is N or CH.

19. A compound according to Formula (Ia): 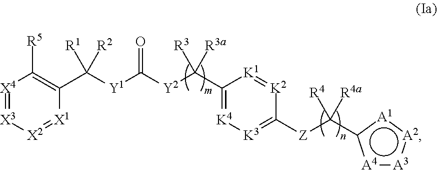
wherein:
m is 1;
n is 1 or 2;
X¹, X², X³, and X⁴ are each independently selected from N, N⁺—O⁻, CH, and CR⁵, wherein 0-2 of X¹, X², X³, and X⁴ are N or N⁺—O⁻ and 0-2 X¹, X², X³, and X⁴ are CR⁵;
Y¹ is NH or NCH₃ and Y² is a bond;
K¹, K², K³, and K⁴ are each independently selected from N, N⁺—O⁻, CH, and CR¹⁰, wherein 0-2 of K¹, K², K³, and K⁴ are N or N⁺—O⁻ and 0-2 of K¹, K², K³, and K⁴ are CR¹⁰;
Z is O, NR⁶, or a bond;
A¹, A², A³, and A⁴ are each independently selected from N, NR⁶, O, S, CH, and CR¹⁰, wherein one of A¹, A², A³, and A⁴ is NR⁶, O, or S, 0-2 of A¹, A², A³, and A⁴ are CR¹⁰, and 0-3 of A¹, A², A³, and A⁴ are CH or N;
R¹ is (C₃-C₆)alkyl, (C₃-C₆)haloalkyl, (C₃-C₈)cycloalkyl, (C₃-C₆)alkoxy, (C₁-C₆)alkoxy(C₁-C₂)alkyl, aryl, heteroaryl, aryl(C₁-C₆)alkyl, heteroaryl(C₁-C₆)alkyl, or heterocycloalkyl, each of which is optionally substituted one, two, or three times, independently, by R⁵;
R² is hydrogen, (C₁-C₆)alkyl, or (C₁-C₆)haloalkyl;
or R¹ and R² taken together with the carbon atom to which they are attached form a three to eight membered ring, optionally containing a heteroatom selected from oxygen, nitrogen, and sulfur, which ring is optionally substituted one, two, or three times, independently, by R⁵;
R³ and R^{3a} are each independently hydrogen, hydroxyl, (C₁-C₄)alkyl, (C₁-C₄)haloalkyl, halogen, (C₁-C₄)alkoxy, amino, (C₁-C₄)alkylamino, or ((C₁-C₄)alkyl)((C₁-C₄)alkyl)amino;
each R⁴ is independently selected from hydrogen, halogen, (C₁-C₄)alkyl, (C₁-C₄)haloalkyl, —OR⁹, and —NR⁸R⁹, wherein said (C₁-C₄)alkyl or (C₁-C₄)haloalkyl is optionally substituted by hydroxyl, —OR⁹, —CO₂R⁷, —CONR⁷R⁸, or —NR⁸R⁹;
each R^{4a} is independently selected from hydrogen, halogen, hydroxyl, amino, and (C₁-C₄)alkyl;
each R⁵ is independently selected from (C₁-C₆)alkyl, (C₁-C₆)haloalkyl, (C₃-C₆)cycloalkyl, halogen, cyano, hydroxyl, hydroxy(C₁-C₆)alkyl, (C₁-C₆)alkoxy, (C₁-C₄)alkoxy(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, and heterocycloalkyl;
R⁶ is hydrogen, (C₁-C₆)alkyl, (C₁-C₆)haloalkyl, (C₃-C₆)cycloalkyl, hydroxy(C₁-C₆)alkyl, (C₁-C₄)alkoxy(C₁-C₆)alkyl, —((C₀-C₃)alkyl)CO₂R⁷, —((C₀-C₃)alkyl)CONR⁷R⁸, aryl, heteroaryl, aryl(C₁-C₆)alkyl, heteroaryl(C₁-C₆)alkyl, or heterocycloalkyl;
R⁷ is hydrogen, (C₁-C₆)alkyl, (C₁-C₆)haloalkyl, (C₃-C₆)cycloalkyl, (C₁-C₄)alkoxy(C₁-C₆)alkyl, aryl, heteroaryl, aryl(C₁-C₆)alkyl, heteroaryl(C₁-C₆)alkyl, or heterocycloalkyl;
R⁸ is hydrogen, (C₁-C₆)alkyl, or (C₁-C₆)haloalkyl;
or R⁷ and R⁸ taken together with the nitrogen atom to which they are attached form a four to eight membered ring, optionally containing an additional heteroatom selected from oxygen, nitrogen, and sulfur, which ring is optionally substituted by (C₁-C₄)alkyl, (C₁-C₄)haloalkyl, (C₃-C₆)cycloalkyl, —CO₂H, —CO₂(C₁-C₄)alkyl, hydroxyl, hydroxy(C₁-C₆)alkyl, (C₁-C₄)alkoxy, (C₁-C₄)alkoxy(C₁-C₆)alkyl, amino, (C₁-C₄)alkylamino, or ((C₁-C₄)alkyl)((C₁-C₄)alkyl)amino;
R⁹ is —C(O)R⁷, —CO₂R⁷, —C(O)NR⁷R⁸, (C₁-C₆)alkyl, (C₁-C₆)haloalkyl, (C₃-C₆)cycloalkyl, aryl, heteroaryl, aryl(C₁-C₆)alkyl, heteroaryl(C₁-C₆)alkyl, or heterocycloalkyl, wherein said (C₁-C₆)alkyl, (C₁-C₆)haloalkyl, (C₃-C₆)cycloalkyl, aryl, heteroaryl, aryl(C₁-C₆)alkyl, heteroaryl(C₁-C₆)alkyl, or heterocycloalkyl is optionally substituted by —CO₂R⁷, —CONH₂, —CONH(C₁-C₄)alkyl, —CON((C₁-C₄)alkyl)((C₁-C₄)alkyl), hydroxyl, (C₁-C₄)alkoxy, amino, (C₁-C₄)alkylamino, ((C₁-C₄)alkyl)((C₁-C₄)alkyl)amino, —NHCO₂R⁷, —N((C₁-C₄)alkyl)CO₂R⁷, —NHC(O)R⁷, or —N((C₁-C₄)alkyl)C(O)R⁷;
or R⁸ and R⁹ taken together with the nitrogen atom to which they are attached form a four to eight membered ring, optionally containing an additional heteroatom selected from oxygen, nitrogen, and sulfur, which ring is optionally substituted by cyano, (C₁-C₄)alkyl, (C₁-C₄)haloalkyl, (C₃-C₆)cycloalkyl, —CO₂H, —CO₂(C₁-C₄)alkyl, —CONR⁷R⁸, hydroxyl, hydroxy(C₁-C₆)alkyl, (C₁-C₄)alkoxy, (C₁-C₄)alkoxy(C₁-C₆)alkyl, amino, (C₁-C₄)alkylamino, ((C₁-C₄)alkyl)((C₁-C₄)alkyl)amino, —NHCO₂R⁷, —N((C₁-C₄)alkyl)CO₂R⁷, —NHC(O)R⁷, or —N((C₁-C₄)alkyl)C(O)R⁷; and
R¹⁰ is (C₁-C₆)alkyl, (C₁-C₆)haloalkyl, (C₃-C₆)cycloalkyl, halogen, cyano, hydroxyl, hydroxy(C₁-C₆)alkyl, (C₁-C₆)alkoxy, (C₁-C₄)alkoxy(C₁-C₆)alkyl, —((C₀-C₃)alkyl)CO₂R⁷, —((C₀-C₃)alkyl)CONR⁷R⁸, amino(C₁-C₆)alkyl, ((C₁-C₄)alkyl)((C₁-C₄)alkyl)amino(C₁-C₆)alkyl, (C₁-C₄)alkylamino(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, or heterocycloalkyl;
or a pharmaceutically acceptable salt thereof.

20. The compound or salt according to claim 19, wherein:
X¹, X², X³, and X⁴ are each independently a carbon atom substituted by hydrogen, halogen, cyano, (C₁-C₄)alkyl, (C₁-C₄)haloalkyl, (C₁-C₄)alkoxy, or ((C₁-C₄)alkyl)((C₁-C₄)alkyl)amino, wherein 2-4 of X¹, X², X³, and X⁴ are a carbon atom substituted by hydrogen;
K¹, K², K³, and K⁴ are each independently a carbon atom substituted by hydrogen, halogen, (C₁-C₄)alkyl, (C₁-C₄)alkoxy, or ((C₁-C₄)alkyl)((C₁-C₄)alkyl)amino, wherein 2-4 of K¹, K², K³, and K⁴ are a carbon atom substituted by hydrogen;
Z is O, NH, —N(C₁-C₄)alkyl, —N((C₀-C₃)alkyl)CO₂R⁷, —N((C₀-C₃)alkyl)CONR⁷R⁸, or a bond;
A¹ and A⁴ are each independently selected from CH and CR¹⁰, and one of A² and A³ is NR⁶, O, or S and the other is N or CH;
R² is hydrogen;
R³ and R^{3a} are each independently hydrogen or methyl;
each R⁴ is independently selected from hydrogen, (C₁-C₄)alkyl, (C₁-C₄)alkoxy, hydroxy(C₂-C₄)alkoxy, (C₁-C₄)alkylamino, ((C₁-C₄)alkyl)((C₁-C₄)alkyl)amino, (C₁-C₄)alkoxy(C₁-C₄)alkylamino, (C₁-C₄)alkoxy(C₂-C₄)alkoxy, amino(C₂-C₄)alkoxy, —O((C₁-C₃)alkyl)CO₂H, —O((C₁-C₃)alkyl)CO₂(C₁-C₄)alkyl, —O((C₁-C₃)alkyl)CONH₂, —O((C₁-C₃)alkyl)CONH(C₁-C₄)alkyl, and —O((C₁-C₃)alkyl)CON((C₁-C₄)alkyl)((C₁-C₄)alkyl);
each R^{4a} is independently selected from hydrogen, hydroxyl, amino, and (C₁-C₄)alkyl;
R⁷ is hydrogen, (C₁-C₆)alkyl, (C₁-C₆)haloalkyl, (C₃-C₆)cycloalkyl, (C₁-C₄)alkoxy(C₁-C₆)alkyl, aryl, heteroaryl, aryl(C₁-C₆)alkyl, heteroaryl(C₁-C₆)alkyl, or heterocycloalkyl; and
R⁸ is hydrogen, (C₁-C₆)alkyl, or (C₁-C₆)haloalkyl.

21. The compound or salt according to claim 20, wherein:
Z is O, NH, —N(C₁-C₄)alkyl, or a bond;
A¹ and A⁴ are each independently selected from CH and C((C₁-C₄)alkyl), and one of A² and A³ is O or S and the other is N;
R¹ is phenyl optionally substituted one or two times, independently, by halogen, (C₁-C₄)alkyl, (C₁-C₄)haloalkyl, cyano, (C₁-C₄)alkoxy, or ((C₁-C₄)alkyl)((C₁-C₄)alkyl)amino; and
each R⁴ is independently selected from hydrogen, (C₁-C₄)alkyl, (C₁-C₄)alkylamino, ((C₁-C₄)alkyl)((C₁-C₄)alkyl)amino, and (C₁-C₄)alkoxy.

22. (canceled)

23. (canceled)

24. A pharmaceutical composition comprising the compound, or pharmaceutically acceptable salt thereof, according to claim 19 and a pharmaceutically acceptable excipient.

25-35. (canceled)


SEQUENCE LISTING (2 sequences)

SEQ ID NO: 1 - protein, 15 aa, Human
MKKHHHHHHLVPRGS

SEQ ID NO: 2 - protein, 25 aa, Artificial Sequence
  Biotinylated peptide
CPSSHSSLTERHKILHRLLQEGSPS